(19) United States
(12) Patent Application Publication
(10) Pub. No.: US 2014/0045717 A1
(43) Pub. Date: Feb. 13, 2014

(54) Single Nucleotide Polymorphism Biomarkers for Diagnosing Autism

(71) Applicant: Valerie Wailin Hu, Washington, DC (US)

(72) Inventor: Valerie Wailin Hu, Washington, DC (US)

(73) Assignee: THE GEORGE WASHINGTON UNIVERSITY, Washington, DC (US)

(21) Appl. No.: 13/993,017

(22) PCT Filed: Dec. 9, 2011
(86) PCT No.: PCT/US2011/064213

§ 371 Date: Oct. 30, 2013

Related U.S. Application Data
(60) Provisional application No. 61/422,066, filed on Dec. 10, 2010.

(51) Int. Cl. C12Q 1/68; G06F 19/22

(52) U.S. Cl. CPC C12Q 1/6883; G06F 19/22
USPC 506/9; 506/16; 702/19

(57) ABSTRACT

The invention provides methods of identifying biomarkers associated with autism or autism spectrum disorder based upon quantitative trait association analyses using genome-wide genotype data combined with case-control association analyses using distinct ASD phenotypes identified on the basis of symptomatic profiles, including deficits in language usage, non-verbal communication, social development, play skills, and insistence on sameness and rituals. Also provided are compositions identified using the methods of the invention and use thereof.

### FIELD OF THE INVENTION

This invention relates to compositions, methods and kits for aiding in the assessment and identification of autism spectrum disorders (“ASD”) in humans and methods for the identification of biomarkers for ASD.

### BACKGROUND OF THE INVENTION

Autism, or autism spectrum disorder (“ASD”), is a severe and relatively common neuropsychiatric disorder characterized by abnormalities in social behavior and communication skills, with tendencies towards patterns of abnormal repetitive movements and other behavior disturbances. Current prevalence estimates are 0.1-0.2% of the population for autism and 0.6% of the population for ASDs (Abrahams, B. S. & Geschwind, D. H. Advances in autism genetics: on the threshold of a new neurobiology. Nat Rev Genet 9, 341-55 (2008)). Globally, males are affected four times as often as females (Autism and Developmental Disabilities Monitoring Network. http://www.cdc.gov/mmwr/pdf/ss/ss5601.pdf. (2007)). As such, autism poses a major public health concern of unknown cause that extends into adulthood and places an immense economic burden on society. The most prominent features of autism are social and communication deficits. The former are manifested in reduced sociability (reduced tendency to seek or pay attention to social interactions), a lack of awareness of social rules, difficulties in social imitation and symbolic play, impairments in giving and seeking comfort and forming social relationships with other individuals, failure to use nonverbal communication such as eye contact, deficits in perception of others' mental and emotional states, lack of reciprocity, and failure to share experience with others. Communication deficits are manifested as a delay in or lack of language, impaired ability to initiate or sustain a conversation with others, and stereotyped or repetitive use of language. Autistic children have been shown to engage in free play much less frequently and at a much lower developmental level than peers of similar intellectual abilities. Markers of social deficits in affected children appear as early as 12-18 months of age, suggesting that autism is a neurodevelopmental disorder. It has been suggested that autism originates in developmental failure of neural systems governing social and emotional functioning. Although social and cognitive development are highly correlated in the general population, the degree of social impairment does not correlate well with IQ in individuals with autism. The opposite is seen in Down's syndrome and Williams syndrome, where social development is superior to cognitive function. Both examples point to a complex source of sociability. The etiology of the most common forms of autism is still unknown.

Hu et al. recently demonstrated differential gene expression in lymphoblastoid cell lines (LCL) from monozygotic twins discordant for diagnosis of autism (Hu, V. et al. (2006) BMC Genomics 7, 118), which strongly suggests that epigenetic factors are also involved in idiopathic autism. Other studies have suggested that “epigenetic hotspots” or regions susceptible to genomic imprinting are located in chromosomal regions (e.g., 15q and 7q) identified in genetic linkage analysis of autism (Schanen, N.C. (2006) Hum Mol Genet15 Spec No 2, R138-50; Davies, W. et al. (2001) Ann Med 33, 428-36). Hogart et al. (Hogart, A. et al. (2007) Hum Mol Genet16, 691-703) argues that genes located close to these hotspots (like genes encoding for GABAA-receptor subunits, GABRB3, GABRA5 and GABRG3), while not necessarily subject to imprinting, can still convey an ASD risk upon disrupted epigenetic regulation.

Autism spectrum disorders (ASD), including autistic disorder, pervasive developmental disorder-not otherwise specified (PDD-NOS), including atypical autism, Asperger's Disorder, thus represent a group of neurodevelopmental disorders that are characterized by impaired reciprocal social interactions, delayed or aberrant communication, and stereotyped, repetitive behaviors, often with restricted interests (American Psychological Association (1994) Diagnostic and Statistical Manual of Mental Disorders, (American Psychological Association, Washington, D.C.), Volkmar F R (1991) DSM-IV in progress. autism and the pervasive developmental disorders. Hosp Community Psychiatry 42: 33-5). With a concordance rate as high as 90% based on twin studies (Bailey A, et al (1995) Autism as a strongly genetic disorder: Evidence from a British twin study. Psychol Med 25: 63-77), ASD are among the most heritable of neuropsychiatric conditions. Yet, there are no unequivocal genetic markers for these disorders. Thus, a considerable amount of effort has been devoted to identifying genetic mutations or variants that associate with these perplexing and often devastating, life-long disorders.

In a recent paper, Hu et al demonstrated that the autistic population can be divided into at least 4 phenotypic subgroups on the basis of cluster analyses of 123 severity scores taken from each individual's diagnostic assessment using the Autism Diagnostic Interview-Revised (Hu V W & Steinberg M E (2009) Novel clustering of items from the Autism Diagnostic Interview-Revised to define phenotypes within autism spectrum disorders. Autism Res 2: 67-77). The resulting subgroups included one with severe language impairment, another with mild severity across all items, a third of intermediate severity, and a fourth with a higher frequency of savant skills. Hu et al further demonstrated by gene expression profiling of lymphoblastoid cell lines from 3 of these subgroups (excluding the intermediate) and nonautistic controls that cells from each of these subgroups exhibited differentially expressed genes relative to that of the controls, but also were distinguishable from each other in terms of unique, subtype-specific differentially expressed genes (Hu V W, et al. (2009) Gene expression profiling differentiates autism case-controls and phenotypic variants of autism spectrum disorders: Evidence for circadian rhythm dysfunction in severe autism. Autism Res 2: 78-97). These studies thus support the concept that different subgroups of autistic individuals may exhibit subtype-dependent biological differences due to genetic variation.

Because of the relatively high prevalence of ASD in the general population (˜1:110), genome-wide association (GWA) analyses have been used recently to search for common variants that may associate with increased susceptibility to this set of disorders (Wang K, et al (2009) Common genetic variants on 5p14.1 associate with autism spectrum disorders. Nature 459: 528-533; Ma D, et al (2009) A genome-wide association study of autism reveals a common novel risk locus at 5p14.1. Ann Hum Genet 73: 263-273; Weiss L A, et al (2009) A genome-wide linkage and association scan reveals novel loci for autism. Nature 461: 802-808; Anney R, et al (2010) A genome-wide scan for common alleles affecting risk for autism. Hum Mol Genet 19: 4072-4082.). However, despite case-control studies that have now exceeded many thousands of subjects and more than 500,000 single nucleotide polymorphisms, only a few significant single nucleotide polymorphisms have been identified. In addition, replication of these single nucleotide polymorphisms in independent studies has not been successful. The inability to replicate findings from GWA analyses may be in part due to the genetic heterogeneity of the autistic population, thus giving rise to increased “noise” in the data. This genetic heterogeneity is likely responsible for the well-noted phenotypic and symptomatic heterogeneity among individuals with autism.

Thus, there is a need for compositions and methods that will provide an increased understanding of the pathophysiology of autism spectrum disorders, such as autistic disorder, pervasive developmental disorders not otherwise specified (PDD-NOS), and Asperger's syndrome, and their treatment.

The present invention satisfied these and other needs by demonstrating herein that the combination of quantitative trait association analyses with subtype-dependent genetic association analyses of such ASD subtypes with single nucleotide polymorphisms that are identified and filtered according to their association with quantitative traits relevant to ASD reveal more significant single nucleotide polymorphisms with increased statistical power. The present invention thus provides ASD-specific single nucleotide polymorphisms compositions and methods for identifying such ASD-specific single nucleotide polymorphisms.

### SUMMARY OF THE INVENTION

In accordance with the present invention, methods and compositions are provided for diagnosis and treatment of autism spectrum disorders. “Autism” and “autism spectrum disorders” are used interchangeably herein.

In the present invention a genome wide association meta analysis is provided that demonstrates that in addition to multiple rare variations, part of the complex genetic architecture of autism involves certain common variations. Utilizing the compositions and methods disclosed herein certain biomarkers are identified as being associated with autism spectrum disorders and include certain single nucleotide polymorphisms (SNPs) which demonstrated statistically significant strong association with autism and/or autism risk in both the discovery and validation datasets. These findings further support this stepwise approach as depicted in FIG. 1 of first identifying quantitative trait loci relevant to characteristics of autism before applying case-control genetic association analyses to autism, in which the cases are divided into subtypes according to the methods of Hu and Steinberg (2009) to reduce the heterogeneity in the autistic population.

In one aspect of the present invention, a method is provided for identifying biomarkers for the diagnosis of autism spectrum disorders comprising (a) performing quantitative trait association analysis for at least one category of symptoms or related quantitative traits, to identify filtered set of single nucleotide polymorphisms that are associated with each quantitative trait; (b) performing case-control association analysis with each set of trait-associated single nucleotide polymorphisms in which cases are both combined and divided into from at least one to at least four ASD subtypes to identify trait associated single nucleotide polymorphisms that are subtype-dependent with a Bonferroni significance of P<0.05; (c) performing case control association analysis with the combined set of Bonferroni significant single nucleotide polymorphisms from analysis in step (b) to identify those novel ASD subtype-associated single nucleotide polymorphisms that are associated with each ASD subtype vs. controls and those novel ASD subtype-associated quantitative trait loci that are replicated in a second subtype.

In one embodiment of the method of the present invention, the method additionally comprises the additional step of (d) measuring the level of differential gene expression in one or more of biomarker-associated genes listed in Table 1 or Table 7.

In one embodiment of the method of the present invention, the method may be conducted in the absence of step c) and still yield one or more of the novel SNP biomarkers depicted in Table 7 infra.

In another embodiment of the present invention, quantitative severity criteria are assessed across at least one category of behavioral symptoms or quantitative traits of ASD subtypes comprising language deficits, deficits in nonverbal communication, under developed play skills, delayed social development, and insistence on sameness/ritualistic behaviors, separately or in combination with measuring the level of differential gene expression in one or more of the biomarker-associated genes listed in Table 1 or Table 7, or any combination thereof.

In yet another embodiment of the method of the present invention, the case-control association analysis of step (b) comprises a cluster analysis to divide the autistic cases into four phenotypic subgroups according to symptomatic severity profiles derived from the one to one hundred and twenty three items listed on the ADI-R assessments in Table 9 to reduce the behavioral/symptomatic and genetic heterogeneity among the cases within each subgroup.

In yet another embodiment of the cluster analysis of the case-control association analysis of step (b), the ADI-R assessments comprise items one to one hundred and twenty three (123), or any integer value therebetween of the published ADI-R assessments as described in Hu V W & Steinberg M E (2009) Novel clustering of items from the autism diagnostic interview-revised to define phenotypes within autism spectrum disorders. Autism Res 2: 67-77, incorporated by reference herein in its entirety.

In yet another embodiment of the method of the present invention, the four phenotypic subgroups obtained from the cluster analysis distinguish between different variants of autism spectrum disorder comprising a “mild” subgroup with lower severity scores across all ADIR items, a subgroup with intermediate severity across all ADIR items, a severely language-impaired subgroup with higher severity scores on spoken language items on the ADIR, a subgroup with a moderate severity profile, often with higher frequency of savant skills, or any combination thereof.

In yet another embodiment of the method of the present invention, the samples are assessed in a genome-wide association analysis (GWAS).

In yet another embodiment of the method of the present invention, the novel ASD subtype-associated single nucleotide polymorphisms that are associated with each quantitative trait and/or those novel ASD subtype-associated quantitative trait loci that are replicated in a second subtype ASD subtyping method either specifically exclude or specifically include those single nucleotide polymorphisms selected from the group consisting of rs4307059, rs7704909, rs12518194, rs4327572, rs1896731, and rs10038113, or variants, mutants, alleles or complementary sequences thereof, or any combination thereof.

In one embodiment of the method of the present invention, the autism spectrum disorder comprises autistic disorder, pervasive developmental disorder-not otherwise specified (PDD-NOS), including atypical autism, Asperger's Disorder, or a combination thereof.

In yet another embodiment of the diagnosing/screening method of the present invention, the healthy individual is a non-phenotypic discordant twin, sibling of the subject, or healthy, unrelated individual.

By using the aforementioned method for identifying biomarkers for the diagnosis of autism spectrum disorders, certain single nucleotide polymorphism biomarkers were identified.

Thus, in one aspect of the present invention, a biomarker is provided for the diagnosis of autism and autism spectrum disorders comprising at least one language impairment quantitative trait loci-specific single nucleotide polymorphism, at least one non-verbal communication quantitative trait loci-specific single nucleotide polymorphism, at least one play skills quantitative trait loci-specific single nucleotide polymorphism, at least one insistence on sameness/rituals quantitative trait loci-specific single nucleotide polymorphism, and/or at least one social skills and development quantitative trait loci-specific single nucleotide polymorphism, or variants, mutants, alleles or complementary sequences thereof, or any combination thereof.

In another aspect of the present invention, each of the biomarkers listed infra may further comprise an autism or autism spectrum disorder differentially expressed gene comprising one or more of the differentially expressed biomarker-associated genes listed in Table 1 or Table 7, or any combination thereof.

In one embodiment of the present invention, a biomarker is provided for the diagnosis of autism and autism spectrum disorders comprising at least one language impairment quantitative trait loci-specific single nucleotide polymorphism, at least one non-verbal communication quantitative trait loci-specific single nucleotide polymorphism, at least one play skills quantitative trait loci-specific single nucleotide polymorphism, at least one insistence on sameness/rituals quantitative trait loci-specific single nucleotide polymorphism, and/or at least one social skills and development quantitative trait loci-specific single nucleotide polymorphism wherein the aforementioned biomarkers comprise one or more of the biomarkers set forth in Table 1, variants, mutants, alleles or complementary sequences thereof, or any combination thereof. In one embodiment of the present invention, the biomarker may include one or more of those specific SNP biomarkers listed in Table 7 infra.

In one embodiment of the invention, a biomarker is provided for the diagnosis of autism and autism spectrum disorders comprising at least one language impairment quantitative trait loci-specific single nucleotide polymorphism set forth as: rs12407665, rs17828521, rs9474831, rs6454792, rs10183984, rs11969265, rs1231339, rs10806416, rs7785107, rs2277049, rs757099, rs7725785, rs758158, rs2287581, rs17830215, rs2180055, rs12893752, variants, mutants, alleles or complementary sequences thereof, or any combination thereof.

In one embodiment of the invention, a biomarker is provided for the diagnosis of autism and autism spectrum disorders comprising at least one non-verbal communication quantitative trait loci-specific single nucleotide polymorphism set forth as: rs9941626, rs13205238, rs11671930, rs11229410, rs11229413, rs11229411, rs11721070, rs12466917, rs13076171, rs7930778, rs12962411, rs12279895, rs730168, rs13021324, rs564127, rs1231339, rs393076, rs1938651, rs11138895, rs1938672, rs4804202, rs665036, rs4527692, rs519514, rs3133855, rs1938670, variants, mutants, alleles or complementary sequences thereof, or any combination thereof.

In one embodiment of the invention, a biomarker is provided for the diagnosis of autism and autism spectrum disorders comprising at least one play skills quantitative trait loci-specific single nucleotide polymorphism set forth as: rs13205238, rs1996893, rs12606567, rs3769845, rs2422675, rs4798405, rs10040891, rs8181738, rs11950809, rs11627027, rs1930, rs4894734, rs1482930, rs11671930, rs4980777, rs1481513, rs10987251, rs2151206, rs2044747, rs1440423, rs4745257, rs2779499, rs1796028, rs1888156, rs6734788, rs7605424, rs4627775, rs5009527, rs1796045, rs1863080, rs7337921, rs6452136, rs2168709, rs4386512, rs12614870, rs10491885, rs4646421, rs4894733, rs7944323, rs6791089, rs11229410, rs17770167, rs6698676, rs11664663, rs6482516, rs11082277, rs6988293, rs6974649, rs730168, rs1461710, rs9941626, rs3745651, rs9536962, rs7529505, rs9342127, rs1554547, rs9508456, rs2078520, rs9569991, rs3825597, rs3754741, rs2250595, rs1055518, rs2600685, variants, mutants, alleles or complementary sequences thereof, or any combination thereof.

In one embodiment of the invention, a biomarker is provided for the diagnosis of autism and autism spectrum disorders comprising at least one insistence on sameness or rituals quantitative trait loci-specific single nucleotide polymorphism set forth as: rs164187, rs3809854, rs3804967, rs3804968, rs317985, rs9634811, rs7819605, rs7950390, rs4436186, rs4838964, rs1827924, rs7699496, rs3861787, rs6782718, rs11038286, rs693442, rs1452885, rs17599556, rs185425, rs11035240, rs9693369, rs10781238, rs9568011, rs11682846, rs7650071, rs2574852, rs11914753, rs2469183, rs274646, rs13096022, rs17738966, rs6461176, variants, mutants, alleles or complementary sequences thereof, or any combination thereof.

In one embodiment of the invention, a biomarker is provided for the diagnosis of autism and autism spectrum disorders comprising at least one social skills and development quantitative trait loci-specific single nucleotide polymorphism set forth as: rs13205238, rs11138895, rs4809918, rs9479482, rs1294264, rs10788819, rs4959923, rs4905110, rs721087, rs12266938, rs10874468, rs13384439, rs4416176, rs10519124, rs12962411, rs6022029, rs11627027, rs6022039, rs10886048, rs4873815, rs4832481, rs3809282, rs1554547, rs2297172, rs2255313, rs2627468, rs12183587, rs10305860, rs30746, rs11138885, rs1294293, rs12115722, rs6698676, rs10997162, rs4646421, rs4778640, rs10110252, rs1996893, rs12811136, rs17192980, rs4811895, rs2519866, rs2779499, or rs2151206, variants, mutants, alleles or complementary sequences thereof, or any combination thereof.

In one aspect of the invention, a biomarker is provided for the diagnosis of autism and autism spectrum disorders comprising at least one combined quantitative trait loci-specific and ASD sub-type language impaired-specific single nucleotide polymorphism, at least one combined quantitative trait loci-specific and ASD sub-type intermediate-specific single nucleotide polymorphism, at least one combined quantitative trait loci-specific and ASD sub-type moderate-specific single nucleotide polymorphism, or at least one combined quantitative trait loci-specific and ASD sub-type mild-specific single nucleotide polymorphism, or variants, mutants, alleles or complementary sequences thereof, or any combination thereof.

In one aspect of the invention, a biomarker is provided for the diagnosis of autism and autism spectrum disorders comprising at least one combined quantitative trait loci-specific and ASD sub-type specific single nucleotide polymorphism set forth as rs2277049, rs757099, rs7785107, rs7725785, rs2287581, rs1231339, rs2180055, rs11671930, rs7950390, rs12266938, rs3861787, rs1827924, rs17738966, rs317985, rs730168, rs10519124, rs6482516, or rs2297172, variants, mutants, alleles or complementary sequences thereof, or any combination thereof.

In one aspect of the invention, a biomarker is provided for the diagnosis of autism and autism spectrum disorders comprising at least one combined quantitative trait loci-specific and ASD sub-type language impaired-specific single nucleotide polymorphism set forth as: rs2277049, rs757099, rs7785107, rs7725785, rs2287581, rs1231339, rs2180055, or rs11671930, variants, mutants, alleles or complementary sequences thereof, or any combination thereof.

In one aspect of the invention, a biomarker is provided for the diagnosis of autism and autism spectrum disorders comprising at least one combined quantitative trait loci-specific and ASD sub-type intermediate-specific single nucleotide polymorphism set forth as: rs7785107, rs7950390, rs12266938, or rs3861787, variants, mutants, alleles or complementary sequences thereof, or any combination thereof.

In one aspect of the invention, a biomarker is provided for the diagnosis of autism and autism spectrum disorders comprising at least one combined quantitative trait loci-specific and ASD sub-type moderate-specific single nucleotide polymorphism set forth as: rs1827924, rs17738966, rs7950390, rs3861787, or rs317985, variants, mutants, alleles or complementary sequences thereof, or any combination thereof.

In one aspect of the invention, a biomarker is provided for the diagnosis of autism and autism spectrum disorders comprising at least one combined quantitative trait loci-specific and ASD sub-type mild-specific single nucleotide polymorphism set forth as: rs12266938, rs730168, rs10519124, rs6482516, rs11671930, rs2297172, rs317985, rs1827924, rs1231339, rs757099, or rs7725785, variants, mutants, alleles or complementary sequences thereof, or any combination thereof.

In one aspect of the invention, a biomarker associated with more than one ASD subtype is provided for the diagnosis of autism and autism spectrum disorders comprising at least one combined quantitative trait loci-specific and ASD sub-type language impaired and ASD sub-type moderate and ASD subtype mild-specific single nucleotide polymorphism set forth as: rs317985, rs7785107, rs11671930, rs7950390, rs12266938, rs3861787, rs7725785, rs1827924, rs1231339, and rs757099, variants, mutants, alleles or complementary sequences thereof, or any combination thereof.

In yet another embodiment of the present invention, a biomarker is provided for the diagnosis of autism and autism spectrum disorders comprising at least one combined quantitative trait loci-specific and ASD sub-type language impaired-specific single nucleotide polymorphism set forth as: rs2277049, rs7725785, rs2287581, or rs11671930; at least one combined quantitative trait loci-specific and ASD sub-type intermediate-specific single nucleotide polymorphism set forth as: rs7950390; at least one combined quantitative trait loci-specific and ASD sub-type moderate-specific single nucleotide polymorphism set forth as: rs1827924, rs17738966, rs7950390, rs77255785, at least one combined quantitative trait loci-specific and ASD sub-type mild-specific single nucleotide polymorphism set forth as: rs730168, rs6482516, rs11671930, rs2297172, rs1827924, rs1231339, rs757099, rs7725785, variants, mutants, alleles or complementary sequences thereof, or any combination thereof, wherein the single nucleotide polymorphism is either directly associated with or indirectly associated with a gene selected from the group consisting of HTR4, GCH1, LDHD, CCL25, CCL20, TRIM65, NSUN6, PTAR1, and CDH6, each of which are a significantly differentially expressed gene by large-scale gene expression profiling of subtypes of ASD (FDR<5%).

In yet another embodiment of the present invention, a biomarker is provided for the diagnosis of autism and autism spectrum disorders comprising at least one combined quantitative trait loci-specific and ASD sub-type specific single nucleotide polymorphism set forth as: rs757099, rs77851107, rs1231339, rs2180055, rs12266938, rs3861787, rs317985, or rs317985, variants, mutants, alleles or complementary sequences thereof, or any combination thereof, wherein the single nucleotide polymorphism resides within intergenic regions that can be associated by band position to rare copy number variants (CNV) identified for ASD.

In one embodiment of the aforementioned biomarkers, the autism spectrum associated disorder comprises autistic disorder, pervasive developmental disorder-not otherwise specified (PDD-NOS), including atypical autism, Asperger's Disorder, or a combination thereof.

In one embodiment of the present invention, a microarray is provided having a plurality of different oligonucleotides with specificity for at least one single nucleotide polymorphism set forth in Table 1 or Table 7, or variants, mutants, alleles or complementary sequences thereof, or a combination thereof which are associated with at least one autism spectrum disorder, wherein the autism spectrum disorder comprises autistic disorder, pervasive developmental disorder-not otherwise specified (PDD-NOS), including atypical autism, Asperger's Disorder, or a combination thereof.

In another embodiment of the present invention, a microarray having a plurality of different oligonucleotides with specificity for at least one single nucleotide polymorphism set forth as rs2277049, rs757099, rs7785107, rs7725785, rs2287581, rs1231339, rs2180055, rs11671930, rs7950390, rs12266938, rs3861787, rs1827924, rs17738966, rs317985, rs730168, rs10519124, rs6482516, or rs2297172, or variants, mutants, alleles or complementary sequences thereof, or any combination thereof, which are associated with at least one autism spectrum disorder, wherein the autism spectrum disorder comprises autistic disorder, pervasive developmental disorder-not otherwise specified (PDD-NOS), including atypical autism, Asperger's Disorder, or a combination thereof.

In one aspect of this invention are microarrays comprising oligonucleotides specific for the SNPs described herein for use in a method for aiding in the diagnosis of or detecting a propensity for developing autism or an autism spectrum disorder in a patient in need thereof comprising detecting the presence of at least one SNP in the DNA of a patient suspected of having a propensity or increased risk for developing an autism spectrum disorder wherein the SNP comprises one or more of the SNPs in Tables 1 or 7 and wherein if at least one SNP is in the patient, the patient has a propensity or an increased risk for developing the autism spectrum disorder. The plurality of different oligonucleotides may be specific for SNPs comprising, e.g., (a) rs2277049, rs757099, rs7785107, rs7725785, rs2287581, rs1231339, rs2180055, rs11671930, rs7950390, rs12266938, rs3861787, rs1827924, rs17738966, rs317985, rs730168, rs10519124, rs6482516, or rs2297172, or (b) rs2277049, rs757099, rs7785107, rs7725785, rs2287581, rs1231339, rs2180055, rs11671930, rs7950390, rs12266938, rs3861787, rs1827924, rs17738966, rs317985, rs730168, rs10519124, rs6482516, and rs2297172 or (c) rs2277049, rs757099, rs7785107, rs7725785, rs2287581, rs1231339, rs2180055, and rs11671930, which are all associated with the language impairment subtype; or (d) rs7785107, rs7950390, rs12266938, and rs3861787, which are all associated with the intermediate subtype; rs1827924, rs17738966, rs11671930, rs3861787, or (e) rs317985 which are all associated with the moderate subtype, and rs12266938, rs730168, rs10519124, rs6482516, rs11671930, rs2297172, rs317985, rs1827924, rs1231339, rs757099, and rs7725785, which are all associated with the mild subtype as set forth in Table 7. The compositions may comprise, or be, microarrays comprising the plurality of different oligonucleotides with specificity for the SNPs.

In another aspect of the present invention a method is provided for diagnosing a patient with an autism spectrum disorder comprising identifying in a patient a biomarker or biomarker set comprising at least one single nucleotide polymorphism set forth as rs2277049, rs757099, rs7785107, rs7725785, rs2287581, rs1231339, rs2180055, rs11671930, rs7950390, rs12266938, rs3861787, rs1827924, rs17738966, rs317985, rs730168, rs10519124, rs6482516, or rs2297172, or variants, mutants, alleles or complementary sequences thereof, or any combination thereof; and, diagnosing a patient with autism or autism spectrum disorders.

In one embodiment of the present invention, a method is provided for diagnosing a patient pre-natally or post-natally with an autism spectrum disorder comprising detecting at least one single nucleotide polymorphism set forth as rs2277049, rs757099, rs7785107, rs7725785, rs2287581, rs1231339, rs2180055, rs11671930, rs7950390, rs12266938, rs3861787, rs1827924, rs17738966, rs317985, rs730168, rs10519124, rs6482516, or rs2297172, or variants, mutants, alleles or complementary sequences thereof, or any combination thereof; and, diagnosing a patient with an autism spectrum disorder.

In various embodiments of the diagnosing method of the present invention, the autism spectrum disorder comprises autistic disorder, pervasive developmental disorder-not otherwise specified (PDD-NOS), including atypical autism, Asperger's Disorder, or a combination thereof.

In another aspect of the invention, a method is provided for detecting a propensity for developing autism or autistic spectrum disorder in a patient in need thereof.

In yet another embodiment of the invention, a screening method is provided for detecting in a patient in need thereof a propensity or increased risk for developing autism or autistic spectrum disorder that entails detecting the presence of at least one single nucleotide polymorphism in a target polynucleotide wherein if said at least one single nucleotide polymorphism is present, said patient has an increased risk for developing autism and/or autistic spectrum disorder, wherein said single nucleotide polymorphism comprises or is selected from the group consisting of single nucleotide polymorphism set forth as rs2277049, rs757099, rs7785107, rs7725785, rs2287581, rs1231339, rs2180055, rs11671930, rs7950390, rs12266938, rs3861787, rs1827924, rs17738966, rs317985, rs730168, rs10519124, rs6482516, or rs2297172, or variants, mutants, alleles or complementary sequences thereof, or any combination thereof. In an embodiment of the invention the likelihood of a subject having a propensity or risk for developing an autism spectrum disorder increases as the number of SNPs from Tables 1 or 7 present in the subject increases.

In one embodiment of the screening method, the autism spectrum disorder comprises autistic disorder, pervasive developmental disorder-not otherwise specified (PDD-NOS), including atypical autism, Asperger's Disorder, or a combination thereof.

In one embodiment, the invention also provides at least one isolated autism-related SNP-containing nucleic acid identified using the aforementioned screening method wherein the autism-related SNP-containing nucleic acid is selected from the group consisting of rs2277049, rs757099, rs7785107, rs7725785, rs2287581, rs1231339, rs2180055, rs11671930, rs7950390, rs12266938, rs3861787, rs1827924, rs17738966, rs317985, rs730168, rs10519124, rs6482516, or rs2297172, or variants, mutants, alleles or complementary sequences thereof, or any combination thereof.

In another aspect, the present invention also provides for expression of SNP-containing nucleic acids exemplified in Table 2, or variants, mutants, alleles or complementary sequences thereof, or any combination thereof that may optionally be contained in a suitable expression vector.

In one aspect of the present invention, a method is provided for identifying a biomarker for the diagnosis of autism and autism spectrum disorders comprising obtaining a sample from individuals and their families and purifying genomic DNA from the sample; genotyping single nucleotide polymorphisms (SNP); assessing the single nucleotide polymorphisms; and, identifying a biomarker for the diagnosis of autism and autism spectrum disorders.

In yet another aspect of the present invention, an in vitro diagnostic test is provided for diagnosing or predicting autism spectrum disorders in an individual, the in vitro diagnostic test comprising at least one laboratory test for assaying a genetic sample from the individual for the presence of at least one allele of a biomarker associated with autism spectrum disorders; wherein the presence in the genetic sample of the at least one allele of a biomarker associated with autism spectrum disorders indicates that the individual is affected with autism spectrum disorders or predisposed to autism spectrum disorders.

In one embodiment of the in vitro diagnostic test of the present invention, the at least one allele of the biomarker associated with autism spectrum disorders is a single nucleotide polymorphism comprising rs2277049, rs757099, rs7785107, rs7725785, rs2287581, rs1231339, rs2180055, rs11671930, rs7950390, rs12266938, rs3861787, rs1827924, rs17738966, rs317985, rs730168, rs10519124, rs6482516, or rs2297172, mutants, alleles or complementary sequences thereof, or any combination thereof.

In one embodiment of the in vitro diagnostic test of the present invention, the at least one laboratory test for assaying the presence of at least one allele of a biomarker associated with autism spectrum disorders comprises an array based assay such as a microarray.

In yet another aspect of the invention, a method is provided for diagnosing a patient with autism or autism spectrum disorder comprising identifying in a patient a biomarker or biomarker set comprising (a) preparing samples of control and experimental DNA, wherein the experimental DNA is generated from a nucleic acid sample isolated from a subject suspected of being afflicted with the at least one autism spectrum disorder and the control DNA is generated from a nucleic acid sample isolated from a healthy individual; (b) preparing one or more microarrays comprising a plurality of different oligonucleotides having specificity for at least one allele of the biomarker associated with autism spectrum disorders comprising a single nucleotide polymorphism set forth as rs2277049, rs757099, rs7785107, rs7725785, rs2287581, rs1231339, rs2180055, rs11671930, rs7950390, rs12266938, rs3861787, rs1827924, rs17738966, rs317985, rs730168, rs10519124, rs6482516, or rs2297172, or variants, mutants, alleles or complementary sequences thereof, or any combination thereof. associated with the at least one autism spectrum disorder; (c) applying the prepared samples to the one or more microarrays to allow hybridization between the oligonucleotides and the control DNA and the oligonucleotide and the experimental DNA; (d) identifying the oligonucleotides on the microarray which display differential hybridization to the experimental DNA relative to the control DNA thereby identifying in a patient a biomarker or biomarker set profile for the at least one autism spectrum disorder.

In various embodiments of the diagnosing methods of the present invention, the autism spectrum disorder comprises autistic disorder, pervasive developmental disorder-not otherwise specified (PDD-NOS), including atypical autism, Asperger's Disorder, or a combination thereof.

In other aspects of the present invention, the biomarkers are useful for the identification of new agents, drugs or for testing the efficacy of compounds in the treatment of autism and autism spectrum disorders.

In one embodiment of the present invention, a method of identifying a candidate agent for treating autism or autism spectrum disorders is provided said method comprising: (a) contacting a biological sample from a patient with the candidate agent and determining the level of gene expression of one or more of the genes in Tables 1, or 7, associated with one or more of the biomarkers described herein; (b) determining the level of expression of a corresponding the level of gene expression of one or more of the genes in a biological sample not contacted with the candidate agent; (c) observing the effect of the candidate agent by comparing the level of expression of the genes in the biological sample contacted with the candidate agent and the level of expression of the corresponding genes in the biological sample not contacted with the candidate agent; and (d) identifying the agent from the observed effect, wherein an at least 1%, 2%, 5%, 10% difference between the level of expression of the gene or combination of genes in the biological sample contacted with the candidate agent and the level of expression of the corresponding gene or combination of genes in the biological sample not contacted with the candidate agent is an indication of an effect of the candidate agent.

In one embodiment of the candidate agent identifying method, the biomarker is a biomarker for diagnostically distinguishing between autism and autism spectrum disorders comprising at least one single nucleotide polymorphism set forth as: rs2277049, rs757099, rs7785107, rs7725785, rs2287581, rs1231339, rs2180055, rs11671930, rs7950390, rs12266938, rs3861787, rs1827924, rs17738966, rs317985, rs730168, rs10519124, rs6482516, or rs2297172, or variants, mutants, alleles or complementary sequences thereof, or any combination thereof.

In another embodiment of the invention, a pharmaceutical preparation comprising an agent according to the invention is provided.

In another embodiment of the invention, a method of producing a drug comprising the steps of the candidate agent identifying method according to the invention (i) synthesizing the candidate agent identified in step (c) above or an analog or derivative thereof in an amount sufficient to provide said drug in a therapeutically effective amount to a subject; and/or (ii) combining the drug candidate the candidate agent identified in step (c) above or an analog or derivative thereof with a pharmaceutically acceptable carrier.

In one embodiment of the present invention a method is provided for identifying agents which alter those neurological functions and disorders associated with autism pathophysiology comprising (a) providing cells expressing at least one allele of the biomarker associated with autism spectrum disorders comprising a single nucleotide polymorphism set forth as rs2277049, rs757099, rs7785107, rs7725785, rs2287581, rs1231339, rs2180055, rs11671930, rs7950390, rs12266938, rs3861787, rs1827924, rs17738966, rs317985, rs730168, rs10519124, rs6482516, or rs2297172, or variants, mutants, alleles or complementary sequences thereof, or any combination thereof associated with the at least one autism spectrum disorder; (b) providing cells which express the cognate wild type sequences corresponding to the single nucleotide polymorphism-containing nucleic acids; (c) contacting the cells from each sample with a test agent and analyzing whether said agent alters the neurological functions and disorders associated with autism pathophysiology of step a) relative to those of step b), thereby identifying agents which alter neurological functions and disorders associated with autism pathophysiology.

In yet another embodiment of the present invention, the aforementioned method is used to identify those agents that alter those neurological functions and disorders associated with autism pathophysiology comprising neuronal signaling and/or morphology, cell growth and death, embryogenesis, chromatin remodeling, myelination, oligodendrocyte differentiation, and complement activation, in addition to disorders that include demyelinating diseases, neuron dysfunction, nerve degeneration, and inflammation or cadherin-mediated cellular adhesion, or any combination thereof.

In yet another embodiment of the present invention, the aforementioned method is used to identify those agents that alter nervous system development, axon guidance, synaptic transmission or plasticity, long-term potentiation, neuron toxicity, Purkinje cell differentiation, cerebella development, embryonic development, regulation of actin networks, digestion, inflammation, oxidative stress, epilepsy, apoptosis, morphogenesis, cell survival, differentiation, the unfolded protein response, Type II diabetes and insulin signaling, digestion, liver toxicity (hepatic stellate cell activation, fibrosis, and cholestasis), endocrine function, circadian rhythm, cholesterol metabolism and the steroidogenesis pathway, or any combination thereof.

In yet another aspect, the present invention also provides a method of identifying an effective treatment regimen for a subject with an autism spectrum disorder, comprising detecting one or more biomarkers described in embodiments of the invention and correlating with an effective treatment regimen for an autism spectrum disorder.

In another embodiment, the present invention provides a method of identifying an effective treatment regimen for a subject with an autism spectrum disorder, comprising: a) correlating the presence of one or more biomarkers in a test subject with an autism spectrum disorder for whom an effective treatment regimen has been identified; and b) detecting the one or more markers of step (a) in the subject, thereby identifying an effective treatment regimen for the subject. Subjects who respond well to particular treatment protocols can thus be analyzed for specific biomarkers and a correlation can be established according to the methods provided herein. Alternatively, subjects who respond poorly to a particular treatment regimen can also be analyzed for particular biomarkers correlated with the poor response. Then, a subject who is a candidate for treatment for an autism spectrum disorder can be assessed for the presence of the appropriate biomarkers and the most appropriate treatment regimen can be provided.

In yet another embodiment of the effective treatment regimen method of the present invention, the subject undergoes a selected physiological change as a result of treatment, wherein the selected physiological change includes one or more improvements in social interaction, language abilities, restricted interests, repetitive behaviors, sleep disorders, seizures, gastrointestinal, hepatic, and mitochondrial function, neural inflammation, or a combination thereof.

In various embodiments of the effective treatment regimen method of the present invention, the autism spectrum disorder (ASD) comprises autistic disorder, pervasive developmental disorder-not otherwise specified (PDD-NOS), including atypical autism, Asperger's Disorder, or a combination thereof.

In yet another embodiment of the present invention, a method is provided for predicting efficacy of a test compound for altering a behavioral response in a subject with at least one autism spectrum disorder comprising: (a) preparing a microarray comprising a plurality of different oligonucleotides, wherein the oligonucleotides have specificity for at least one allele of the biomarker associated with ASD comprising a single nucleotide polymorphism set forth as rs2277049, rs757099, rs7785107, rs7725785, rs2287581, rs1231339, rs2180055, rs11671930, rs7950390, rs12266938, rs3861787, rs1827924, rs17738966, rs317985, rs730168, rs10519124, rs6482516, or rs2297172, or variants, mutants, alleles or complementary sequences thereof, or any combination thereof associated with at least one autism spectrum disorder; (b) obtaining a differential biomarker profile representative of the biomarker profile of at least one sample of a selected tissue type from a subject subjected to each of at least one of a plurality of selected behavioral therapies which promote the behavioral response; (c) administering the test compound to the subject; and (d) comparing a differential biomarker profile data in at least one sample of the selected tissue type from the subject treated with the test compound to determine a degree of similarity with one or more differential biomarker profile associated with an autism spectrum disorder; wherein the predicted efficacy of the test compound for altering the behavioral response is correlated to said degree of similarity.

In yet another embodiment of the compound efficacy testing method of the present invention, step (a) comprises obtaining a differential biomarker profile representative of the differential biomarker profile of at least two samples of a selected tissue type.

In yet another embodiment of the compound efficacy testing method of the present invention, the selected tissue type comprises a neuronal tissue type.

In yet another embodiment of the compound efficacy testing method of the present invention, the neuronal tissue type is selected from the group consisting of olfactory bulb cells, cerebrospinal fluid, hypothalamus, amygdala, pituitary, nervous system, brainstem, cerebellum, cortex, frontal cortex, hippocampus, striatum, and thalamus.

In yet another embodiment of the compound efficacy testing method of the present invention, the selected tissue type is selected from the group consisting of lymphocytes, blood, mucosal epithelial cells, brain, spinal cord, heart, arteries, esophagus, stomach, small intestine, large intestine, liver, pancreas, lungs, kidney, urinary tract, ovaries, breasts, uterus, testis, penis, colon, prostate, bone, muscle, cartilage, thyroid gland, adrenal gland, pituitary, bone marrow, blood, thymus, spleen, lymph nodes, skin, eye, ear, nose, teeth or tongue.

In yet another embodiment of the compound efficacy testing method of the present invention, the test compound is an antibody, a nucleic acid molecule, a small molecule drug, or a nutritional or herbal supplement.

In yet another embodiment of the compound efficacy testing method of the present invention, the behavioral therapy comprises applied behavior analysis (ABA) intervention methods, dietary changes, exercise, massage therapy, group therapy, talk therapy, play therapy, conditioning, or alternative therapies such as sensory integration and auditory integration therapies.

In various embodiments of the compound efficacy predicting method of the present invention, the autism spectrum disorder comprises autistic disorder, pervasive developmental disorder-not otherwise specified (PDD-NOS), including atypical autism, Asperger's Disorder, or a combination thereof.

In one embodiment of the methods for determining a biomarker profile for the administration of a therapeutic treatment, administration of therapeutic treatment results in a physiological change in the subject, such as a beneficial change. In a specific embodiment, the physiological change comprises one or more improvements in social interaction, language abilities, restricted interests, repetitive behaviors, sleep disorders, seizures, gastrointestinal, hepatic, and mitochondrial function, neural inflammation, or a combination thereof. In another embodiment, control DNA may be derived from the subject(s) prior to administration of the therapeutic treatment, or from a subject or group of subjects who do not receive the therapeutic treatment.

In yet another embodiment of the method of the present invention, prior to administration of behavioral therapy, the subject shows at least one symptom of a psychological or physiological abnormality.

In yet another embodiment of the method of the present invention, the neuronal tissue type is selected from the group consisting of olfactory bulb cells, cerebrospinal fluid, hypothalamus, amygdala, pituitary, nervous system, brainstem, cerebellum, cortex, frontal cortex, hippocampus, striatum, and thalamus.

In one embodiment of each of the aforementioned methods of the present invention, the use of the biomarkers of Table 1 or Table 7 specifically excludes those single nucleotide polymorphisms biomarkers associated with a cadherin gene [(cadherin gene 10 (CDH1O) and cadherin gene 9 (CDH9)] and/or protocadherin gene.

In yet another embodiment of each of the aforementioned methods of the present invention, the novel ASD subtype-associated single nucleotide polymorphisms that are associated with each quantitative trait and/or those novel ASD subtype-associated quantitative trait loci that are replicated in a second subtype ASD subtyping method either specifically exclude or specifically include those single nucleotide polymorphisms selected from the group consisting of rs4307059, rs7704909, rs12518194, rs4327572, rs1896731, and rs10038113, or variants, mutants, alleles or complementary sequences thereof, or any combination thereof.

In yet another aspect of the invention, kits are provided for use in the autism and autism spectrum disorder diagnosing, screening or candidate agent identifying methods described above comprising one or more of the autism and autism spectrum disorders single nucleotide polymorphism biomarkers or biomarker set profiles set forth in either Table 1, Table 2, Table 3, Table 4, Table 5, Table 6, Table 7, or Table 8, variants, mutants, alleles or complementary sequences thereof, or any combination thereof associated with at least one autism spectrum disorder.

In yet another aspect of the invention, a computer-readable medium on which is encoded programming code for analyzing and/or distinguishing between autism spectrum disorders from a plurality of data points wherein the computer-readable medium comprises a biomarker or biomarker profile set for diagnosing autism and autism spectrum disorders comprising at least one single nucleotide polymorphism set forth as: rs2277049, rs757099, rs7785107, rs7725785, rs2287581, rs1231339, rs2180055, rs11671930, rs7950390, rs12266938, rs3861787, rs1827924, rs17738966, rs317985, rs730168, rs10519124, rs6482516, or rs2297172, or variants, mutants, alleles or complementary sequences thereof, or any combination thereof.

In some embodiments, the methods of correlating biomarkers with diagnosing and/or treatment regimens can be carried out using a computer database.

Thus in one embodiment, the present invention provides a computer-assisted method of identifying a proposed treatment for autistic disorder comprising the steps of (a) storing a database of biological data for a plurality of patients, the biological data that is being stored including for each of said plurality of patients (i) a treatment type, (ii) at least one biomarker associated with an autism spectrum disorder wherein the at least one biomarker comprises rs2277049, rs757099, rs7785107, rs7725785, rs2287581, rs1231339, rs2180055, rs11671930, rs7950390, rs12266938, rs3861787, rs1827924, rs17738966, rs317985, rs730168, rs10519124, rs6482516, or rs2297172, or variants, mutants, alleles or complementary sequences thereof, or any combination thereof and (iii) at least one disease progression measure for an autism spectrum disorder from which treatment efficacy can be determined; and then (b) querying the database to determine the dependence on said biomarker of the effectiveness of a treatment type in treating autism spectrum disorder, to thereby identify a proposed treatment as an effective treatment for a subject carrying a biomarker correlated with autism spectrum disorder.

In yet another embodiment of the invention, in each of the screening methods, SNP biomarker profiling methods, drug discovery methods, compound efficacy testing methods, computer program for determining a biomarker profile, and kits specifically provided for supra (and infra) may also be, without any limitation, made and/or practiced with from at least one to at least 167, or any integer value thereof, different single nucleotide polymorphism biomarkers set forth in either Table 1, Table 2, Table 3, Table 4, Table 5, Table 6, Table 7, Table 8, or variants, mutants, alleles or complementary sequences thereof, or any combination thereof.

### BRIEF DESCRIPTION OF THE DRAWINGS

The foregoing and other aspects and advantages of the invention will be appreciated more fully from the following further description thereof, with reference to the accompanying drawings wherein:

FIG. 1 depicts a diagram of study design illustrating sequential application of quantitative trait and subphenotype association analyses. The quantitative trait association analyses were performed using the complete set of SNPs to prioritize SNPs that may have functional relevance to traits associated with ASD. This had the net effect of filtering the set of SNPs from over 500,000 to 167 QTL. In the second phase, each set of trait-associated SNPs (QTL) were employed in association analyses with the combined cases as well as with each ASD subtype. From these analyses, only 18 SNPs with Bonferroni-adjusted p-values <0.05 across all 5 traits and subtypes were combined for the final set of genetic association analyses using combined cases as well as ASD subtypes against controls.

FIG. 2A) depicts a Venn diagram showing unique and shared SNPs across ASD subtypes. FIG. 2B) depicts a table listing the shared SNPs and odds ratios in different ASD subtypes. Shading indicates SNPs with p-values that are <0.09 according to FDR_BH, while the unshaded SNPs have Bonferroni-adjusted p-values <0.05. For both (A) and (B), the subtypes are color-coded as follows: Red—Language-impaired; Green—Intermediate; Yellow—Moderate; Blue—Mild.

FIG. 3 depicts a gene interaction network of the genes (highlighted in blue) associated with the intronic SNPs identified in Table 7. Genes in the network are shown in pink while small molecules are green. Processes are shown in yellow and disorders are shown in purple. The orange entities represent functional complexes.

FIG. 4 depicts Quantitative trait profiles generated by summing the severity scores for ADI-R items for each trait listed in Table 9. The Y axis is the cumulative ADI-R severity score for particular trait. The X axis represents the population of individuals from the lowest (left) to highest severity scores (right).

FIG. 5 depicts A) Symptomatic profiles of the 4 ASD subtypes that resulted from K-means cluster analyses of 123 ADI-R severity scores per individual. In this figure, each row represents an individual and each column represents an item on the ADI-R. Black represents a score of 0 which is considered “normal”, while the intensity of red indicates severity scores ranging from 1-3. Gray represents missing data. The wide band of intensely red items in the language-impaired subgroup corresponds to spoken language. The 12 columns at the extreme right in each block correspond to “Savant skills”, which appear to be present at a slightly higher frequency in the group labeled “Moderate”. This group had been labeled “Savant” in our previous study (13). B) Principal components analysis (PCA) of the individuals based on the 123 ADI-R severity scores. Each subgroup of individuals identified in (A) is assigned a color, which indicates an individual from that subgroup in the PCA. Red: Language-impaired; Green: Intermediate; Yellow: Moderate; Blue: Mild. Each point on the PCA represents an individual with ASD whose position is defined by his/her scores for the 123 ADI-R items.

FIG. 6 depicts network connections centered on HTR4 from FIG. 3.

FIG. 7 depicts network connections centered on GCH1 from FIG. 3.

### DETAILED DESCRIPTION OF THE INVENTION

The invention disclosed herein provides methods and compositions for diagnosis and treatment of autism and autism spectrum disorder conditions. In particular, the invention provides biomarkers to diagnose and treat autism and autism spectrum disorders and to aid in the assessment or diagnosis of an individual's propensity or risk for having or developing an autism spectrum disorder. The invention relates, in part, to sets of genetic biomarkers that correlate with therapeutic treatments of neurological, and in particular, autism and autism spectrum disorders.

The invention provides not only methods of identifying biomarker profiles for autism and autism spectrum disorder conditions, but also methods of using such biomarker profiles in order to select particular therapeutic compounds useful in the prevention and treatment of such autism and autism spectrum disorder conditions. The invention further relates to the application of biomarker profiles for the identification of therapeutic targets, and related pharmaceutical methods and kits.

To provide an overall understanding of the invention, certain illustrative embodiments will now be described. However, it will be understood by one of ordinary skill in the art that the systems and methods described herein can be adapted and modified for other suitable applications and that such other additions and modifications will not depart from the scope hereof

### DEFINITIONS

For convenience, certain terms employed in the specification, examples, and appended claims, are collected here. Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article. By way of example, “an element” means one element or more than one element.

The term “including” is used herein to mean, and is used interchangeably with, the phrase “including but not limited to”.

The term “or” is used herein to mean, and is used interchangeably with, the term “and/or,” unless context clearly indicates otherwise.

The term “such as” is used herein to mean, and is used interchangeably, with the phrase “such as but not limited to”.

A “patient” or “subject” to be treated by the method of the invention can mean either a human or non-human animal, preferably a mammal.

The term “encoding” comprises an RNA product resulting from transcription of a DNA molecule, a protein resulting from the translation of an RNA molecule, or a protein resulting from the transcription of a DNA molecule and the subsequent translation of the RNA product.

The term “expression” is used herein to mean the process by which a polypeptide is produced from DNA. The process involves the transcription of the gene into mRNA and the translation of this mRNA into a polypeptide. Depending on the context in which used, “expression” may refer to the production of RNA, protein or both.

The term “transcriptional regulator” refers to a biochemical element that acts to prevent or inhibit the transcription of a promoter-driven DNA sequence under certain environmental conditions (e.g., a repressor or nuclear inhibitory protein), or to permit or stimulate the transcription of the promoter-driven DNA sequence under certain environmental conditions (e.g., an inducer or an enhancer).

The term “single nucleotide polymorphism (SNP)” refers to a change in which a single base in the DNA differs from the usual base at that position. These single base changes are called SNPs or “snips.” Millions of SNP's have been cataloged in the human genome. Some SNPs such as that which causes sickle cell are responsible for disease. Other SNPs are normal variations in the genome.

The terms “microarray,” “GeneChip,” “genome chip,” and “biochip,” as used herein refer to an ordered arrangement of hybridizeable array elements. The array elements are arranged so that there are preferably at least one or more different array elements on a substrate surface, such as paper, nylon or other type of membrane, filter, chip, glass slide, or any other suitable solid support. The hybridization signal from each of the array elements is individually distinguishable.

The terms “complementary” or “complementarity” as used herein refer to polynucleotides (i.e., a sequence of nucleotides) related by the base-pairing rules. For example, for the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods which depend upon binding between nucleic acids.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids.

As used herein, the term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxy ribonucleotide. The primer must be sufficiently long to prime the synthesis of extension products in the presence of the inducing agent. The exact lengths of the primers will depend on many factors, including temperature, source of primer and the use of the method.

As used herein, the term “probe” refers to an oligonucleotide (i.e., a sequence of nucleotides), whether occurring naturally as in a purified restriction digest or produced synthetically, recombinantly or by PCR amplification, which is capable of hybridizing to another oligonucleotide of interest. A probe may be single-stranded or double-stranded. Probes are useful in the detection, identification and isolation of particular gene sequences. It is contemplated that any probe used in the present invention will be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

As used herein, the terms “compound” and “test compound” refer to any chemical entity, pharmaceutical, drug, and the like that can be used to treat or prevent a disease, illness, conditions, or disorder of bodily function. Compounds comprise both known and potential therapeutic compounds. A compound can be determined to be therapeutic by screening using the screening methods of the present invention. A “known therapeutic compound” refers to a therapeutic compound that has been shown (e.g., through animal trials or prior experience with administration to humans) to be effective in such treatment. In other words, a known therapeutic compound is not limited to a compound efficacious in the treatment of cancer. Examples of test compounds include, but are not limited to peptides, polypeptides, synthetic organic molecules, naturally occurring organic molecules, nucleic acid molecules, and combinations thereof.

A “sample” from a subject may include a single cell or multiple cells or fragments of cells or an aliquot of body fluid, taken from the subject, by means including venipuncture, excretion, ejaculation, massage, biopsy, needle aspirate, lavage sample, scraping, surgical incision or intervention or other means known in the art.

As used herein, the term “subject” refers to a cell, tissue, or organism, human or non-human, whether in vivo, ex vivo or in vitro, under observation.

As used herein, the term “increased expression” refers to the level of a gene expression product that is made higher and/or the activity of the gene expression product that is enhanced. Preferably, the increase is by at least 1.22-fold, 1.5-fold, more preferably the increase is at least 2-fold, 5-fold, or 10-fold, and most preferably, the increase is at least 20-fold, relative to a control.

As used herein, the term “decreased expression” refers to the level of a gene expression product that is made lower and/or the activity of the gene expression product that is lowered. Preferably, the decrease is at least 25%, more preferably, the decrease is at least 50%, 60%, 70%, 80%, or 90% and most preferably, the decrease is at least one-fold, relative to a control.

As used herein, the term “gene profile” or “differentially expressed gene profile” refers to an experimentally verified subset of values associated with the expression level of a set of gene products from informative genes which allows the identification of a biological condition, an agent and/or its biological mechanism of action, or a physiological process.

As used herein, the term “differentially expressed gene profile,” or “gene expression profile” refers to the level or amount of gene expression of particular genes, for example, informative genes, as assessed by methods described herein. The differentially expressed gene expression profile or gene expression profile can comprise data for one or more informative genes and can be measured at a single time point or over a period of time. For example, the differentially expressed gene expression profile or gene expression profile can be determined using a single informative gene, or it can be determined using two or more informative genes, three or more informative genes, five or more informative genes, ten or more informative genes, twenty-five or more informative genes, or fifty or more informative genes. A differentially expressed gene expression profile or gene expression profile may include expression levels of genes that are not informative, as well as informative genes. Phenotype classification (e.g., the presence or absence of a autism or autism spectrum disorder) can be made by comparing the differentially expressed gene expression profile or gene expression profile of the sample with respect to one or more informative genes with one or more differentially expressed gene expression profile or gene expression profiles (e.g., in a database). Using the methods described herein, expression of numerous genes can be measured simultaneously. The assessment of numerous genes provides for a more accurate evaluation of the sample because there are more genes that can assist in classifying the sample. A differentially expressed gene expression profile or gene expression profile may involve only those genes that are increased in expression in a sample, only those genes that are decreased in expression in a sample, or a combination of genes that are increased and decreased in expression in a sample.

The terms “disorders” and “diseases” are used inclusively and refer to any deviation from the normal structure or function of any part, organ or system of the body (or any combination thereof). A specific disease is manifested by characteristic symptoms and signs, including biological, chemical and physical changes, and is often associated with a variety of other factors including, but not limited to, demographic, environmental, employment, genetic and medically historical factors. Certain characteristic signs, symptoms, and related factors can be quantitated through a variety of methods to yield important diagnostic information.

The term “neurological condition” or “neurological disorder” is used herein to mean mental, emotional, or behavioral abnormalities. These include but are not limited to autism spectrum disorder conditions including autism, asperger's disorder, bipolar disorder I or II, schizophrenia, schizoaffective disorder, psychosis, depression, stimulant abuse, alcoholism, panic disorder, generalized anxiety disorder, attention deficit disorder, post-traumatic stress disorder, Parkinson's disease, or a combination thereof.

Methods of Identifying Autism or Autism Spectrum Disorders Biomarkers

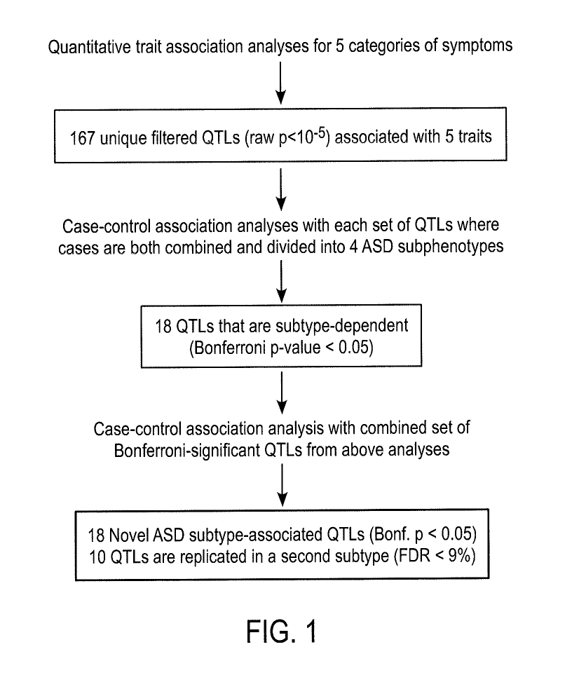

In the present invention a genome wide association mega analysis is provided that demonstrates that, in addition to multiple rare variations, part of the complex genetic architecture of autism involves certain common variation. Utilizing the compositions and methods disclosed herein certain biomarkers are identified as being associated with autism or autism spectrum disorders and include certain single nucleotide polymorphisms (SNPs) which demonstrated statistically significant strong association with autism and/or autism risk in both the discovery and validation datasets. These findings further support this stepwise approach as depicted in FIG. 1 of first delineating the heterogeneity of autism before applying genetic association analyses.

The methodology for identifying biomarkers for the diagnosis of autism and autism spectrum disorders is more particularly described in the Examples et seq, infra.

In particular, in one aspect of the present invention, a method is provided for identifying biomarkers for the diagnosis of autism and autism spectrum disorders comprising (a) performing quantitative trait association analysis for at least one category of symptoms or related quantitative traits, to identify filtered set of single nucleotide polymorphisms that are associated with each quantitative trait; (b) performing case-control association analysis with each set of trait-associated single nucleotide polymorphisms in which cases are both combined and divided into from at least one to at least four ASD subtypes to identify trait associated single nucleotide polymorphisms that are subtype-dependent with a Bonferroni significance of P<0.05; (c) performing case control association analysis with the combined set of Bonferroni significant single nucleotide polymorphisms from analysis in step (b) to identify those novel ASD subtype-associated single nucleotide polymorphisms that are associated with each quantitative trait and those novel ASD subtype-associated quantitative trait loci that are replicated in a second subtype.

In one embodiment of the present invention, quantitative severity criteria are assessed across at least one category of behavioral symptoms or quantitative traits of ASD subtypes comprising language deficits, deficits in nonverbal communication, under developed playful skills, delayed social development, and sensory issues/stereotypes, or any combination thereof.

In one embodiment of the method of the present invention, the samples are assessed in a genome-wide association analysis (GWAS).

In one embodiment of the present invention, quantitative severity criteria are assessed across at least one category of behavioral symptoms or quantitative traits of ASD subtypes comprising language deficits, deficits in nonverbal communication, under developed playful skills, delayed social development, and sensory issues/stereotypes, separately or in combination with measuring the level of differential gene expression in one or more of the biomarker-associated genes listed in Table 1 or Table 7, or any combination thereof.

In one embodiment of the present invention, wherein the case-control association analysis of step (b) comprises a cluster analysis to divide the autistic cases into four phenotypic subgroups according to symptomatic severity profiles derived from the one to one hundred and twenty three items listed on the ADI-R assessments in Table 1 to reduce the behavioral/symptomatic and heterogeneity genetic heterogeneity among the cases within each subgroup.

In one embodiment of the cluster analysis of the case-control association analysis of step (b), the ADI-R assessments comprise items one to one hundred and twenty three (123), or any integer value there between of the published ADI-R assessments as described in Hu V W & Steinberg M E (2009) Novel clustering of items from the autism diagnostic interview-revised to define phenotypes within autism spectrum disorders. Autism Res 2: 67-77, incorporated by reference herein in its entirety.

In one embodiment of the present invention, the four phenotypic subgroups obtained from the cluster analysis distinguish between different variants of autism spectrum disorder comprising a “mild” subgroup with lower severity scores across all ADIR items, a subgroup with intermediate severity across all ADIR items, a severely language-impaired subgroup with higher severity scores on spoken language items on the ADIR, a subgroup with a moderate severity profile, often with higher frequency of savant skills, or any combination thereof.

In one embodiment of the method of the present invention, the samples from families with Mendelian errors greater than 2% are excluded.

In another embodiment of the method of the present invention, single nucleotide polymorphisms having a Hardy-Weinberg equilibrium (HWE) p-value of about less than 10<˜6> and a Mendelian Error (ME) of greater than about 4% are excluded.

In certain embodiments of the method of the present invention, the novel ASD subtype-associated single nucleotide polymorphisms that are associated with each quantitative trait and/or those novel ASD subtype-associated quantitative trait loci that are replicated in a second subtype ASD subtyping method either specifically exclude or specifically include those single nucleotide polymorphisms selected from the group consisting of rs4307059, rs7704909, rs12518194, rs4327572, rs1896731, and rs10038113 (Wang K, et al (2009) Common genetic variants on 5p14.1 associate with autism spectrum disorders. Nature 459: 528-533; Ma D, et al (2009), incorporated by reference herein in its entirety) or variants, mutants, alleles or complementary sequences thereof, or any combination thereof.

Biomarkers for Autism or Autism Spectrum Disorders

By using the aforementioned method of identifying biomarkers associated with autism or autism spectrum disorders, certain single nucleotide polymorphisms (SNPs) biomarkers were identified that are associated with ASD subtype and which may therefore be used as novel biomarkers for autism. Furthermore, since these single nucleotide polymorphisms biomarkers are dependent upon the subtype (phenotype) of autism spectrum disorders, they may also be useful for identifying the subtypes of ASD which may respond to different therapies (i.e., pharmacogenomics).

In particular, in one aspect of the present invention, a biomarker specifically identified using the above-identified method is provided for the diagnosis of autism and autism spectrum disorders comprising at least one language impairment quantitative trait loci-specific single nucleotide polymorphism, at least one non-verbal communication quantitative trait loci-specific single nucleotide polymorphism, at least one play skills quantitative trait loci-specific single nucleotide polymorphism, at least one insistence on sameness/rituals quantitative trait loci-specific single nucleotide polymorphism, and/or at least one social skills and development quantitative trait loci-specific single nucleotide polymorphism, or variants, mutants, alleles or complementary sequences thereof, or any combination thereof.

In one embodiment of the present invention, a biomarker specifically identified using the above-identified method is thus provided for the diagnosis of autism spectrum disorders comprising at least one language impairment quantitative trait loci-specific single nucleotide polymorphism, at least one non-verbal communication quantitative trait loci-specific single nucleotide polymorphism, at least one play skills quantitative trait loci-specific single nucleotide polymorphism, at least one insistence on sameness/rituals quantitative trait loci-specific single nucleotide polymorphism, and/or at least one social skills and development quantitative trait loci-specific single nucleotide polymorphism comprising a biomarker set forth as in Table 1, variants, mutants, alleles or complementary sequences thereof, or any combination thereof.

Accordingly, in one embodiment of the invention, a biomarker specifically identified using the above-identified method is provided for the diagnosis of autism spectrum disorders comprising i) at least one language impairment quantitative trait loci-specific single nucleotide polymorphism set forth as: rs12407665, rs17828521, rs9474831, rs6454792, rs10183984, rs11969265, rs1231339, rs10806416, rs7785107, rs2277049, rs757099, rs7725785, rs758158, rs2287581, rs17830215, rs2180055, rs12893752; ii) at least one non-verbal communication quantitative trait loci-specific single nucleotide polymorphism set forth as: rs9941626, rs13205238, rs11671930, rs11229410, rs11229413, rs11229411, rs11721070, rs12466917, rs13076171, rs7930778, rs12962411, rs12279895, rs730168, rs13021324, rs564127, rs1231339, rs393076, rs1938651, rs11138895, rs1938672, rs4804202, rs665036, rs4527692, rs519514, rs3133855, rs1938670; iii) at least one play skills quantitative trait loci-specific single nucleotide polymorphism set forth as: rs13205238, rs1996893, rs12606567, rs3769845, rs2422675, rs4798405, rs10040891, rs8181738, rs11950809, rs11627027, rs1930, rs4894734, rs1482930, rs11671930, rs4980777, rs1481513, rs10987251, rs2151206, rs2044747, rs1440423, rs4745257, rs2779499, rs1796028, rs1888156, rs6734788, rs7605424, rs4627775, rs5009527, rs1796045, rs1863080, rs7337921, rs6452136, rs2168709, rs4386512, rs12614870, rs10491885, rs4646421, rs4894733, rs7944323, rs6791089, rs11229410, rs17770167, rs6698676, rs11664663, rs6482516, rs11082277, rs6988293, rs6974649, rs730168, rs1461710, rs9941626, rs3745651, rs9536962, rs7529505, rs9342127, rs1554547, rs9508456, rs2078520, rs9569991, rs3825597, rs3754741, rs2250595, rs1055518, rs2600685; at least one insistence on sameness/rituals quantitative trait loci-specific single nucleotide polymorphism set forth as: rs164187, rs3809854, rs3804967, rs3804968, rs317985, rs9634811, rs7819605, rs7950390, rs4436186, rs4838964, rs1827924, rs7699496, rs3861787, rs6782718, rs11038286, rs693442, rs1452885, rs17599556, rs185425, rs11035240, rs9693369, rs10781238, rs9568011, rs11682846, rs7650071, rs2574852, rs11914753, rs2469183, rs274646, rs13096022, rs17738966, rs6461176; at least one social skills and development quantitative trait loci-specific single nucleotide polymorphism set forth as: rs13205238, rs11138895, rs4809918, rs9479482, rs1294264, rs10788819, rs4959923, rs4905110, rs721087, rs12266938, rs10874468, rs13384439, rs4416176, rs10519124, rs12962411, rs6022029, rs11627027, rs6022039, rs10886048, rs4873815, rs4832481, rs3809282, rs1554547, rs2297172, rs2255313, rs2627468, rs12183587, rs10305860, rs30746, rs11138885, rs1294293, rs12115722, rs6698676, rs10997162, rs4646421, rs4778640, rs10110252, rs1996893, rs12811136, rs17192980, rs4811895, rs2519866, rs2779499, or rs2151206, or variants, mutants, alleles or complementary sequences thereof, or any combination thereof.

In yet another aspect of the present invention, a biomarker specifically identified using the above-identified method is provided for the diagnosis of autism and autism spectrum disorders comprising at least one combined quantitative trait loci-specific and ASD sub-type language impaired-specific single nucleotide polymorphism, at least one combined quantitative trait loci-specific and ASD sub-type intermediate-specific single nucleotide polymorphism, at least one combined quantitative trait loci-specific and ASD sub-type moderate-specific single nucleotide polymorphism, or at least one combined quantitative trait loci-specific and ASD sub-type mild-specific single nucleotide polymorphism, or variants, mutants, alleles or complementary sequences thereof, or any combination thereof.

In one embodiment of the present invention, a biomarker specifically identified using the above-identified method is provided for the diagnosis of autism and autism spectrum disorders comprising at least one combined quantitative trait loci-specific and ASD sub-type specific single nucleotide polymorphism set forth as rs2277049, rs757099, rs7785107, rs7725785, rs2287581, rs1231339, rs2180055, rs11671930, rs7950390, rs12266938, rs3861787, rs1827924, rs17738966, rs317985, rs730168, rs10519124, rs6482516, or rs2297172; at least one combined quantitative trait loci-specific and ASD sub-type language impaired-specific single nucleotide polymorphism set forth as: rs2277049, rs757099, rs7785107, rs7725785, rs2287581, rs1231339, rs2180055, rs11671930, at least one combined quantitative trait loci-specific and ASD sub-type intermediate-specific single nucleotide polymorphism set forth as: rs7785107, rs7950390, rs12266938, rs3861787, at least one combined quantitative trait loci-specific and ASD sub-type moderate-specific single nucleotide polymorphism set forth as: rs1827924, rs17738966, rs7950390, rs3861787, rs317985, at least one combined quantitative trait loci-specific and ASD sub-type mild-specific single nucleotide polymorphism set forth as: rs12266938, rs730168, rs10519124, rs6482516, rs11671930, rs2297172, rs317985, rs1827924, rs1231339, rs757099, rs7725785, at least one combined quantitative trait loci-specific and ASD sub-type language impaired and ASD sub-type moderate and ASD subtype mild-specific single nucleotide polymorphism set forth as: rs1827924, rs17738966, rs7950390, rs3861787, rs317985, rs12266938, rs730168, rs10519124, rs6482516, rs11671930, rs2297172, rs317985, rs1827924, rs1231339, rs757099, rs7725785, or variants, mutants, alleles or complementary sequences thereof, or any combination thereof.

In yet another embodiment of the present invention, a biomarker is provided for the diagnosis of autism spectrum disorders comprising at least one combined quantitative trait loci-specific and ASD sub-type language impaired-specific single nucleotide polymorphism set forth as: rs2277049, rs7725785, rs2287581, or rs11671930 (associated with HTR4, a significantly differentially expressed gene by large-scale gene expression profiling of subtypes of ASD (FDR<5%); at least one combined quantitative trait loci-specific and ASD sub-type intermediate-specific single nucleotide polymorphism set forth as: rs7950390 (associated with TTRIM68, a significantly differentially expressed gene by large-scale gene expression profiling of subtypes of ASD (FDR<5%); at least one combined quantitative trait loci-specific and ASD sub-type moderate-specific single nucleotide polymorphism set forth as: rs1827924 (associated with CCL20, a differentially expressed gene by large-scale gene expression profiling of subtypes of ASD (FDR<5%), rs17738966 (associated with GCH1, a differentially expressed gene by large-scale gene expression profiling of subtypes of ASD (FDR<5%), rs7950390 (associated with TRIM68, a differentially expressed gene by large-scale gene expression profiling of subtypes of ASD (FDR<5%), rs77255785 (associated with HTR4, a differentially expressed gene by large-scale gene expression profiling of subtypes of ASD (FDR<5%), at least one combined quantitative trait loci-specific and ASD sub-type mild-specific single nucleotide polymorphism set forth as: rs730168 (associated with LDHD, a differentially expressed gene by large-scale gene expression profiling of subtypes of ASD (FDR<5%), rs6482516 (associated with LDHD, a differentially expressed gene by large-scale gene expression profiling of subtypes of ASD (FDR<5%), rs11671930 (associated with CCL25, a differentially expressed gene by large-scale gene expression profiling of subtypes of ASD (FDR<5%), rs2297172 (associated with PTAR1, a differentially expressed gene by large-scale gene expression profiling of subtypes of ASD (FDR<5%), rs1827924 (associated with CCL20, a differentially expressed gene by large-scale gene expression profiling of subtypes of ASD (FDR<5%), rs7725785 (associated with HTR4, a differentially expressed gene by large-scale gene expression profiling of subtypes of ASD (FDR<5%), variants, mutants, alleles or complementary sequences thereof, or any combination thereof. The correlation of differentially expressed genes with those associated with at least some of the novel single nucleotide polymorphisms lends support to the functional relevance of these mainly intronic, promoter, or downstream-specific single nucleotide polymorphisms to ASD.

In one embodiment of each of the methods of the present invention, the use of the biomarkers of Table 1, Table 7, or Table 8 either specifically excludes or specifically includes those single nucleotide polymorphism biomarkers associated with a cadherin gene [(cadherin gene 10 (CDH1O) and cadherin gene 9 (CDH9)] and/or protocadherin gene.

In one embodiment of each of the methods of the present invention, the novel ASD subtype-associated single nucleotide polymorphisms that are associated with each quantitative trait and/or those novel ASD subtype-associated quantitative trait loci that are replicated in a second subtype ASD subtyping method either specifically exclude or specifically include those single nucleotide polymorphisms selected from the group consisting of rs4307059, rs7704909, rs12518194, rs4327572, rs1896731, and rs10038113 (Wang K, et al (2009) Common genetic variants on 5p14.1 associate with autism spectrum disorders. Nature 459: 528-533 incorporated in its entirety herein by reference) or variants, mutants, alleles or complementary sequences thereof, or any combination thereof.

Biomarker Gene Chips

In the methods described herein, the detection of a biomarker in a subject can be carried out according to various methods well known in the art. For example DNA is obtained from any suitable sample from the subject that will contain DNA, genomic DNA, and the DNA is then prepared and analyzed according to well-established protocols for the presence of biomarkers according to the methods of this invention. In some embodiments, analysis of the DNA can be carried out by amplification of the region of interest according to amplification protocols well known in the art (e.g., polymerase chain reaction, ligase chain reaction, strand displacement amplification, transcription-based amplification, self-sustained sequence replication (3SR), Q-Beta replicase protocols, nucleic acid sequence-based amplification (NASBA), repair chain reaction (RCR) and boomerang DNA amplification (BDA)). The amplification product can then be visualized directly in a gel by staining or the product can be detected by hybridization with a detectable probe. When amplification conditions allow for amplification of all allelic types of a biomarker, the types can be distinguished by a variety of well-known methods, such as hybridization with an allele-specific probe, secondary amplification with allele-specific primers, by restriction endonuclease digestion, or by electrophoresis. Thus, the present invention can further provide oligonucleotides for use as primers and/or probes for detecting and/or identifying biomarkers according to the methods of this invention. These biomarker specific probes can then be used in microarrays. By way of example, and not by way of limitation, the use of the biomarkers as described herein on microarrays to diagnose, screen and/or predict for the risk of autism or ASD is explained in detail infra.

Accordingly, one aspect of the invention provides gene chips specific for one or more of the biomarkers identified using the methods of the present invention. Gene chips, also called “biochips” or “arrays” or “microarrays” are miniaturized devices typically with dimensions in the micrometer to millimeter range for performing chemical and biochemical reactions and are particularly suited for embodiments of the invention. Arrays may be constructed via microelectronic and/or microfabrication using essentially any and all techniques known and available in the semiconductor industry and/or in the biochemistry industry, provided that such techniques are amenable to and compatible with the deposition and screening of polynucleotide sequences. Microarrays are particularly desirable for their virtues of high sample throughput and low cost for generating profiles and other data.

Accordingly, in one embodiment of the present invention, a microarray is provided having a plurality of different oligonucleotides with specificity for at least one single nucleotide polymorphism set forth in Table 2, or variants, mutants, alleles or complementary sequences thereof, or a combination thereof which are associated with at least one autism spectrum disorder, wherein the autism spectrum disorder comprises autistic disorder, pervasive developmental disorder-not otherwise specified (PDD-NOS), including atypical autism, Asperger's Disorder, or a combination thereof.

In another embodiment of the present invention, a microarray having a plurality of different oligonucleotides with specificity for at least one single nucleotide polymorphism set forth as rs2277049, rs757099, rs7785107, rs7725785, rs2287581, rs1231339, rs2180055, rs11671930, rs7950390, rs12266938, rs3861787, rs1827924, rs17738966, rs317985, rs730168, rs10519124, rs6482516, or rs2297172, or variants, mutants, alleles or complementary sequences thereof, or any combination thereof, which are associated with at least one autism spectrum disorder, wherein the autism spectrum disorder comprises autistic disorder, pervasive developmental disorder-not otherwise specified (PDD-NOS), including atypical autism, Asperger's Disorder, or a combination thereof.

In another specific embodiment of the gene chips provided herein, the gene chip comprises at least 3, 5, 10, 15, 20 or 25 of the probes are derived from oligonucleotides that are specific for the single nucleotide polymorphism biomarkers set out in Tables 1, 2, 3, 4, 5, 6, 7, 8, or a combination thereof. In a related embodiment, at least 50% of the probes on the gene chip are derived from oligonucleotides that are specific for the single nucleotide polymorphism biomarkers set out in Tables 1, 2, 3, 4, 5, 6, 7, 8, or a combination thereof. In a related embodiment, at least 70%, 80%, 90%, 95% or 98% of the probes on the gene chip are derived from oligonucleotides that are specific for the single nucleotide polymorphism biomarkers set out in Tables 1, 2, 3, 4, 5, 6, 7, 8, or combinations thereof.

DNA microarray and methods of analyzing data from microarrays are well-described in the art, including in DNA Microarrays: A Molecular Cloning Manual, Ed by Bowtel and Sambrook (Cold Spring Harbor Laboratory Press, 2002); Microarrays for an Integrative Genomics by Kohana (MIT Press, 2002); A Biologist's Guide to Analysis of DNA Microarray Data, by Knudsen (Wiley, John & Sons, Incorporated, 2002); and DNA Microarrays: A Practical Approach, Vol. 205 by Schema (Oxford University Press, 1999); and Methods of Microarray Data Analysis II, ed by Lin et ad al. (Kluwer Academic Publishers, 2002), hereby incorporated by reference in their entirety.

Microarrays may be prepared by selecting probes which comprise a polynucleotide sequence, and then immobilizing such probes to a solid support or surface. For example, the probes may comprise DNA sequences, RNA sequences, or copolymer sequences of DNA and RNA. The polynucleotide sequences of the probes may also comprise DNA and/or RNA analogues, or combinations thereof. For example, the polynucleotide sequences of the probes may be full or partial fragments of genomic DNA. The polynucleotide sequences of the probes may also be synthesized nucleotide sequences, such as synthetic oligonucleotide sequences. The probe sequences can be synthesized either enzymatically in vivo, enzymatically in vitro (e.g., by PCR), or non-enzymatically in vitro.

The probe or probes used in the methods and gene chips of the invention may be immobilized to a solid support which may be either porous or non-porous. For example, the probes of the invention may be polynucleotide sequences which are attached to a nitrocellulose or nylon membrane or filter covalently at either the 3′ or the 5′ end of the polynucleotide. Such hybridization probes are well known in the art (see, e.g., Sambrook et al. et al., MOLECULAR CLONING—A LABORATORY MANUAL (2ND ED.), Vols. 1-3, Cold Spring Harbor Laboratory, Cold Spring Harbor, N.Y. (1989). Alternatively, the solid support or surface may be a glass or plastic surface. In one embodiment, hybridization levels are measured to microarrays of probes consisting of a solid phase on the surface of which are immobilized a population of polynucleotides, such as a population of DNA or DNA mimics, or, alternatively, a population of RNA or RNA mimics. The solid phase may be a nonporous or, optionally, a porous material such as a gel.

In one embodiment, a microarray comprises a support or surface with an ordered array of binding (e.g., hybridization) sites or “probes” each representing one of the markers described herein. Preferably the microarrays are addressable arrays, and more preferably positionally addressable arrays. More specifically, each probe of the array is preferably located at a known, predetermined position on the solid support such that the identity (i.e., the sequence) of each probe can be determined from its position in the array (i.e., on the support or surface). In preferred embodiments, each probe is covalently attached to the solid support at a single site.

Microarrays can be made in a number of ways, of which several are described below. However produced, microarrays share certain characteristics. The arrays are reproducible, allowing multiple copies of a given array to be produced and easily compared with each other. Preferably, microarrays are made from materials that are stable under binding (e.g., nucleic acid hybridization) conditions. The microarrays are preferably small, e.g., between 1 cm² and 25 cm², between 12 cm² and 13 cm², or about 3 cm². However, larger arrays are also contemplated and may be preferable, e.g., for use in screening arrays. Preferably, a given binding site or unique set of binding sites in the microarray will specifically bind (e.g., hybridize) to the product of a single gene in a cell (e.g., to a specific mRNA). However, in general, other related or similar sequences will cross hybridize to a given binding site.

The microarrays of the present invention include one or more test probes, each of which has a polynucleotide sequence that is complementary to a subsequence of RNA or DNA to be detected. Preferably, the position of each probe on the solid surface is known. Indeed, the microarrays are preferably positionally addressable arrays. Specifically, each probe of the array is preferably located at a known, predetermined position on the solid support such that the identity (i.e., the sequence) of each probe can be determined from its position on the array (i.e., on the support or surface).

According to one aspect of the invention, the microarray is an array (i.e., a matrix) in which each position represents one of the biomarkers as described herein. For example, each position can contain a DNA or DNA analogue based on genomic DNA to which a particular RNA transcribed from that biomarker can specifically hybridize. The DNA or DNA analogue can be, for example, a synthetic oligomer or a gene fragment. In one embodiment, probes representing each of the single nucleotide polymorphism biomarkers set out in Tables 1, 2, 3, 4, 5, 6, 7, 8, or a combination thereof are present on the array.

As noted above, the “probe” to which a particular polynucleotide molecule specifically hybridizes according to the invention contains a complementary polynucleotide sequence. In one embodiment, the probes of the single nucleotide polymorphism biomarkers set out in Tables 1, 2, 3, 4, 5, 6, 7, 8, or a combination thereof consist of nucleotide sequences of 10 to 1,000 nucleotides. In a preferred embodiment, the nucleotide sequences of the probes are in the range of 10-200 nucleotides in length and are genomic sequences of a species of organism, such that a plurality of different probes is present, with sequences complementary and thus capable of hybridizing to the genome of such a species of organism, sequentially tiled across all or a portion of such genome. In other specific embodiments, the probes are in the range of 10-30 nucleotides in length, in the range of 10-40 nucleotides in length, in the range of 20-50 nucleotides in length, in the range of 40-80 nucleotides in length, in the range of 50-150 nucleotides in length, in the range of 80-120 nucleotides in length, and most preferably are 60 nucleotides in length.

The probes may comprise DNA or DNA “mimics” (e.g., derivatives and analogues) corresponding to a portion of an organism's genome. In another embodiment, the probes of the microarray are complementary RNA or RNA mimics. DNA mimics are polymers composed of subunits capable of specific, Watson-Crick-like hybridization with DNA, or of specific hybridization with RNA. The nucleic acids can be modified at the base moiety, at the sugar moiety, or at the phosphate backbone. Exemplary DNA mimics include, e.g., phosphorothioates.

DNA can be obtained, e.g., by polymerase chain reaction (PCR) amplification of genomic DNA or cloned sequences. PCR primers are preferably chosen based on a known sequence of the genome that will result in amplification of specific fragments of genomic DNA. Computer programs that are well known in the art are useful in the design of primers with the required specificity and optimal amplification properties, such as Oligo version 5.0 (National Biosciences). Typically each probe on the microarray will be between 10 bases and 50,000 bases, usually between 300 bases and 1,000 bases in length. PCR methods are well known in the art, and are described, for example, in Innis et al. et al., eds., PCR: Protocols: A Guide to Methods and Applications, Academic Press Inc., San Diego, Calif. (1990). It will be apparent to one skilled in the art that controlled robotic systems are useful for isolating and amplifying nucleic acids.

An alternative, means for generating the polynucleotide probes of the microarray is by synthesis of synthetic polynucleotides or oligonucleotides, e.g., using N-phosphonate or phosphoramidite chemistries (Froehler et al. et al., Nucleic Acid Res. 14:5399-5407 (1986); McBride et al. et al., Tetrahedron Lett. 24:246-248 (1983)). Synthetic sequences are typically between about 10 and about 500 bases in length, more typically between about 20 and about 100 bases, and most preferably between about 40 and about 70 bases in length. In some embodiments, synthetic nucleic acids include non-natural bases, such as, but by no means limited to, inosine. As noted above, nucleic acid analogues may be used as binding sites for hybridization. An example of a suitable nucleic acid analogue is peptide nucleic acid (see, e.g., Egholm et al. et al., Nature 363:566-568 (1993); U.S. Pat. No. 5,539,083). Probes are preferably selected using an algorithm that takes into account binding energies, base composition, sequence complexity, cross-hybridization binding energies, and secondary structure (see Friend et al., International Patent Publication WO 01/05935, published Jan. 25, 2001; Hughes et al., Nat. Biotech. 19:342-7 (2001)).

A skilled artisan will also appreciate that positive control probes, e.g., probes known to be complementary and hybridizable to sequences in the DNA molecules, and negative control probes, e.g., probes known to not be complementary and hybridizable to sequences in the DNA molecules, should be included on the array. In one embodiment, positive controls are synthesized along the perimeter of the array. In another embodiment, positive controls are synthesized in diagonal stripes across the array. In still another embodiment, the reverse complement for each probe is synthesized next to the position of the probe to serve as a negative control. In yet another embodiment, sequences from other species of organism are used as negative controls or as “spike-in” controls.

The probes may be attached to a solid support or surface, which may be made, e.g., from glass, plastic (e.g., polypropylene, nylon), polyacrylamide, nitrocellulose, gel, or other porous or nonporous material. A preferred method for attaching the nucleic acids to a surface is by printing on glass plates, as is described generally by Schena et al, Science 270:467-470 (1995). This method is especially useful for preparing microarrays of cDNA (See also, DeRisi et al, Nature Genetics 14:457-460 (1996); Shalon et al., Genome Res. 6:639-645 (1996); and Schena et al., Proc. Natl. Acad. Sci. U.S.A. 93:10539-11286 (1995)).

Additional Methods of Use for Biomarkers

In another aspect of the present invention, the single nucleotide polymorphism biomarkers identified using the methods described supra may be used to, for example, and not by way of limitation, diagnose, to treat and/or to screen for the presence of autism or autism spectrum disorders.

Thus, in one aspect of the present invention, a method is provided for identifying a biomarker for the diagnosis of autism and autism spectrum disorders comprising obtaining a sample from individuals and their families and purifying genomic DNA from the sample; genotyping single nucleotide polymorphisms (SNP); assessing the single nucleotide polymorphisms; and, identifying a biomarker for the diagnosis of autism and autism spectrum disorders.

Accordingly, in one embodiment aspect of the present invention, a method is provided for diagnosing a patient with autism or autism spectrum disorder comprising identifying in a patient a biomarker or biomarker set comprising at least one single nucleotide polymorphism set forth in Tables 2, 3, 4, 5, 6, 7, or 8 or set forth as rs2277049, rs757099, rs7785107, rs7725785, rs2287581, rs1231339, rs2180055, rs11671930, rs7950390, rs12266938, rs3861787, rs1827924, rs17738966, rs317985, rs730168, rs10519124, rs6482516, or rs2297172, or variants, mutants, alleles or complementary sequences thereof, or any combination thereof; and, diagnosing a patient with autism or autism spectrum disorder.

In one embodiment of the present invention, a method is provided for diagnosing a patient pre-natally or post-natally with an autism spectrum disorder comprising detecting at least one single nucleotide polymorphism set forth in Tables 2, 3, 4, 5, 6, 7, or 8 or as set forth as rs2277049, rs757099, rs7785107, rs7725785, rs2287581, rs1231339, rs2180055, rs11671930, rs7950390, rs12266938, rs3861787, rs1827924, rs17738966, rs317985, rs730168, rs10519124, rs6482516, or rs2297172, or variants, mutants, alleles or complementary sequences thereof, or any combination thereof; and, diagnosing a patient with autism or autism spectrum disorder.

In various embodiments of the diagnosing methods of the present invention, the autism spectrum disorder comprises autistic disorder, pervasive developmental disorder-not otherwise specified (PDD-NOS), including atypical autism, Asperger's Disorder, or a combination thereof.

In another aspect of the invention, a method is provided for detecting a propensity for developing autism or autistic spectrum disorder in a patient in need thereof.

Accordingly, in one embodiment of the invention, a screening method is provided for detecting in a subject in need thereof a propensity or increased risk for developing an autism spectrum disorder that entails detecting the presence of at least one single nucleotide polymorphism in a target polynucleotide(s) wherein if said at least one single nucleotide polymorphism is present, said subject has an increased risk for developing autism and/or autistic spectrum disorder, wherein said single nucleotide polymorphism comprises a single nucleotide polymorphism set forth in Tables 2, 3, 4, 5, 6, 7, or 8 or set forth as rs2277049, rs757099, rs7785107, rs7725785, rs2287581, rs1231339, rs2180055, rs11671930, rs7950390, rs12266938, rs3861787, rs1827924, rs17738966, rs317985, rs730168, rs10519124, rs6482516, or rs2297172, or variants, mutants, alleles or complementary sequences thereof, or any combination thereof, e.g., (a) rs2277049, rs757099, rs7785107, rs7725785, rs2287581, rs1231339, rs2180055, and rs11671930, which are all associated with the language impairment subtype; (b) rs7785107, rs7950390, rs12266938, and rs3861787, which are all associated with the intermediate subtype; (c) rs1827924, rs17738966, rs11671930, rs3861787, and rs317985 which are all associated with the moderate subtype, and (d) rs12266938, rs730168, rs10519124, rs6482516, rs11671930, rs2297172, rs317985, rs1827924, rs1231339, rs757099, and rs7725785, which are all associated with the mild subtype as set forth in Table 7. In an embodiment of the invention the likelihood of a subject having a propensity or risk for developing an autism spectrum disorder increases as the number of SNPs described herein that are present in the subject increases.

In one embodiment of the screening method, the autism spectrum disorder comprises autistic disorder, pervasive developmental disorder-not otherwise specified (PDD-NOS), including atypical autism, Asperger's Disorder, or a combination thereof.

In one embodiment, the invention also provides at least one isolated autism-related SNP-containing nucleic acid identified using the aforementioned screening method wherein the autism-related SNP-containing nucleic acid is selected from the group consisting of rs2277049, rs757099, rs7785107, rs7725785, rs2287581, rs1231339, rs2180055, rs11671930, rs7950390, rs12266938, rs3861787, rs1827924, rs17738966, rs317985, rs730168, rs10519124, rs6482516, or rs2297172, or variants, mutants, alleles or complementary sequences thereof, or any combination thereof.

In another aspect, the present invention also provides for expression of SNP-containing nucleic acids exemplified in set forth in Tables 2, 3, 4, 5, 6, 7, or 8 or variants, mutants, alleles or complementary sequences thereof, or any combination thereof that may optionally be contained in a suitable expression vector.

An expression vector is a recombinant polynucleotide that is in chemical form either a deoxyribonucleic acid (DNA) and/or a ribonucleic acid (RNA). The physical form of the expression vector may also vary in strandedness (e.g., single-stranded or double-stranded) and topology (e.g., linear or circular). The expression vector is preferably a double-stranded deoxyribonucleic acid (dsDNA) or is converted into a dsDNA after introduction into a cell (e.g., insertion of a retrovirus into a host genome as a provirus). The expression vector may include one or more regions from a mammalian gene expressed in the microvasculature, especially endothelial cells (e.g., ICAM-2, tie), or a virus (e.g., adenovirus, adeno-associated virus, cytomegalovirus, fowlpox virus, herpes simplex virus, lentivirus, Moloney leukemia virus, mouse mammary tumor virus, Rous sarcoma virus, SV40 virus, vaccinia virus), as well as regions suitable for genetic manipulation (e.g., selectable marker, linker with multiple recognition sites for restriction endonucleases, promoter for in vitro transcription, primer annealing sites for in vitro replication). The expression vector may be associated with proteins and other nucleic acids in a carrier (e.g., packaged in a viral particle) or condensed with chemicals (e.g., cationic polymers) to target entry into a cell or tissue.

The expression vector further comprises a regulatory region for gene expression (e.g., promoter, enhancer, silencer, splice donor and acceptor sites, polyadenylation signal, cellular localization sequence). Transcription can be regulated by tetracyline or dimerized macrolides. The expression vector may be further comprised of one or more splice donor and acceptor sites within an expressed region; Kozak consensus sequence upstream of an expressed region for initiation of translation; and downstream of an expressed region, multiple stop codons in the three forward reading frames to ensure termination of translation, one or more mRNA degradation signals, a termination of transcription signal, a polyadenylation signal, and a 3′ cleavage signal. For expressed regions that do not contain an intron (e.g., a coding region from a cDNA), a pair of splice donor and acceptor sites may or may not be preferred. It would be useful, however, to include mRNA degradation signal(s) if it is desired to express one or more of the downstream regions only under the inducing condition. An origin of replication may also be included that allows replication of the expression vector integrated in the host genome or as an autonomously replicating episome. Centromere and telomere sequences can also be included for the purposes of chromosomal segregation and protecting chromosomal ends from shortening, respectively. Random or targeted integration into the host genome is more likely to ensure maintenance of the expression vector but episomes could be maintained by selective pressure or, alternatively, may be preferred for those applications in which the expression vector is present only transiently.

An expressed region may be derived from any gene of interest, and be provided in either orientation with respect to the promoter; the expressed region in the antisense orientation will be useful for making cRNA and antisense polynucleotide. The gene may be derived from the host cell or organism, from the same species thereof, or designed de novo; but it is preferably of archael, bacterial, fungal, plant, or animal origin. The gene may have a physiological function of one or more nonexclusive classes: axon guidance, synaptic transmission or plasticity, myelination, long-term potentiation, neuron toxicity, embryonic development, regulation of actin networks, KEGG pathway, digestion, liver toxicity (hepatic stellate cell activation, fibrosis, and cholestasis), inflammation, oxidative stress, epilepsy, apoptosis, cell survival, differentiation, the unfolded protein response, Type II diabetes and insulin signaling, endocrine function, circadian rhythm, cholesterol metabolism and the steroidogenesis pathway, adhesion proteins; steroids, cytokines, hormones, and other regulators of cell growth, mitosis, meiosis, apoptosis, differentiation, circadian rthym, or development; soluble or membrane receptors for such factors; adhesion molecules; cell-surface receptors and ligands thereof; cytoskeletal and extracellular matrix proteins; cluster differentiation (CD) antigens, antibody and T-cell antigen receptor chains, histocompatibility antigens, and other factors mediating specific recognition in immunity; chemokines, receptors thereof, and other factors involved in inflammation; enzymes producing lipid mediators of inflammation and regulators thereof; clotting and complement factors; ion channels and pumps; transporters and binding proteins; neurotransmitters, neurotrophic factors, and receptors thereof; cell cycle regulators, oncogenes, and tumor suppressors; other transducers or components of signaling pathways; proteases and inhibitors thereof; catabolic or metabolic enzymes, and regulators thereof. Some genes produce alternative transcripts, encode subunits that are assembled as homopolymers or heteropolymers, or produce propeptides that are activated by protease cleavage. The expressed region may encode a translational fusion; open reading frames of the regions encoding a polypeptide and at least one heterologous domain may be ligated in register. If a reporter or selectable marker is used as the heterologous domain, then expression of the fusion protein may be readily assayed or localized. The heterologous domain may be an affinity or epitope tag.

In yet another aspect of the present invention, an in vitro diagnostic test is provided for diagnosing, predicting, or assessing a propensity or increased risk of developing ASD in an individual, the in vitro diagnostic test comprising at least one laboratory test for assaying a genetic sample from the individual for the presence of at least one allele of a biomarker associated with ASD; wherein the presence in the genetic sample of the at least one allele of a biomarker associated with ASD indicates that the individual is affected with ASD or predisposed to ASD.

In one embodiment of the in vitro diagnostic test of the present invention, the at least one allele of the biomarker associated with ASD is a single nucleotide polymorphism comprising a single nucleotide polymorphism set forth in Tables 2, 3, 4, 5, 6, 7, or 8 or set forth as rs2277049, rs757099, rs7785107, rs7725785, rs2287581, rs1231339, rs2180055, rs11671930, rs7950390, rs12266938, rs3861787, rs1827924, rs17738966, rs317985, rs730168, rs10519124, rs6482516, or rs2297172, mutants, alleles or complementary sequences thereof, or any combination thereof, e.g., (a) rs2277049, rs757099, rs7785107, rs7725785, rs2287581, rs1231339, rs2180055, and rs11671930, which are all associated with the language impairment subtype; (b) rs7785107, rs7950390, rs12266938, and rs3861787, which are all associated with the intermediate subtype; (c) rs1827924, rs17738966, rs11671930, rs3861787, and rs317985 which are all associated with the moderate subtype, and (d) rs12266938, rs730168, rs10519124, rs6482516, rs11671930, rs2297172, rs317985, rs1827924, rs1231339, rs757099, and rs7725785, which are all associated with the mild subtype as set forth in Table 7.

In one embodiment of the in vitro diagnostic test of the present invention, the at least one laboratory test for assaying the presence of at least one allele of a biomarker associated with ASD comprises an array based assay such as a microarray.

In yet another aspect of the invention, a method is provided for diagnosing a patient as predisposed to having an autism spectrum disorder comprising identifying in a patient a biomarker comprising (a) preparing samples of control and experimental DNA, wherein the experimental DNA is generated from a nucleic acid sample isolated from a subject suspected of being afflicted with the at least one autism spectrum disorder and the control DNA is generated from a nucleic acid sample isolated from a healthy individual; (b) preparing one or more microarrays comprising a plurality of different oligonucleotides having specificity for at least one allele of the biomarker associated with ASD comprising a single nucleotide polymorphism set forth in Tables 2, 3, 4, 5, 6, 7, or 8 or set forth as rs2277049, rs757099, rs7785107, rs7725785, rs2287581, rs1231339, rs2180055, rs11671930, rs7950390, rs12266938, rs3861787, rs1827924, rs17738966, rs317985, rs730168, rs10519124, rs6482516, or rs2297172, or variants, mutants, alleles or complementary sequences thereof, or any combination thereof associated with the at least one autism spectrum disorder; (c) applying the prepared samples to the one or more microarrays to allow hybridization between the oligonucleotides and the control DNA and the oligonucleotide and the experimental DNA; (d) identifying the oligonucleotides on the microarray which display differential hybridization to the experimental DNA relative to the control DNA thereby identifying in a patient a biomarker or biomarker set profile for the at least one autism spectrum disorder.

In various embodiments of the diagnosing methods of the present invention, the autism spectrum disorder comprises autistic disorder, pervasive developmental disorder-not otherwise specified (PDD-NOS), including atypical autism, Asperger's Disorder, or a combination thereof.

Methods of Identifying or Characterizing Therapeutic Compounds

Another aspect of the invention is identification or screening of chemical or genetic compounds, derivatives thereof, and compositions including same that are effective in treatment of autism or autism spectrum disorders and individuals at risk thereof. The amount that is administered to an individual in need of therapy or prophylaxis, its formulation, and the timing and route of delivery is effective to reduce the number or severity of symptoms, to slow or limit progression of symptoms, to inhibit expression of one or more of the aforementioned biomarker associated differentially expressed genes in Table 1 or Table 7 that are transcribed at a higher level in autism or autism spectrum disorders, to activate expression of one or more of the aforementioned biomarker associated differentially expressed genes in Table 1 or Table 7 that are transcribed at a lower level in autism or autism spectrum disorders, or any combination thereof. Determination of such amounts, formulations, and timing and route of drug delivery is within the skill of persons conducting in vitro assays, in vivo studies of animal models, and human clinical trials.

Accordingly, in one aspect of the present invention, the biomarkers identified using the methods of the present invention are useful for the identification of new agents or drugs for the treatment of autism and autism spectrum disorders.

Thus, in one embodiment of the present invention, a method of identifying a candidate agent for treating autism or autism spectrum disorders is provided said method comprising: (a) contacting a biological sample from a patient with the candidate agent and determining the level of gene expression of one or more of the genes in Tables 1 or 7, associated with one or more of the biomarkers described herein; (b) determining the level of expression of one or more of the genes in a biological sample not contacted with the candidate agent; (c) observing the effect of the candidate agent by comparing the level of expression of the genes in the biological sample contacted with the candidate agent and the level of expression of the corresponding genes in the biological sample not contacted with the candidate agent; and (d) identifying the agent from the observed effect, wherein an at least 1%, 2%, 5%, 10% difference between the level of expression of the gene or combination of genes in the biological sample contacted with the candidate agent and the level of expression of the corresponding gene or combination of genes in the biological sample not contacted with the candidate agent is an indication of an effect of the candidate agent.

In one embodiment of the candidate agent identifying method, the biomarker is a biomarker for diagnostically distinguishing between autism spectrum disorders comprising at least one single nucleotide polymorphism set forth in Tables 2, 3, 4, 5, 6, 7, or 8 or set forth as rs2277049, rs757099, rs7785107, rs7725785, rs2287581, rs1231339, rs2180055, rs11671930, rs7950390, rs12266938, rs3861787, rs1827924, rs17738966, rs317985, rs730168, rs10519124, rs6482516, or rs2297172, or variants, mutants, alleles or complementary sequences thereof, or any combination thereof.

In another embodiment of the invention, a method of producing a drug comprising the steps of the candidate agent identifying method according to the invention (i) synthesizing the candidate agent identified in step (c) above or an analog or derivative thereof in an amount sufficient to provide said drug in a therapeutically effective amount to a subject; and/or (ii) combining the drug candidate the candidate agent identified in step (c) above or an analog or derivative thereof with a pharmaceutically acceptable carrier.

In one embodiment of the present invention a method is provided for identifying agents which alter those neurological functions and disorders associated with ASD pathophysiology comprising (a) providing cells expressing at least one allele of the biomarker associated with ASD comprising a single nucleotide polymorphism set forth in Tables 2, 3, 4, 5, 6, 7, or 8 or set forth as rs2277049, rs757099, rs7785107, rs7725785, rs2287581, rs1231339, rs2180055, rs11671930, rs7950390, rs12266938, rs3861787, rs1827924, rs17738966, rs317985, rs730168, rs10519124, rs6482516, or rs2297172, or variants, mutants, alleles or complementary sequences thereof, or any combination thereof associated with the at least one autism spectrum disorder; (b) providing cells which express the cognate wild type sequences corresponding to the single nucleotide polymorphism-containing nucleic acids; (c) contacting the cells from each sample with a test agent and analyzing whether said agent alters the neurological functions and disorders associated with ASD pathophysiology of step a) relative to those of step b), thereby identifying agents which alter neurological functions and disorders associated with autism pathophysiology.

In yet another embodiment of the present invention, the aforementioned method is used to identify those agents that alter those neurological functions and disorders associated with ASD pathophysiology comprising neuronal signaling and/or morphology, cell growth and death, embryogenesis, chromatin remodeling, myelination, oligodendrocyte differentiation, and complement activation, in addition to disorders that include demyelinating diseases, neuron dysfunction, nerve degeneration, and inflammation or cadherin-mediated cellular adhesion, or any combination thereof.

In yet another embodiment of the present invention, the aforementioned method is used to identify those agents that alter nervous system development, axon guidance, synaptic transmission or plasticity, long-term potentiation, neuron toxicity, Purkinje cell differentiation, cerebella development, embryonic development, regulation of actin networks, digestion, inflammation, oxidative stress, epilepsy, apoptosis, morphogenesis, cell survival, differentiation, the unfolded protein response, Type II diabetes and insulin signaling, digestion, liver toxicity (hepatic stellate cell activation, fibrosis, and cholestasis), endocrine function, circadian rhythm, cholesterol metabolism and the steroidogenesis pathway, or any combination thereof.

A screening method may comprise administering a candidate compound to an organism or incubating a candidate compound with a cell, and then determining whether or not gene expression of a gene associated with a biomarker as described herein as set forth in Table 1 or 7 is modulated. Such modulation may be an increase or decrease in activity that partially or fully compensates for a change that is associated with or may cause neurological disease. Differentially expressed gene expression may be increased at the level of rate of transcriptional initiation, rate of transcriptional elongation, stability of transcript, translation of transcript, rate of translational initiation, rate of translational elongation, stability of protein, rate of protein folding, proportion of protein in active conformation, functional efficiency of protein (e.g., activation or repression of transcription), or combinations thereof. See, for example, U.S. Pat. Nos. 5,071,773 and 5,262,300. High-throughput screening assays are possible (e.g., by using parallel processing and/or robotics).

The screening method may comprise incubating a candidate compound with a cell containing a reporter construct, the reporter construct comprising transcription regulatory region covalently linked in a cis configuration to a downstream gene encoding an assayable product; and measuring production of the assayable product. A candidate compound which increases production of the assayable product would be identified as an agent which activates gene or cDNA expression while a candidate compound which decreases production of the assayable product would be identified as an agent which inhibits gene or cDNA expression. See, for example, U.S. Pat. Nos. 5,849,493 and 5,863,733.

The screening method may comprise measuring in vitro transcription from a reporter construct in the presence or absence of a candidate compound (the reporter construct comprising a transcription regulatory region) and then determining whether transcription is altered by the presence of the candidate compound. In vitro transcription may be assayed using a cell-free extract, partially purified fractions of the cell, purified transcription factors or RNA polymerase, or combinations thereof. See, for example, U.S. Pat. Nos. 5,453,362, 5,534,410, 5,563,036, 5,637,686, 5,708,158 and 5,710,025.

Techniques for measuring transcriptional or translational activity in vivo are known in the art. For example, a nuclear run-on assay may be employed to measure transcription of a reporter gene. Translation of the reporter gene may be measured by determining the activity of the translation product. The activity of a reporter gene can be measured by determining one or more of transcription of polynucleotide product (e.g., RT-PCR of GFP transcripts), translation of polypeptide product (e.g., immunoassay of GFP protein), and enzymatic activity of the reporter protein per se (e.g., fluorescence of GFP or energy transfer thereof).

As used herein, differential expression may refer to a lower expression level or to a higher expression. In preferred embodiments, the difference in expression level is statistically significant for each of the differentially expressed genes in Tables 1 or 7, associated with one or more of the biomarkers described herein, on the set. In preferred embodiments, the difference in expression is at least 10%, 20%, 30%, 40%, 50%, 60%, 70%, 80%, 90%, 100%, 150%, 200%, 300%, 400%, or 500% greater in the experimental DNA than in the control DNA, or vice versa. In another preferred embodiment, the difference in expression is at least about 1.22-fold, 1.5-fold, 2-fold, 2.5-fold, 3-fold, 3.5-fold, 4-fold, 4.5-fold, 5-fold, 6-fold, 7-fold, 8-fold, 9-fold, 10-fold, 12-fold, 14-fold, 16-fold, 18-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold greater (or intermediate ranges thereof as another example) in the experimental DNA than in the control DNA, or vice versa. A gene profile may comprise all of the genes in Tables 1 or 7, associated with one or more of the biomarkers described herein which are differentially expressed between the control and experimental DNAs or it may comprise a subset of those genes. In some embodiments, the gene profile comprises at least 1%, 2%, 3%, 4%, 5%, 10%, 20%, 30%, 40%, 50%, 60%, 70%, 80%, 90%, 95%, 98%, 99% or 100% (or intermediate ranges thereof as another example) of the genes in Tables 1 or 7, associated with one or more of the biomarkers described herein having differential expression. Differentially expressed genes showing large, reproducible changes in expression between the two samples are preferred in some embodiments. In preferred embodiments, the differentially expressed gene profile further comprises a subset of values associated with the expression level of each of the differentially expressed gene in the profile, such that differentially expressed gene profile allows the identification of a biological and/or pathological condition, an agent and/or its biological mechanism of action, or a physiological process.

The preparation of samples of control and experimental DNA may be carried out using techniques known in the art. The DNA molecules analyzed by the present invention may be from any clinically relevant source. In one embodiment, the DNA is derived from RNA, including, but by no means limited to, total cellular RNA, poly(A)⁺ messenger RNA (mRNA) or fraction thereof, cytoplasmic mRNA, or RNA transcribed from cDNA (i.e., cRNA; see, e.g., U.S. Pat. Nos. 5,545,522, 5,891,636, or 5,716,785). Methods for preparing total and poly(A)⁺ RNA are well known in the art, and are described generally, e.g., in Sambrook et al., MOLECULAR CLONING—A LABORATORY MANUAL (2ND ED.), Vols. 1-3, Cold Spring Harbor Laboratory, Cold Spring Harbor, N.Y. (1989). In one embodiment, RNA is extracted from a sample of cells of the various tissue types of interest, such as the lymphoblastoid cell or lymphoblastoid cell line derived therefrom or from the aforementioned neuronal tissue types, using guanidinium thiocyanate lysis followed by CsCl centrifugation (Chirgwin et al., 1979, Biochemistry 18:5294-5299). In another embodiment, total RNA is extracted using a silica gel-based column, commercially available examples of which include RNeasy (Qiagen, Valencia, Calif.) and StrataPrep (Stratagene, La Jolla, Calif.). Poly(A)⁺ RNA can be selected, e.g., by selection with oligo-dT cellulose or, alternatively, by oligo-dT primed reverse transcription of total cellular RNA. In one embodiment, RNA can be fragmented by methods known in the art, e.g., by incubation with ZnCl₂, to generate fragments of RNA. In another embodiment, the polynucleotide molecules analyzed by the invention comprise PCR products of amplified polynucleotides (e.g. RNA or cDNA, among others). DNA molecules that are poorly expressed in particular cells may be enriched using normalization techniques (Bonaldo et al., 1996, Genome Res. 6:791-806).

The DNAs may be detectably labeled at one or more nucleotides. Any method known in the art may be used to detectably label the DNAs. Preferably, this labeling incorporates the label uniformly along the length of the RNA, and more preferably, the labeling is carried out at a high degree of efficiency. One embodiment for this labeling uses oligo-dT primed reverse transcription to incorporate the label; however, conventional methods of this method are biased toward generating 3′ end fragments. Thus, in one embodiment, random primers (e.g., 9-mers) are used in reverse transcription to uniformly incorporate labeled nucleotides over the full length of the DNAs. Alternatively, random primers may be used in conjunction with PCR methods or T7 promoter-based in vitro transcription methods in order to amplify the cDNAs.

In one embodiment, the detectable label is a luminescent label. For example, fluorescent labels, bioluminescent labels, chemiluminescent labels, and colorimetric labels may be used in the present invention. In one preferred embodiment, the label is a fluorescent label, such as a fluorescein, a phosphor, a rhodamine, or a polymethine dye derivative. Examples of commercially available fluorescent labels include, for example, fluorescent phosphoramidites such as FluorePrime (Amersham Pharmacia, Piscataway, N.J.), Fluoredite (Millipore, Bedford, Mass.), FAM (ABI, Foster City, Calif.), and Cy3 or Cy5 (Amersham Pharmacia, Piscataway, N.J.). In another embodiment, the detectable label is a radiolabeled nucleotide.

In a further preferred embodiment, the experimental DNAs are labeled differentially from the control DNA, especially if both the DNA types are hybridized to the same microarray. The control DNA can comprise target polynucleotide molecules from normal individuals (i.e., those not afflicted with the neurological disorder or subjects who have not undergone to therapeutic treatment). In one preferred embodiment, the control DNA comprises target polynucleotide molecules pooled from samples from normal individuals. In one embodiment of the methods for generating a gene profile of a therapeutic treatment, the control DNA is derived from the same subject, but taken at a different time point, such as before, during or after the therapeutic treatment.

Nucleic acid hybridization and wash conditions are chosen so that the DNA molecules specifically bind or specifically hybridize to the complementary polynucleotide sequences of the array, preferably to a specific array site, wherein its complementary DNA is located. Arrays containing double-stranded probe DNA situated thereon are preferably subjected to denaturing conditions to render the DNA single-stranded prior to contacting with the DNA molecules. Arrays containing single-stranded probe DNA (e.g., synthetic oligodeoxyribonucleic acids) may need to be denatured prior to contacting with the DNA molecules. Optimal hybridization conditions will depend on the length (e.g., oligomer versus polynucleotide greater than 200 bases) and type (e.g., RNA, or DNA) of probe and target nucleic acids. One of skill in the art will appreciate that as the oligonucleotides become shorter, it may become necessary to adjust their length to achieve a relatively uniform melting temperature for satisfactory hybridization results. General parameters for specific (i.e., stringent) hybridization conditions for nucleic acids are described in Sambrook et al., MOLECULAR CLONING—A LABORATORY MANUAL (2ND ED.), Vols. 1-3, Cold Spring Harbor Laboratory, Cold Spring Harbor, N.Y. (1989), and in Ausubel et al., CURRENT PROTOCOLS IN MOLECULAR BIOLOGY, vol. 2, Current Protocols Publishing, New York (1994). Typical hybridization conditions for the cDNA microarrays of Schena et al. are hybridization in 5×SSC plus 0.2% SDS at 65° C. for four hours, followed by washes at 25° C. in low stringency wash buffer (1×SSC plus 0.2% SDS), followed by 10 minutes at 25° C. in higher stringency wash buffer (0.1×SSC plus 0.2% SDS) (Schena et al., Proc. Natl. Acad. Sci. U.S.A. 93:10614 (1993)). Useful hybridization conditions are also provided in, e.g., Tijessen, 1993, HYBRIDIZATION WITH NUCLEIC ACID PROBES, Elsevier Science Publishers B. V.; and Kricka, 1992, NONISOTOPIC DNA PROBE TECHNIQUES, Academic Press, San Diego, Calif. Hybridization conditions may include hybridization at a temperature at or near the mean melting temperature of the probes (e.g., within 5° C., more preferably within 2° C.) in 1 M NaCl, 50 mM MES buffer (pH 6.5), 0.5% sodium sarcosine and 30% formamide.

When fluorescently labeled DNAs are used in the aforementioned methods, the fluorescence emissions at each site of a microarray may be, preferably, detected by scanning confocal laser microscopy. In one embodiment, a separate scan, using the appropriate excitation line, is carried out for each of the two fluorophores used. Alternatively, a laser may be used that allows simultaneous specimen illumination at wavelengths specific to the two fluorophores and emissions from the two fluorophores can be analyzed simultaneously (see Shalon et al., 1996, “A DNA microarray system for analyzing complex DNA samples using two-color fluorescent probe hybridization,” Genome Research 6:639-645, which is incorporated by reference in its entirety for all purposes). In one preferred embodiment, the arrays are scanned with a laser fluorescent scanner with a computer controlled X-Y stage and a microscope objective. Sequential excitation of the two fluorophores is achieved with a multi-line, mixed gas laser and the emitted light is split by wavelength and detected with two photomultiplier tubes. Fluorescence laser scanning devices are described in Schena et al., Genome Res. 6:639-645 (1996), and in other references cited herein. Alternatively, the fiber-optic bundle described by Ferguson et al., Nature Biotech. 14:1681-1684 (1996), may be used to monitor differentially expressed gene or DNA abundance levels at a large number of sites simultaneously.

Signals may be recorded and, in a preferred embodiment, analyzed by computer, e.g., using a 12 or 16 bit analog to digital board. In one embodiment the scanned image is despeckled using a graphics program (e.g., Hijaak Graphics Suite) and then analyzed using an image gridding program that creates a spreadsheet of the average hybridization at each wavelength at each site. If necessary, an experimentally determined correction for “cross talk” (or overlap) between the channels for the two fluors may be made. For any particular hybridization site on the transcript array, a ratio of the emission of the two fluorophores can be calculated. The ratio is independent of the absolute expression level of the differentially expressed gene, but is useful for differentially expressed genes whose expression is significantly modulated in association with the different neurological conditions.

In another embodiment of the present invention, changes in differentially expressed gene expression may be assayed in at least one cell of a subject by measuring transcriptional initiation, transcript stability, translation of transcript into protein product, protein stability, or a combination thereof. The gene, gene products, transcript, or polypeptide can be assayed by techniques such as in vitro transcription, quantitative nuclease protection assay (qNPA) analysis, focused gene chip analysis, Northern hybridization, nucleic acid hybridization, reverse transcription-polymerase chain reaction (RT-PCR), run-on transcription, Southern hybridization, electrophoretic mobility shift assay (EMSA), fluorescent or histochemical staining, microscopy and digital image analysis, and fluorescence activated cell analysis or sorting (FACS).

A reporter or selectable marker gene whose protein product is easily assayed may be used for convenient detection. Reporter genes include, for example, alkaline phosphatase, β-galactosidase (LacZ), chloramphenicol acetyltransferase (CAT), β-glucoronidase (GUS), bacterial/insect/marine invertebrate luciferases (LUC), green and red fluorescent proteins (GFP and RFP, respectively), horseradish peroxidase (HRP), β-lactamase, and derivatives thereof (e.g., blue EBFP, cyan ECFP, yellow-green EYFP, destabilized GFP variants, stabilized GFP variants, or fusion variants sold as LIVING COLORS fluorescent proteins by Clontech). Reporter genes would use cognate substrates that are preferably assayed by a chromogen, fluorescent, or luminescent signal. Alternatively, assay product may be tagged with a heterologous epitope (e.g., FLAG, MYC, SV40 T antigen, glutathione transferase, hexahistidine, maltose binding protein) for which cognate antibodies or affinity resins are available.

In yet another aspect of the present invention, the biomarkers identified using the methods of the present invention are useful for testing the efficacy of compounds in the treatment of autism and autism spectrum disorders.

In yet another aspect, the present invention also provides a method of identifying an effective treatment regimen for a subject with an autism spectrum disorder, comprising detecting one or more biomarkers described in embodiments of the invention and correlated with an effective treatment regimen for an autism spectrum disorder.

In another embodiment, the present invention provides a method of identifying an effective treatment regimen for a subject with an autism spectrum disorder, comprising: a) correlating the presence of one or more biomarkers in a test subject with an autism spectrum disorder for whom an effective treatment regimen has been identified; and b) detecting the one or more markers of step (a) in the subject, thereby identifying an effective treatment regimen for the subject. Subjects who respond well to particular treatment protocols can thus be analyzed for specific biomarkers and a correlation can be established according to the methods provided herein. Alternatively, subjects who respond poorly to a particular treatment regimen can also be analyzed for particular biomarkers correlated with the poor response. Then, a subject who is a candidate for treatment for an autism spectrum disorder can be assessed for the presence of the appropriate biomarkers and the most appropriate treatment regimen can be provided.

In yet another embodiment of the effective treatment regimen method of the present invention, the subject undergoes a selected physiological change as a result of treatment, wherein the selected physiological change includes one or more improvements in social interaction, language abilities, restricted interests, repetitive behaviors, sleep disorders, seizures, gastrointestinal, hepatic, and mitochondrial function, neural inflammation, or a combination thereof.

In various embodiments of the effective treatment regimen method of the present invention, the autism spectrum disorder comprises autistic disorder, pervasive developmental disorder-not otherwise specified (PDD-NOS), including atypical autism, Asperger's Disorder, or a combination thereof.

In yet another aspect of the invention provides methods of identifying, or predicting the efficacy of, test compounds. In particular, the invention provides methods of identifying compounds which mimic the effects of behavioral therapies. In still another aspect, the systems and methods described herein provide a method for predicting efficacy of a test compound for altering a behavioral response, by obtaining a database, treating a test animal or human (e.g., a control animal or human that has not undergone other therapies, such as behavioral therapy) with the test compound, and comparing genomic or cDNA expression data of tissue samples from the animal or human treated with the test compound to measure a degree of similarity with one or more differentially expressed gene profiles of the genes in Tables 1 or 7, associated with one or more of the biomarkers in said database. In certain embodiments, the untreated animal or human exhibits a psychological and/or behavioral abnormality possessed by the animals or humans used to generate the database prior to administration of the behavioral therapy.

Thus, in yet another embodiment of the present invention, a method is provided for predicting efficacy of a test compound for altering a behavioral response in a subject with at least one autism spectrum disorder comprising: (a) preparing a microarray comprising a plurality of different oligonucleotides, wherein the oligonucleotides have specificity for at least one allele of the biomarker associated with ASD comprising a single nucleotide polymorphism set forth in Tables 2, 3, 4, 5, 6, 7, or 8 or set forth as rs2277049, rs757099, rs7785107, rs7725785, rs2287581, rs1231339, rs2180055, rs11671930, rs7950390, rs12266938, rs3861787, rs1827924, rs17738966, rs317985, rs730168, rs10519124, rs6482516, or rs2297172, or variants, mutants, alleles or complementary sequences thereof, or any combination thereof associated with at least one autism spectrum disorder; (b) obtaining a differential biomarker profile representative of the biomarker profile of at least one sample of a selected tissue type from a subject subjected to each of at least one of a plurality of selected behavioral therapies which promote the behavioral response; (c) administering the test compound to the subject; and (d) comparing a differential biomarker profile data in at least one sample of the selected tissue type from the subject treated with the test compound to determine a degree of similarity with one or more differential biomarker profile associated with an autism spectrum disorder; wherein the predicted efficacy of the test compound for altering the behavioral response is correlated to said degree of similarity.

In yet another embodiment of the compound efficacy testing method of the present invention, the behavioral therapy comprises applied behavior analysis (ABA) intervention methods, dietary changes, exercise, massage therapy, group therapy, talk therapy, play therapy, conditioning, or alternative therapies such as sensory integration and auditory integration therapies.

In various embodiments of the compound efficacy predicting method of the present invention, the autism spectrum disorder comprises autistic disorder, pervasive developmental disorder-not otherwise specified (PDD-NOS), including atypical autism, Asperger's Disorder, or a combination thereof.

From such a database, biological targets for intervention can be identified, such as potential therapeutics (e.g., genes or cDNAs that are upregulated and thus may exert a beneficial effect on the physiology and/or behavior of the subject), potential receptor targets (e.g., receptors associated with upregulated proteins, the activation of which receptors may exert a beneficial effect on the physiology and/or behavior of the subject; or receptors associated with downregulated proteins, the inhibition of which may exert a beneficial effect on the physiology and/or behavior of the subject). In certain embodiments, one or more genes or one or more cDNAs, the expression of which differs by a statistically significant amount in a treated subject as compared to an untreated control, may be selected as targets for intervention.

In one embodiment of the foregoing methods, the test subject or animal is a human. In another embodiment, the animal is a non-human animal. Such non-human animals include vertebrates such as rodents, non-human primates, ovines, bovines, ruminants, lagomorphs, porcines, caprines, equines, canines, felines, ayes, etc. Preferred non-human animals are selected from the order Rodentia, most preferably mice. The term “order Rodentia” refers to rodents (i.e., placental mammals (Class Euthria) which include the family Muridae (rats and mice)). In a specific embodiment, the test animal is a mammal, a primate, a rodent, a mouse, a rat, a guinea pig, a rabbit or a human.

In some embodiments of the methods described herein, the test compound comprises an antibody or fragment thereof, nucleic acid molecules, peptides, polypeptides, peptidomimetics, RNAi constructs, antisense reagent, oligonucleotides, ribozymes, a small molecule drug, or a nutritional or herbal supplement, or a combination thereof. Test compounds can be screened individually, in combination with one or more other compounds, or as a library of compounds.

In general, test compounds for modulation of neurological disorders, including those autism spectrum disorders such as autistic disorder, pervasive developmental disorder-not otherwise specified (PDD-NOS), including atypical autism, Asperger's Disorder, or a combination thereof, can be identified from large libraries of natural products or synthetic (or semi-synthetic) extracts or chemical libraries according to methods known in the art. Those skilled in the field of drug discovery and development will understand that the precise source of test extracts or compounds is not critical to the screening procedure(s) of the invention. Accordingly, virtually any number of chemical extracts or compounds can be screened using the exemplary methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available, e.g., Chembridge (San Diego, Calif.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are generated, if desired, according to methods known in the art, e.g., by standard extraction and fractionation methods. Furthermore, if desired, any library or compound is readily modified using standard chemical, physical, or biochemical methods.

In another embodiment of the invention, a pharmaceutical preparation comprising a compound according to the invention is provided.

Small molecule test agents may then be screened in any of a number of assays to identify those with potential therapeutic applications. The term “small molecule” refers to a compound having a molecular weight less than about 2500 amu, preferably less than about 2000 amu, even more preferably less than about 1500 amu, still more preferably less than about 1000 amu, or most preferably less than about 750 amu. For example, subjects or tissue samples may be treated with such test agents to identify those that produce similar changes in expression of the targets, or produce similar gene profiles, as can be obtained by administration of behavioral therapy. Alternatively or additionally, such test agents may be screened against one or more target receptors to identify compounds that agonize or antagonize these receptors, singly or in combination, e.g., so as to reproduce or mimic the effect of behavioral therapy.

Compounds that induce a desired effect on targets, tissue, or subjects may then be selected for clinical development, and may be subjected to further testing, e.g., therapeutic profiling, such as testing for efficacy and toxicity in subjects. Analogs of selected compounds, e.g., compounds having similar cores but varying substituents and stereochemistry, may similarly be developed and tested. Agents that have acceptable characteristics for therapeutic use in humans or animals may be prepared as pharmaceutical preparations, e.g., with a pharmaceutically acceptable excipient (such as a non-pyrogenic or sterile excipient). Such agents may also be licensed to a manufacturer for development and/or commercialization, e.g., for manufacture and sale of a pharmaceutical preparation comprising said selected agent.

The test compound may be administered to the subject or animal using any mode of administration, including, intravenous, subcutaneous, intramuscular, intrastemal, topical, liposome-mediate, rectal, intravaginal, opthalmic, intracranial, intraspinal or intraorbital. The test compound may be administered once or more than once as part of a treatment regimen. In some embodiments, additional test compounds or agents may be administered to the subject animal to ascertain the efficacy of the test compound or the combination of test compounds or agents. In some embodiments, a differentially expressed gene profile may also be obtained from the subject or animal prior to treatment with the test agent.

In one embodiment of the methods for determining a biomarker profile for the administration of a therapeutic treatment, administration of therapeutic treatment results in a physiological change in the subject, such as a beneficial change. In a specific embodiment, the physiological change comprises one or more improvements in social interaction, language abilities, restricted interests, repetitive behaviors, sleep disorders, seizures, gastrointestinal, hepatic, and mitochondrial function, neural inflammation, or a combination thereof. In another embodiment, control DNA may be derived from the subject(s) prior to administration of the therapeutic treatment, or from a subject or group of subjects who do not receive the therapeutic treatment.

In yet another embodiment of the method of the present invention, prior to administration of behavioral therapy, the subject shows at least one symptom of a psychological or physiological abnormality.

In yet another embodiment of the compound efficacy testing method of the present invention, step (a) comprises obtaining a differential biomarker profile representative of the differential biomarker profile of at least two samples of a selected tissue type.

In yet another embodiment of the compound efficacy testing method of the present invention, the selected tissue type comprises a neuronal tissue type.

In yet another embodiment of the compound efficacy testing method of the present invention, the neuronal tissue type is selected from the group consisting of olfactory bulb cells, cerebrospinal fluid, hypothalamus, amygdala, pituitary, nervous system, brainstem, cerebellum, cortex, frontal cortex, hippocampus, striatum, and thalamus.

In yet another embodiment of the compound efficacy testing method of the present invention, the selected tissue type is selected from the group consisting of lymphocytes, blood, or mucosal epithelial cells, brain, spinal cord, heart, arteries, esophagus, stomach, small intestine, large intestine, liver, pancreas, lungs, kidney, urinary tract, ovaries, breasts, uterus, testis, penis, colon, prostate, bone, muscle, cartilage, thyroid gland, adrenal gland, pituitary, bone marrow, blood, thymus, spleen, lymph nodes, skin, eye, ear, nose, teeth or tongue.

In yet another embodiment of the method of the present invention, the neuronal tissue type is selected from the group consisting of olfactory bulb cells, cerebrospinal fluid, hypothalamus, amygdala, pituitary, nervous system, brainstem, cerebellum, cortex, frontal cortex, hippocampus, striatum, and thalamus.

Kits

In yet another aspect of the invention, kits are provided for use in the methods described herein for diagnosing, or screening an individual for the risk of having or developing an autism spectrum disorder, or identifying candidate agent useful in treating an autism spectrum disorder comprising i) one or more of the autism spectrum disorders single nucleotide polymorphism biomarkers set forth in either Table 2, Table 3, Table 4, Table 5, Table 6, Table 7, Table 8, or variants, mutants, alleles or complementary sequences thereof, or any combination thereof that are associated with at least one autism spectrum disorder, and ii) instructions for use thereof.

In yet another aspect of the invention, a computer-readable medium on which is encoded programming code for analyzing and/or distinguishing between autism spectrum disorders from a plurality of data points wherein the computer-readable medium comprises single nucleotide polymorphism biomarkers for diagnosing autism and autism spectrum disorders comprising at least one single nucleotide polymorphism set forth as: rs2277049, rs757099, rs7785107, rs7725785, rs2287581, rs1231339, rs2180055, rs11671930, rs7950390, rs12266938, rs3861787, rs1827924, rs17738966, rs317985, rs730168, rs10519124, rs6482516, or rs2297172, or variants, mutants, alleles or complementary sequences thereof, or any combination thereof.

In some embodiments, the methods of correlating biomarkers with diagnosing and/or treatment regimens can be carried out using a computer database.

Thus in one embodiment, the present invention provides a computer-assisted method of identifying a proposed treatment for autism spectrum disorder comprising the steps of (a) storing a database of biological data for a plurality of patients, the biological data that is being stored including for each of said plurality of patients (i) a treatment type, (ii) at least one biomarker associated with autism spectrum disorder wherein the at least one biomarker comprises rs2277049, rs757099, rs7785107, rs7725785, rs2287581, rs1231339, rs2180055, rs11671930, rs7950390, rs12266938, rs3861787, rs1827924, rs17738966, rs317985, rs730168, rs10519124, rs6482516, or rs2297172, or variants, mutants, alleles or complementary sequences thereof, or any combination thereof and (iii) at least one disease progression measure for autism spectrum disorder from which treatment efficacy can be determined; and then (b) querying the database to determine the dependence on said biomarker of the effectiveness of a treatment type in treating autism spectrum disorder, to thereby identify a proposed treatment as an effective treatment for a subject carrying a biomarker correlated with autism spectrum disorder.

In yet another embodiment of the invention, in each of the screening methods, SNP biomarker profiling methods, drug discovery methods, compound efficacy testing methods, computer program for determining a biomarker profile, and kits specifically provided for supra (and infra) may also be, without any limitation, made and/or practiced with from at least one to at least 164, or any integer value thereof, different single nucleotide polymorphism biomarkers set forth in either Table 2, Table 3, Table 4, Table 5, Table 6, Table 7, Table 8, or variants, mutants, alleles or complementary sequences thereof, or any combination thereof.

Methods of Conducting Drug Discovery

Another aspect of the invention provides methods for conducting drug discovery related to the methods and autism and autism spectrum disorder biomarkers provided herein.

One aspect of the invention provides a method for conducting drug discovery comprising: (a) generating a database of differentially expressed gene profile data representative of the genetic expression response of at least one selected tissue type (for example, one of the aforementioned neuronal tissue types) from a subject or an animal that was subjected to at least one of a plurality of behavioral therapies and that has undergone a selected physiological change since commencement of the behavioral therapy; (b) selecting at least one differentially expressed gene profile from Table 1 or Table 7, which are associated with at least one biomarker associated with autism spectrum disorder wherein the at least one biomarker comprises rs2277049, rs757099, rs7785107, rs7725785, rs2287581, rs1231339, rs2180055, rs11671930, rs7950390, rs12266938, rs3861787, rs1827924, rs17738966, rs317985, rs730168, rs10519124, rs6482516, or rs2297172, or variants, mutants, alleles or complementary sequences thereof, or any combination thereof, and selecting at least one target as a function of the selected differentially expressed gene profiles; (c) screening a plurality of test agents in assays to obtain differentially expressed gene profile data associated with administration of the agents and comparing the obtained data with the one or more selected differentially expressed gene profiles; (d) selecting for clinical development test agents that exhibit a desired effect on the target as evidenced by the differentially expressed gene profiles data; (e) for test agents selected for clinical development, conducting therapeutic profiling of the test compound, or analogs thereof, for efficacy and toxicity in subjects or animals; and (f) selecting at least one test agent that has an acceptable therapeutic and/or toxicity profile.

Another aspect of the invention provides a method for conducting drug discovery comprising: (a) generating a database of differentially expressed gene profile data representative of the genetic expression response of at least one selected neuronal tissue type from a subject or an animal that was subjected to at least one of a plurality of behavioral therapies and that has undergone a selected physiological change since commencement of the behavioral therapy; (b) administering test agents to test subjects or animals to obtain differentially expressed gene profile data associated with administration of the agents and comparing the obtained data with the one or more selected differentially expressed gene profiles; (c) selecting test agents that induce profiles similar to profiles obtainable by administration of behavioral therapy; (d) conducting therapeutic profiling of the selected test compound(s), or analogs thereof, for efficacy and toxicity in subjects or animals; and (e) identifying a pharmaceutical preparation including one or more agents identified in step (e) as having an acceptable therapeutic and/or toxicity profile.

In one embodiment, the database of differentially expressed gene profile data representative of the genetic expression response of at least one selected neuronal tissue type from a subject or an animal that was subjected to at least one of a plurality of behavioral therapies and that has undergone a selected physiological change since commencement of the behavioral therapy comprises at least one differentially expressed gene profile from Table 1 or Table 7 which are associated with at least one biomarker associated with autism spectrum disorder wherein the at least one biomarker comprises rs2277049, rs757099, rs7785107, rs7725785, rs2287581, rs1231339, rs2180055, rs11671930, rs7950390, rs12266938, rs3861787, rs1827924, rs17738966, rs317985, rs730168, rs10519124, rs6482516, or rs2297172, or variants, mutants, alleles or complementary sequences thereof, or any combination thereof.

### EXAMPLES

The invention now being generally described, it will be more readily understood by reference to the following examples, which are included merely for purposes of illustration of certain aspects and embodiments of the present invention, and are not intended to limit the invention, as one skilled in the art would recognize from the teachings hereinabove and the following examples, that other DNA microarrays, neurological conditions, cognitive therapies or data analysis methods, all without limitation, can be employed, without departing from the scope of the invention as claimed. The contents of any patents, patent applications, patent publications, or scientific articles referenced anywhere in this application are herein incorporated in their entirety.

### Example 1

Reanalysis of published genome-wide association data from the Autism Genetics Resource Exchange (AGRE): The use of quantitative traits and subphenotypes for association analyses reveals novel autism subtype-dependent genetic polymorphisms

In this Example and Examples 2-7 infra, results are presented from a reanalysis of published genome-wide association data from the Autism Genetics Resource Exchange (AGRE) which employs the use of quantitative traits and subphenotypes for association analyses and reveals novel autism subtype-dependent genetic polymorphisms

The heterogeneity of symptoms associated with autism spectrum disorders (ASD) has presented a significant challenge to genetic analyses. Even when associations with genetic variants have been identified, it has been difficult to associate them with a specific trait or characteristic of autism. In Examples 2-7, quantitative trait analyses of ASD symptoms combined with case-control association analyses using distinct ASD subphenotypes identified on the basis of symptomatic profiles results in the identification of 18 statistically significant novel associations with single nucleotide polymorphisms (SNPs). The symptom categories included deficits in language usage, non-verbal communication, social development, and play skills, as well as insistence on sameness or ritualistic behaviors. Ten of the trait-associated SNPs, or quantitative trait loci (QTL), were associated with more than one subtype, providing replication of the identified QTL. Several of the QTL reside within rare copy number variants that have been previously reported in autistic samples. Pathway analyses of the genes associated with the QTL identified in this study implicate neurological functions and disorders associated with autism pathophysiology. This study underscores the advantage of incorporating both quantitative traits as well as subphenotypes into large-scale genome-wide analyses of complex disorders.

### Example 2

### GWA and ADI-R Data Used for this Study

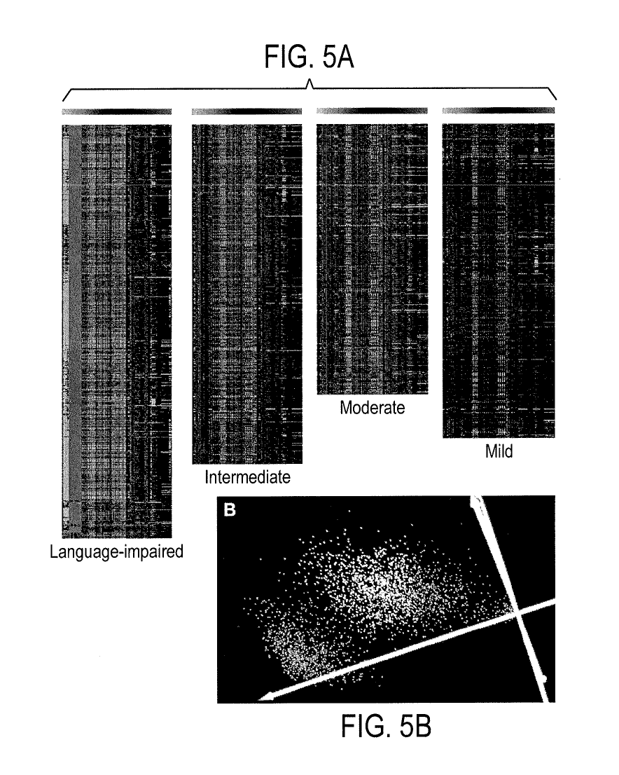

Genome-wide association data from the study by Wang et al. (9) was downloaded from the Autism Speaks website at ftp://ftp.autismspeaks.org/Data/CHOP_PLINK/AGRERELEASE.ped. For this study, the file named CHOP.clean100121 was used where the data was “cleaned” by Jennifer K. Lowe in the laboratory of Daniel H. Geschwind, M.D., Ph.D. at UCLA. The cleaning procedure involved extensive sample and pedigree validation, exclusion of SNPs a) missing >10% data, b) with HWE p<0.001, c) with MAF<0.01, and d) with >10 Mendelian errors. The final dataset included 4327 genotyped individuals and 513,312 SNPs on the Illumina HumanHap550 Bead Chip. Autism Diagnostic Interview-Revised (ADI-R) assessments for 2939 individuals were obtained from Autism Speaks through Dr. Vlad Kustanovich of the Autism Genetics Resource Exchange. Of these, 1867 individuals were among the cases genotyped by Wang et al. (9). Scores of 123 items on the ADI-R score sheets of each individual were analyzed as described by Hu and Steinberg (13) to identify ASD subtypes (that is, phenotypic subgroups) which are represented in FIG. 5.

### Example 3

### Determination of Quantitative Traits

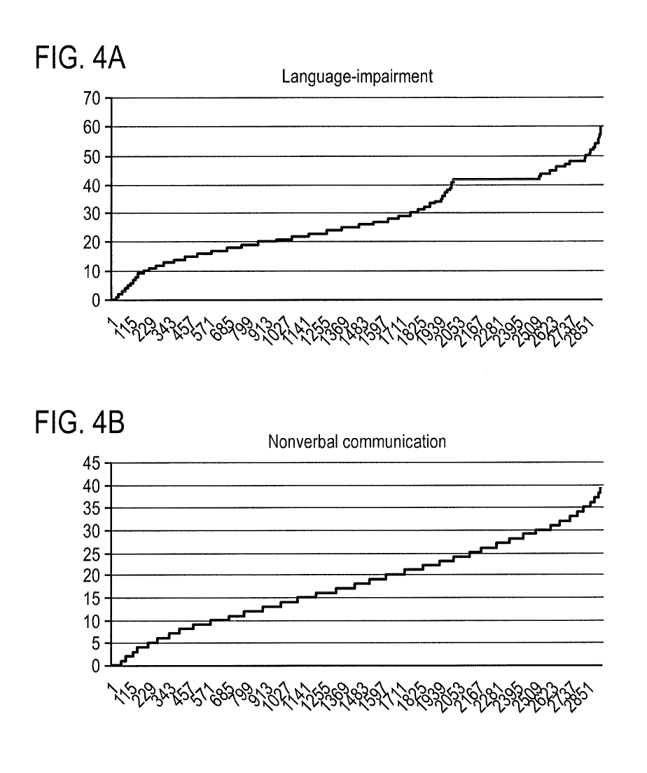

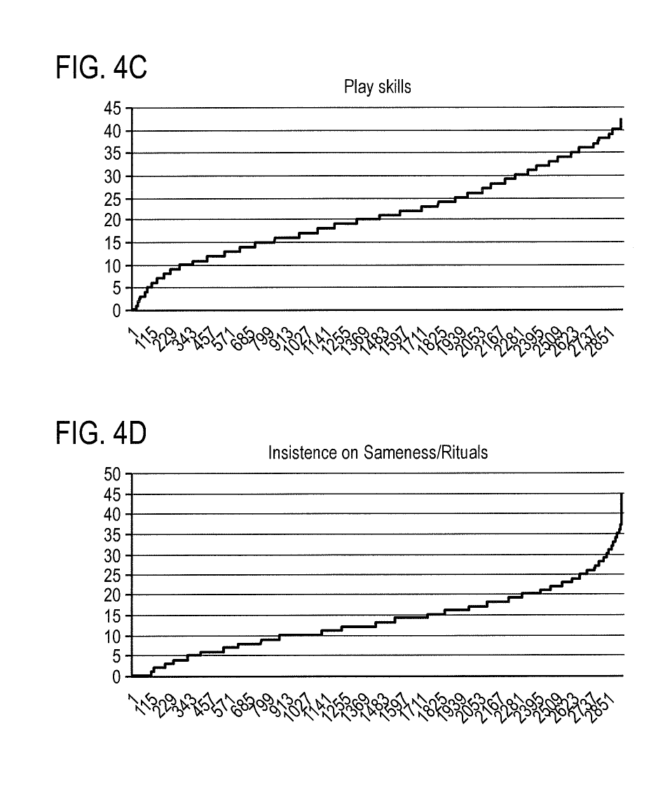

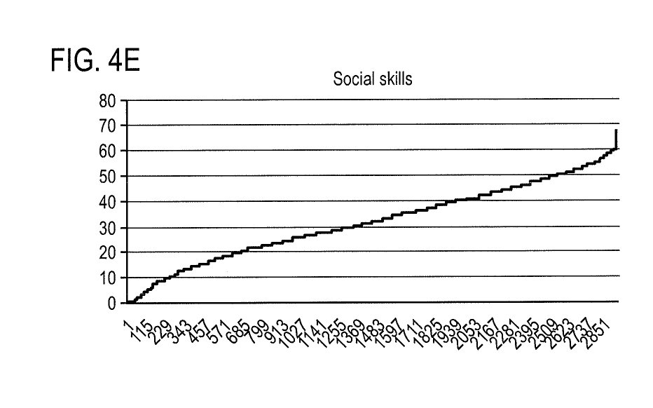

Raw item scores from the Autism Diagnostic Interview-Revised (ADI-R) score sheets of 2939 ASD cases were summed for 5 categories of symptoms, or traits, associated with ASD spoken language skills, non-verbal communication, play skills, social development, and insistence on sameness/ritualistic behaviors. The specific items used to obtain the total score per category for each individual, shown in Table 9, were noted in an earlier study (13) to exhibit average differences in severity among several subtypes of ASD, described below. The sum of items within each of the 5 categories were used as quantitative traits for genetic association analyses using the genotype data reported by Wang et al. (9). Profiles of the traits across the 2939 individuals are shown in FIG. 4.

### Example 4

### Subtyping of Individuals with ASD

Two thousand nine hundred and thirty nine (2939) individuals with ASD were divided into phenotypic subgroups using clustering tools within the MeV software package (Saeed et al. (2003) TM4: A free, open source system for microarray data analysis. BioTechniques 34,374-378.), as previously described by Hu and Steinberg (13). Briefly, subtyping of ASD individuals involved K-means cluster analysis (with K=4) of scores from 123 items from the ADI-R score sheets of each individual which were adjusted as described to fall into a severity range of 0 (normal) to 3 (highest severity). A figure of merit analysis (not shown) indicated that the individuals with ASD were optimally represented by 4 subgroups. A principal components analysis (PCA) was then used to verify that the 4 subgroups of individuals identified by the K-means cluster analyses were distinguishable by this unsupervised test. FIG. 5 shows the symptomatic profiles of the 4 ASD subtypes as well as their separation into discernible clusters by PCA. The subtypes are named “Language-impaired”, “Intermediate”, “Moderate”, and “Mild” and contain 639, 478, 363, and 387 cases, respectively.

### Example 5

### Quantitative Trait Association Analyses

Using the score totals for the 5 categories of autistic symptoms exhibited by each of the 1867 cases (that were genotyped by Wang (9)) as quantitative traits, PLINK (32), which is a program to analyze whole genome association data, was utilized to perform quantitative trait association analyses with the genotype data reported by Wang et al. (9). This analysis identified sets of SNPs that were associated with each of the 5 categories of autistic symptoms. Based on the results of each of the 5 analyses (Table 1), SNPs were selected with unadjusted p-values <10⁻⁵, which prioritized SNPs filtered by association with quantitative traits relevant to ASD. These filtered sets of SNPs (167 in total) were used in case-control association analyses, described below.

### Example 6

### Case-Control Association Tests

For each set of the filtered SNPs associated with each symptom category, i.e., the quantitative trait loci (QTL), additional association analyses using PLINK were performed between the 2438 control and each of the 4 subtypes as well as the combined group of 1867 cases. It should be noted that each ASD subgroup represents an entirely separate cohort of cases. From each of the 5 sets of genetic association analyses with subtypes and QTL (Tables 2-6), we selected SNPs associated with each ASD subtype with a Bonferroni-adjusted p-value S 0.05 and combined them (a total of 18 unique SNPs) for a second case-control association analysis using the combined and subtyped ASD cases against controls. The results of this final analysis are shown in Table 7.

Pathway Analysis.

Pathway Studio 7 software (Ariadne Genomics, Inc.) was used to generate relational gene networks using the SNP-containing genes listed in Table 7.

### Example 7

The overarching goal of these studies was to identify single nucleotide polymorphisms (SNPs) that are both associated with autistic traits and with clinical subtypes of autism. To accomplish this, quantitative trait analysis and ASD subtype association analyses were combined using the wealth of genome-wide association (GWAS) data published by the AGRE consortium of autism researchers in 2009 (9).

Quantitative Trait Association Analyses

The flowchart in FIG. 1 describes the analyses that were used to derive the final set of 18 novel and statistically significant SNPs that associate with subtypes of ASD. Raw item scores from the ADI-R score sheets of 2939 ASD cases were summed for spoken language skills, non-verbal communication, play skills, social development, and insistence on sameness/rituals, as described previously (13). The specific items used to obtain the total score per “trait” category for each individual are shown in Table 9 and the profiles of total scores for each category are shown for the 2939 individuals in FIG. 4. Quantitative trait association analyses was then conducted using the distribution of scores in each of the categories to identify sets of SNPs (nominal p≦10⁻⁵) that associate with symptomatic severity of each of the behaviors listed in Table 9. These sets of symptom-associated SNPs (or quantitative trait loci, QTL) are shown in Table 1.

ASD Subtype-Dependent Genetic Association Analyses with Trait-Associated SNPs

Next, cluster analyses was performed as described by Hu and Steinberg (13) to divide the autistic cases into 4 phenotypic subgroups according to symptomatic severity profiles derived from 123 items on the ADI-R assessments. This subtyping procedure, reduces the behavioral/symptomatic heterogeneity among the cases within each subgroup, and restricts the genetic heterogeneity within each subgroup. The results of K-means cluster analyses (K=4) of the ADIR data from the 2939 individuals (which included the 1867 genotyped cases from the GWA study (9)) are shown in FIG. 5. The resulting phenotypic subgroups were then used in genetic association analyses with the 167 filtered SNPs derived from the quantitative trait association analyses supra, where the 1867 cases were either divided into 4 ASD subtypes or used as a combined autistic group and the SNPs in each group were compared to SNPs in 2438 nonautistic controls. These analyses produced 5 sets of SNPs, i.e., QTLs, that were associated with specific subtypes of ASD (Tables 2-6). Finally, significant SNPs with Bonferroni-adjusted p-values ≦0.05 from each of the 5 separate subtype-dependent association analyses with QTL were combined into a single set containing 18 unique SNPs, and the association analysis was repeated using combined and subtyped ASD cases and 2438 controls.

Partial Replication of SNPs Between Subtypes of ASD

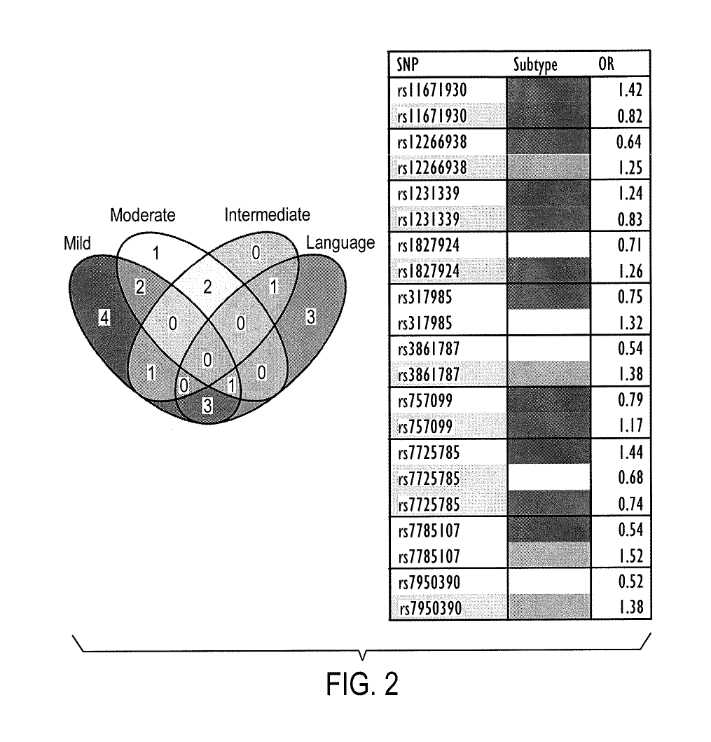

Table 7 shows the SNPs associated with each subtype of ASD that resulted from the final association analysis using the combined QTL and subgroups of ASD cases. Eighteen of the SNPs have p-values <0.05 even after using the stringent Bonferroni correction for multiple comparisons. Note that 10 of the SNPs, including rs317985, rs7785107, rs11671930, rs7950390, rs12266938, rs3861787, rs7725785, rs1827924, rs1231339, and rs757099, are associated with more than one subtype. Two of the replicated SNPs (rs317985 and rs7785107) are significant in two subtypes after Bonferroni adjustment (p<0.05), while the remaining 8 (shaded in Table 7) exhibit lower levels of significance (nominal p-values from 0.0037-0.051 or FDR_BH derived p-values of 0.0087-0.088) in the second (or third) subtype. Association of these QTL with more than one subtype of ASD serves as a replication for these 10 SNPs. Furthermore, the subtype-dependent differences in minor allele frequency and odds ratios associated with the shared SNPs demonstrate the ability of the subtyping method used in this study to separate ASD phenotypes that are genetically heterogeneous. FIG. 2 summarizes the extent of SNP overlap among the 4 subtypes and clearly demonstrates that the odds ratios are different for different subtypes that share the same SNP. All of the QTL associated with specific genes are present in noncoding (promoter or intronic) regions, or in intergenic regions. Interestingly, all but one of the SNPs residing within intergenic regions can be associated by band position to rare copy number variants (CNV) that have been recently identified for ASD (15). These are noted in Table 7.

Effect of ASD Subtyping on Association of Previously Identified SNPs within Chr5p14.1

Because none of the SNPs in Table 7 overlapped with those of the previously published genome-wide association study (9), we examined the association of the 6 SNPs that were reported in the published study with our ASD subphenotypes. Table 8 shows that only the “Moderate” ASD subtype (363 cases) is associated with two of the SNPs, with Bonferroni-adjusted p-values of 0.035 and 0.053. Interestingly, these 2 SNPs have the lowest combined p-value in the published study. The remaining 4 SNPs were suggestively significant with FDR_BH-adjusted p-values of 0.074 in this subtype. The combined cases (1867 in all) as well as the other 3 ASD subtypes show no association with any of the 6 SNPs even though there are more cases in each of these groups than in the Moderate group. This finding further illustrates the value of analyzing subphenotypes of ASD in genome-wide association analyses.

Pathway Analyses of SNP-Containing Genes

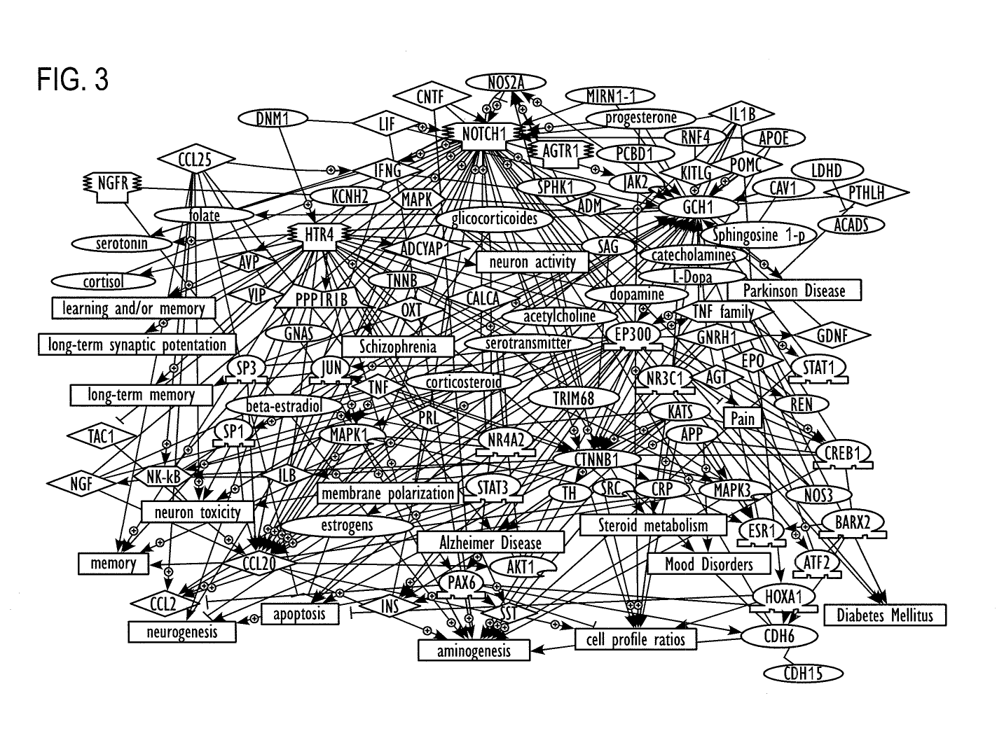

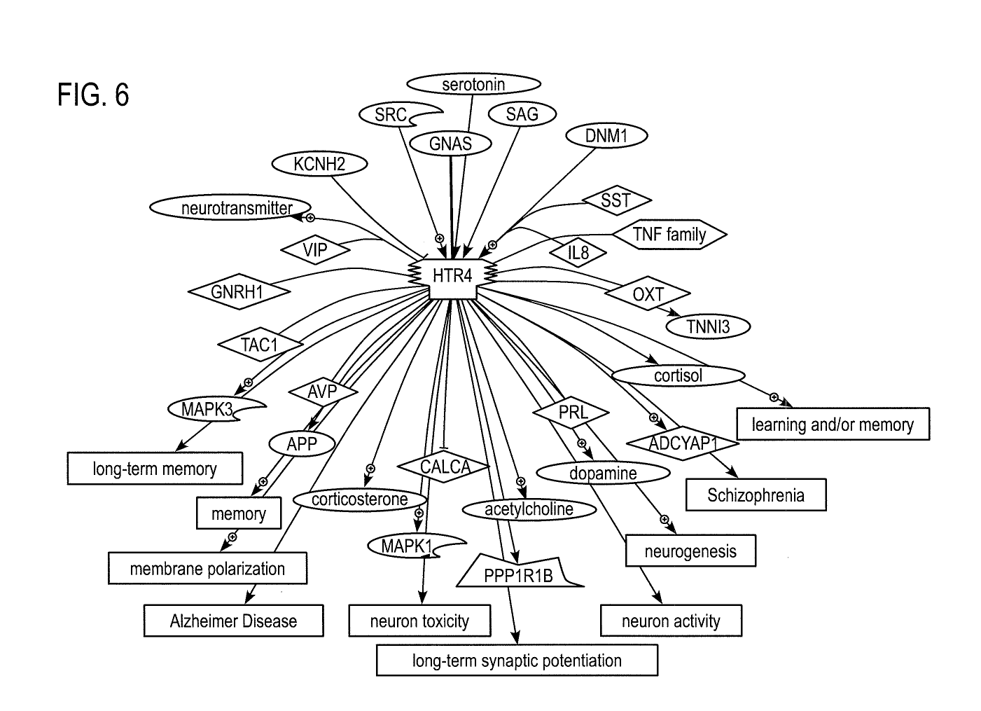

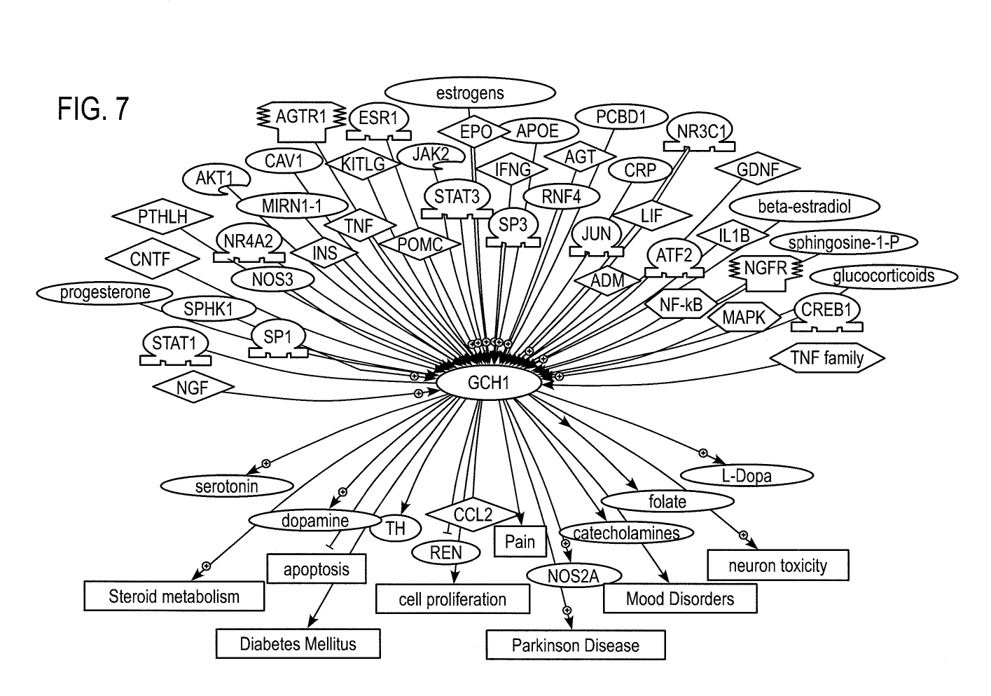

To obtain a better understanding of how the novel SNPs identified in this study potentially relate to the biology of autism, pathway analysis was conducted to develop a better sense of the relationships among the SNP-associated genes and their impact on higher level functions and diseases. FIG. 3 shows a gene network constructed using Pathway Studio 7 which includes seven of the 9 genes associated with SNPs found within gene promoters or introns. Of the 7 genes, HTR4 and GCH1 show the highest “connectivity” with other components within the network. The relationships between these two genes and other network components are illustrated in FIGS. 6 and 7. It is noted that many of the cellular and higher level processes in this network, such as neurogenesis, axonogenesis, steroid metabolism, cell proliferation, long-term synaptic potentiation, learning and memory are relevant to identified deficits in ASD.

Discussion

Previously, the inventor has shown that the autistic population can be divided into subgroups according to symptomatic profile through cluster analyses of severity scores from the ADI-R assessment for each individual with ASD (13). Three of the 4 resulting subgroups were shown to exhibit distinct, though partially overlapping, differential gene expression profiles, each relative to a group of nonautistic controls, implying that both unique and shared genes are associated with the respective phenotypes (14). Herein, the inventor applied the rationale and methods in subtyping individuals with ASD for this analysis of previously published genome-wide association data (9). The inventor employed quantitative trait association analyses to the >500,000 SNPs tested in order to prioritize SNPs that might correlate with a behavioral or symptomatic “trait” relevant to ASD. These quantitative traits for each individual with ASD were derived from the sums of severity scores from the ADI-R items that described severity of language impairment, deficits in nonverbal communication, impaired play skills, insistence on sameness/rituals, and delayed social development. The specific items used to establish each quantitative trait (listed in Table 9) were shown by flu and Steinberg to exhibit differential severity among the several subtypes of ASD (13). In the first (discovery) stage of the experiment, quantitative trait association analyses across the 5 selected traits produced a filtered set of 167 unique SNPs from the original 513,312 SNPs (Table 1). Subsequent association analyses of these QTL with both combined and subtyped individuals with ASD revealed 18 novel SNPs that were found to be highly significant (Bonferroni-adjusted p-value <0.05) in at least one subtype of autism (Table 7). Interestingly, many of the language QTL from Table 1 are strongly associated with the severely language-impaired ASD subtype. Of the 18 novel SNPs, 10 SNPs were replicated in at least one of the other subtypes. Two are replicated with Bonferroni-significant p-values while the other 8 are significant at a lower p-value (FDR-BH adjusted p-value <0.09). More significantly, different minor allele frequencies and odds ratios are associated with the SNPs that are associated with more than one subtype (see Table 7 and FIG. 2), thus reflecting the genetic heterogeneity between the ASD phenotypes, which is teased apart by the subtyping procedures employed here. It is noteworthy that no significant SNPs were identified when all 1867 individuals with ASD were analyzed against 2438 nonautistic controls, thus underscoring the importance of phenotypic subtyping to unearthing SNP associations with ASD.

By comparison, the original genome-wide study which was based on the combined analysis of 2503 cases and more than 7000 controls across 2 independent datasets in the “discovery” phase, identified 1 SNP that reached genome-wide significance and 5 additional SNPs of nominal significance in one intergenic region on chr5p14.1 that was located between 2 cadherin genes (9). In this study, the association of 2 of the previously identified SNPs was detected only in the “Moderate” subtype of ASD. Neither the other 3 subtypes nor the combined case group showed any association with these previously identified 6 SNPs. None of the 6 SNPs identified in the published study correlated with expression level of either cadherin (9 or 10) in the cortical brain of 93 genotyped human subjects. However, 2 of the SNPs found in the current study, are associated with the genes HTR4 and CCL25, which were found to be differentially expressed (FDR<5%) in lymphoblastoid cell lines from individuals with ASD in a previous study (14). This overlap of differentially expressed genes with those associated with at least some of the novel SNPs lends support to the functional relevance of these SNPs. Aside from the SNPs located within the promoter or intron regions of genes, the majority of the other significant SNPs (Table 7) are located in intergenic regions that are linked by band position to rare copy number variants (CNVs) that have been recently associated with autism (15). The presence of the identified QTL in ASD-related CNVs provides additional support for the relevance of these novel SNPs to ASD. The current study thus illustrates the advantage of utilizing both quantitative traits and defined ASD phenotypes in analyzing genome-wide genetic data from individuals with this complex disorder.

Biological Relevance of SNP-Containing Genes

To examine the biological processes and pathways that might be impacted by the SNP-associated genes, we performed pathway analyses on the genes using Pathway Studio 7 software. FIG. 3 shows that 7 of the 9 SNP-containing genes could be included in a gene network in which HTR4 and GCH1 are “hubs” connecting with many other genes, cellular processes and disorders (see FIGS. 6 and 7 for specific connections). As illustrated in FIG. 6, HTR4 [5-hydroxytryptamine (serotonin) receptor 4] regulates neurogenesis, long-term synaptic potentiation and, in turn, learning and memory, as well as the release of neurotransmitters (dopamine, acetylcholine), peptide hormones (AVP, OXT, PRL, VIP) and steroid compounds (cortisol, corticosterone). Thus, any alteration in the expression or function of this gene can be expected to have wide-ranging consequences on processes known to be affected by ASD. It is notable that one of the SNPs associated with HTR4, rs7725785, is associated with three ASD subtypes. However, the odds ratio is 1.44 for the severe language-impaired subtype while it is 0.68 and 0.74 for the moderate and mild subtypes, respectively. Interestingly, a reduction in HTR4 expression was observed only in the language-impaired subtype of ASD (14). Genetic variants in HTR4 have also been associated with schizophrenia (23), bipolar disorder (24) and attention deficit/hyperactivity disorder (25). More recently, a de novo translocation on chromosome 5 close to HTR4 has been identified in an autistic boy (26). The other hub gene, GCH1 [GTP cyclohydrolase I], is the rate-limiting enzyme in the de novo biosynthesis of tetrahydrobiopterin which is in turn required for the biosynthesis of folate, serotonin, dopamine, and catecholamines (FIG. 7). It is interesting to note that elevated expression of GCH1 has been implicated in mood disorders (27), while genetic polymorphisms or mutations in GCH1 have been associated with pain sensitivity (28-30), and dystonia (31), which are often associated with ASD. Although these genes are not likely to be causal for ASD, genetic polymorphisms in them may be associated with some of the comorbid symptoms or pathobiology of ASD.

Together, these analyses provide support for the biological relevance of these QTL to ASD and identify additional candidate genes for functional testing. Importantly, this study also reveals genetic biomarkers which not only may be used for diagnostic screening of ASD, but also offer the additional advantage of being associated with ASD subtypes that may be linked to specific and targeted therapies through pharmacogenomics studies. Finally, the association of different SNPs with the 4 subtypes of autism reinforces the idea that there are multiple genetic etiologies giving rise to the autistic spectrum, while the shared SNPs between different subtypes may reveal common genetic mechanisms responsible for core symptoms.

Summary

This study is the first to demonstrate the value of using a combination of quantitative trait analysis and subphenotyping of individuals with ASD to identify genetic variants (SNPs) that associate with specific behavioral phenotypes of ASD. It is noted that no Bonferroni-significant SNPs are detected when all 1867 autistic cases are combined into a single group and compared against 2438 non-autistic controls. Thus, even though the number of cases is lower in each of the subgroups, there is more power to detect statistically significant SNPs associated with the more homogeneous subgroups of ASD individuals than with the combined ASD population. Subtyping also creates separate case cohorts which are shown to replicate 10 of the novel SNPs identified in this study, thus providing a form of internal validation. Differences in minor allele frequencies of these 10 SNPs in the different cohorts further demonstrate the genetic heterogeneity among the subtypes. Together, these findings not only reveal novel subtype-dependent candidate genes for ASD, but also identify genetic markers for diagnostic screening and assessment of an individual's propensity or increased risk of having or developing an ASD.

TABLE 1
Quantitative trait loci for 5 ASD-associated traits: language impairment, deficits in nonverbal communication,
impaired play skills, insistence on sameness and rituals, and deficits in social development

| | | | | | | AA | | |
|---|---|---|---|---|---|---|---|---|
| | | | | | | change | | |
| SNP | SNP position | Band | Location | Gene | Alleles | (position) | UNADJ P | Trait |
| rs12407665 | chr1: 14070302 | 1p36.21 | CNV* | | C/G/T | | 4.16E−07 | Language |
| | | | | | | | | impairment |
| rs17828521 | chr6: 54328202 | 6p12.1 | Intron | TINAG | C/T | | 7.46E−07 | Language |
| | | | | | | | | impairment |
| rs9474831 | chr6: 54361040 | 6p12.1 | Intron | TINAG | C/T | | 1.09E−06 | Language |
| | | | | | | | | impairment |
| rs6454792 | chr6: 90743794 | 6q15 | Intron | BACH2 | A/G | | 1.41E−06 | Language |
| | | | | | | | | impairment |
| rs10183984 | chr2: 167181162 | 2q24.3 | CNV | | A/G | | 2.82E−06 | Language |
| | | | | | | | | impairment |
| rs11969265 | chr6: 90739765 | 6q15 | Intron | BACH2 | C/T | | 3.03E−06 | Language |
| | | | | | | | | impairment |
| rs1231339 | chr9: 25730863 | 9p21.2 | CNV | | A/C | | 3.20E−06 | Language |
| | | | | | | | | impairment |
| rs10806416 | chr6: 90755541 | 6q15 | Intron | BACH2 | C/T | | 3.38E−06 | Language |
| | | | | | | | | impairment |
| rs7785107 | chr7: 10247968 | 7p21.3 | CNV | | G/T | | 4.58E−06 | Language |
| | | | | | | | | impairment |
| rs2277049 | chr5: 147883281 | 5q33.1 | Intron | HTR4 | A/C | | 5.05E−06 | Language |
| | | | | | | | | impairment |
| rs757099 | chr9: 25727567 | 9p21.2 | CNV | | C/T | | 6.20E−06 | Language |
| | | | | | | | | impairment |
| rs7725785 | chr5: 147882896 | 5q33.1 | Intron | HTR4 | A/C | | 6.38E−06 | Language |
| | | | (boundary) | | | | | impairment |
| rs758158 | chr12: 1895465 | 12p13.33 | Intron | CACNA2D4 | A/G | | 6.69E−06 | Language |
| | | | | | | | | impairment |
| rs2287581 | chr5: 31341022 | 5p13.3 | Intron | CDH6 | A/G | | 6.76E−06 | Language |
| | | | (boundary) | | | | | impairment |
| rs17830215 | chr8: 1902159 | 8p23.3 | Promoter | KBTBD11 | C/T | | 7.65E−06 | Language |
| | | | | | | | | impairment |
| rs2180055 | chr22: 47722427 | 22q13.32 | CNV | | C/T | | 8.07E−06 | Language |
| | | | | | | | | impairment |
| rs12893752 | chr14: 25220350 | 14q12 | CNV | | C/T | | 8.74E−06 | Language |
| | | | | | | | | impairment |
| rs9941626 | chr2: 212059912 | 2q34 | Intron | ERBB4 | G/T | | 4.88E−07 | Nonverbal |
| | | | | | | | | deficits |
| rs13205238 | chr6: 413597 | 6p25.3 | CNV | | A/G | | 9.43E−07 | Nonverbal |
| | | | | | | | | deficits |
| rs11671930 | chr19: 8023308 | 19p13.2 | Promoter | CCL25 | C/T | | 1.25E−06 | Nonverbal |
| | | | | | | | | deficits |
| rs11229410 | chr11: 57926867 | 11q12.1 | Coding exon | OR5B3 | C/T | I/V (198) | 1.32E−06 | Nonverbal |
| | | | | | | | | deficits |
| rs11229413 | chr11: 57927314 | 11q12.1 | Coding exon | OR5B3 | A/G | W/R (49) | 2.07E−06 | Nonverbal |
| | | | | | | | | deficits |
| rs11229411 | chr11: 57926918 | 11q12.1 | Coding exon | OR5B3 | C/T | A/T (181) | 2.35E−06 | Nonverbal |
| | | | | | | | | deficits |
| rs11721070 | chr3: 72004997 | 3p13 | CNV | | C/T | | 2.55E−06 | Nonverbal |
| | | | | | | | | deficits |
| rs12466917 | chr2: 207427129 | 2q33.3 | — | | A/G | | 2.78E−06 | Nonverbal |
| | | | | | | | | deficits |
| rs13076171 | chr3: 71988841 | 3p13 | CNV | | A/G | | 3.12E−06 | Nonverbal |
| | | | | | | | | deficits |
| rs7930778 | chr11: 57792932 | 11q12.1 | Promoter | OR10W1 | C/T | | 4.09E−06 | Nonverbal |
| | | | | | | | | deficits |
| rs12962411 | chr18: 27645521 | 18q12.1 | CNV | | A/G | | 4.11E−06 | Nonverbal |
| | | | | | | | | deficits |
| rs12279895 | chr11: 57926572 | 11q12.1 | Coding exon | OR5B3 | C/T | K/R (296) | 4.53E−06 | Nonverbal |
| | | | | | | | | deficits |
| rs730168 | chr16: 73707776 | 16q23.1 | Intron | LDHD | A/G | | 4.63E−06 | Nonverbal |
| | | | | | | | | deficits |
| rs13021324 | chr2: 212058490 | 2q34 | Intron | ERBB4 | C/T | | 4.76E−06 | Nonverbal |
| | | | | | | | | deficits |
| rs564127 | chr7: 79734155 | 7q21.11 | CNV | | C/T | | 5.14E−06 | Nonverbal |
| | | | | | | | | deficits |
| rs1231339 | chr9: 25730863 | 9p21.2 | CNV | | A/C | | 5.78E−06 | Nonverbal |
| | | | | | | | | deficits |
| rs393076 | chr3: 162584791 | 3q26.1 | CNV | | A/G | | 5.99E−06 | Nonverbal |
| | | | | | | | | deficits |
| rs1938651 | chr11: 57869527 | 11q12.1 | CNV | | C/T | | 6.00E−06 | Nonverbal |
| | | | | | | | | deficits |
| rs11138895 | chr9: 82693023 | 9q21.31 | — | | A/C | | 6.21E−06 | Nonverbal |
| | | | | | | | | deficits |
| rs1938672 | chr11: 57885629 | 11q12.1 | Promoter | OR5B17 | C/T | | 6.29E−06 | Nonverbal |
| | | | | | | | | deficits |
| rs4804202 | chr19: 12579619 | 19p13.2 | Promoter | FU90396 | C/T | | 6.96E−06 | Nonverbal |
| | | | | | | | | deficits |
| rs665036 | chr1: 61053139 | 1p31.3 | CNV | | C/T | | 8.07E−06 | Nonverbal |
| | | | | | | | | deficits |
| rs4527692 | chr6: 21268426 | 6p22.3 | Intron | CDKAL1 | C/T | | 8.08E−06 | Nonverbal |
| | | | | | | | | deficits |
| rs519514 | chr7: 78294084 | 7q21.11 | Intron | MAGI2 | C/T | | 8.45E−06 | Nonverbal |
| | | | | | | | | deficits |
| rs3133855 | chr11: 120090061 | 11q23.3 | Intron | GRIK4 | A/G | | 9.91E−06 | Nonverbal |
| | | | | | | | | deficits |
| rs1938670 | chr11: 57884219 | 11q12.1 | Promoter | OR5B17 | A/G | | 9.95E−06 | Nonverbal |
| | | | | | | | | deficits |
| rs13205238 | chr6: 413597 | 6p25.3 | CNV | | A/G | | 5.46E−08 | Play skills |
| rs1996893 | chr9: 14880268 | 9p22.3 | Intron | FREM1 | C/T | | 1.44E−07 | Play skills |
| rs12606567 | chr18: 48408860 | 18q21.2 | Intron | DCC | C/T | | 5.59E−07 | Play skills |
| rs3769845 | chr2: 230863948 | 2q37.1 | Intron | SP140 | C/T | | 6.19E−07 | Play skills |
| rs2422675 | chr20: 1940396 | 20p13 | CNV | | A/G | | 9.81E−07 | Play skills |
| rs4798405 | chr18: 5732206 | 18p11.31 | CNV | | A/G | | 1.02E−06 | Play skills |
| rs10040891 | chr5: 2166288 | 5p15.33 | CNV | | C/T | | 1.16E−06 | Play skills |
| rs8181738 | chr12: 23095146 | 12p12.1 | — | | G/T | | 1.21E−06 | Play skills |
| rs11950809 | chr5: 2166672 | 5p15.33 | CNV | | A/C | | 1.48E−06 | Play skills |
| rs11627027 | chr14: 93440170 | 14q32.13 | — | | C/T | | 1.52E−06 | Play skills |
| rs1930 | chr2: 80368183 | 2p12 | Intron | CTNNA2 | C/T | | 1.56E−06 | Play skills |
| rs4894734 | chr3: 177013204 | 3q26.31 | Downstream | NAALADL2 | A/C | | 1.60E−06 | Play skills |
| rs1482930 | chr15: 79651018 | 15q25.2 | CNV | | A/G | | 2.21E−06 | Play skills |
| rs11671930 | chr19: 8023308 | 19p13.2 | Promoter | CCL25 | C/T | | 2.32E−06 | Play skills |
| rs4980777 | chr11: 68927459 | 11q13.2 | CNV | | A/G | | 2.35E−06 | Play skills |
| rs1481513 | chr8: 79171163 | 8q21.12 | CNV | | C/T | | 2.45E−06 | Play skills |
| rs10987251 | chr9: 128142815 | 9q33.3 | Intron | C9orf28 | A/G | | 2.54E−06 | Play skills |
| | | | (boundary) | | | | | |
| rs2151206 | chr9: 14844072 | 9p22.3 | Intron | FREM1 | A/G | | 2.55E−06 | Play skills |
| rs2044747 | chr14: 42393956 | 14q21.2 | CNV | | G/T | | 2.58E−06 | Play skills |
| rs1440423 | chr5: 34425616 | 5p13.2 | CNV | | C/T | | 2.98E−06 | Play skills |
| rs4745257 | chr9: 75582705 | 9q21.13 | CNV | | A/C | | 3.06E−06 | Play skills |
| rs2779499 | chr9: 14840816 | 9p22.3 | Intron | FREM1 | C/T | | 3.25E−06 | Play skills |
| rs1796028 | chr2: 96982680 | 2q11.2 | CNV | | A/G | | 3.47E−06 | Play skills |
| rs1888156 | chr9: 128142661 | 9q33.3 | Coding exon | C9orf28 | A/G | T/T (45) | 3.52E−06 | Play skills |
| rs6734788 | chr2: 208801314 | 2q33.3 | Downstream | IDH1 | C/T | | 3.61E−06 | Play skills |
| rs7605424 | chr2: 80155080 | 2p12 | Intron | CTNNA2 | A/G | | 3.71E−06 | Play skills |
| rs4627775 | chr3: 106308609 | 3q13.11 | — | | G/T | | 3.75E−06 | Play skills |
| rs5009527 | chr4: 184601958 | 4q35.1 | Promoter | CARF | C/T | | 3.88E−06 | Play skills |
| rs1796045 | chr2: 96986737 | 2q11.2 | CNV | | C/T | | 4.05E−06 | Play skills |
| rs1863080 | chr2: 36379940 | 2p22.3 | CNV | | A/C | | 4.26E−06 | Play skills |
| rs7337921 | chr13: 23381053 | 13q12.12 | Intron | FLJ46358 | A/G | | 4.27E−06 | Play skills |
| rs6452136 | chr5: 23722770 | 5p14.2 | — | | C/T | | 4.42E−06 | Play skills |
| rs2168709 | chr18: 56096085 | 18q21.32 | CNV | | C/T | | 4.42E−06 | Play skills |
| rs4386512 | chr3: 177012791 | 3q26.31 | Downstream | NAALADL2 | C/T | | 4.53E−06 | Play skills |
| rs12614870 | chr2: 126505161 | 2q14.3 | CNV | | A/G | | 4.54E−06 | Play skills |
| rs10491885 | chr9: 28507137 | 9p21.1 | Intron | LRRN6C | C/T | | 4.58E−06 | Play skills |
| rs4646421 | chr15: 72803245 | 15q24.1 | Intron | CYP1A1 | C/T | | 4.99E−06 | Play skills |
| rs4894733 | chr3: 177013074 | 3q26.31 | Downstream | NAALADL2 | G/T | | 5.10E−06 | Play skills |
| rs7944323 | chr11: 78903676 | 11q14.1 | CNV | | G/T | | 5.26E−06 | Play skills |
| rs6791089 | chr3: 176998117 | 3q26.31 | Intron | NAALADL2 | A/C | | 5.45E−06 | Play skills |
| rs11229410 | chr11: 57926867 | 11q12.1 | Coding exon | OR5B3 | C/T | I/V (198) | 5.51E−06 | Play skills |
| rs17770167 | chr5: 129679109 | 5q23.3 | CNV | | A/G | | 5.59E−06 | Play skills |
| rs6698676 | chr1: 84528583 | 1p31.1 | Promoter | SAMD13 | G/T | | 5.76E−06 | Play skills |
| rs11664663 | chr18: 44397104 | 18q21.1 | Intron | KIAA0427 | C/T | | 5.79E−06 | Play skills |
| rs6482516 | chr10: 18879356 | 10p12.33 | Intron | NSUN6 | A/G | | 5.79E−06 | Play skills |
| rs11082277 | chr18: 38304361 | 18q12.3 | CNV | | A/G | | 6.00E−06 | Play skills |
| rs6988293 | chr8: 121303479 | 8q24.12 | Intron | COL14A1 | A/G | | 6.00E−06 | Play skills |
| rs6974649 | chr7: 130463637 | 7q32.3 | CNV | | A/C | | 6.11E−06 | Play skills |
| rs730168 | chr16: 73707776 | 16q23.1 | Intron | LDHD | A/G | | 6.51E−06 | Play skills |
| rs1461710 | chr18: 38197191 | 18q12.3 | CNV | | A/G | | 6.67E−06 | Play skills |
| rs9941626 | chr2: 212059912 | 2q34 | Intron | ERBB4 | G/T | | 7.03E−06 | Play skills |
| rs3745651 | chr19: 12553001 | 19p13.2 | Coding exon | ZNF490 | C/T | H/H (296) | 7.49E−06 | Play skills |
| rs9536962 | chr13: 54576937 | 13q21.1 | CNV | | A/G | | 7.66E−06 | Play skills |
| rs7529505 | chr1: 99135144 | 1p21.3 | Intron | PAP2D | C/T | | 7.74E−06 | Play skills |
| rs9342127 | chr6: 88633834 | 6q15 | — | | A/G | | 8.03E−06 | Play skills |
| rs1554547 | chr12: 97892279 | 12q23.1 | Intron | ANKS1B | A/G | | 8.24E−06 | Play skills |
| rs9508456 | chr13: 28914362 | 13q12.3 | Intron | KIAA0774 | C/T | | 8.40E−06 | Play skills |
| rs2078520 | chr2: 80246156 | 2p12 | Intron | CTNNA2 | A/G | | 8.47E−06 | Play skills |
| rs9569991 | chr13: 33121745 | 13ql3.2 | — | | A/G | | 8.70E−06 | Play skills |
| rs3825597 | chr14: 51488595 | 14q22.1 | Intron | GNG2 | A/G | | 8.80E−06 | Play skills |
| rs3754741 | chr2: 173761465 | 2q31.1 | Intron | ZAK | C/T | | 9.36E−06 | Play skills |
| rs2250595 | chr12: 45642394 | 12q13.11 | — | | A/G | | 9.64E−06 | Play skills |
| rs1055518 | chr10: 73177651 | 10q22.1 | 3′ UTR | C10orf54 | A/G | | 9.72E−06 | Play skills |
| rs2600685 | chr2: 175335294 | 2q31.1 | Intron | CHRNA1 | A/G | | 9.99E−06 | Play skills |
| rs164187 | chr1: 160615261 | 1q23.3 | Promoter | C1orf111 | C/T | | 1.61E−07 | Sameness- |
| | | | | | | | | rituals |
| rs3809854 | chr17: 42384293 | 17q21.32 | CNV | | C/T | | 6.28E−07 | Sameness- |
| | | | | | | | | rituals |
| rs3804967 | chr3: 7479914 | 3p26.1 | Intron | GRM7 | A/G | | 1.11E−06 | Sameness- |
| | | | | | | | | rituals |
| rs3804968 | chr3: 7477700 | 3p26.1 | Intron | GRM7 | A/G | | 1.90E−06 | Sameness- |
| | | | | | | | | rituals |
| rs317985 | chr5: 66773558 | 5q13.1 | CNV | | A/G | | 2.24E−06 | Sameness- |
| | | | | | | | | rituals |
| rs9634811 | chr13: 47362176 | 13q14.2 | — | | A/G | | 2.39E−06 | Sameness- |
| | | | | | | | | rituals |
| rs7819605 | chr8: 67423026 | 8q13.1 | CNV | | A/G | | 2.68E−06 | Sameness- |
| | | | | | | | | rituals |
| rs7950390 | chr11: 4587928 | 11p15.4 | Promoter | TRIM68 | G/T | | 2.87E−06 | Sameness- |
| | | | | | | | | rituals |
| rs4436186 | chr9: 72323371 | 9q21.11 | CNV | | A/G | | 3.00E−06 | Sameness- |
| | | | | | | | | rituals |
| rs4838964 | chr1: 113094181 | 1p13.2 | CNV | | A/G | | 3.27E−06 | Sameness- |
| | | | | | | | | rituals |
| rs1827924 | chr2: 228377979 | 2q36.3 | Promoter | CCL20 | A/G | | 3.32E−06 | Sameness- |
| | | | | | | | | rituals |
| rs7699496 | chr4: 43931165 | 4p13 | Intron | KCTD8 | A/G | | 3.59E−06 | Sameness- |
| | | | | | | | | rituals |
| rs3861787 | chr11: 4604346 | 11p15.4 | CNV | | G/T | | 4.08E−06 | Sameness- |
| | | | | | | | | rituals |
| rs6782718 | chr3: 7462776 | 3p26.1 | Intron | GRM7 | A/G | | 4.64E−06 | Sameness- |
| | | | | | | | | rituals |
| rs11038286 | chr11: 45032402 | 11p11.2 | CNV | | A/G | | 4.71E−06 | Sameness- |
| | | | | | | | | rituals |
| rs693442 | chr13: 100725498 | 13q33.1 | Intron | VGCNL1 | A/G | | 4.74E−06 | Sameness- |
| | | | | | | | | rituals |
| rs1452885 | chr12: 43231117 | 12q12 | Intron | NELL2 | A/G | | 5.21E−06 | Sameness- |
| | | | | | | | | rituals |
| rs17599556 | chr4: 44013295 | 4p13 | Intron | KCTD8 | A/C | | 5.28E−06 | Sameness- |
| | | | | | | | | rituals |
| rs185425 | chr5: 57665978 | 5q11.2 | CNV | | C/T | | 5.40E−06 | Sameness- |
| | | | | | | | | rituals |
| rs11035240 | chr11: 39332276 | 11p12 | CNV | | C/T | | 5.56E−06 | Sameness- |
| | | | | | | | | rituals |
| rs9693369 | chr8: 138599449 | 8q24.23 | — | | C/T | | 6.25E−06 | Sameness- |
| | | | | | | | | rituals |
| rs10781238 | chr9: 76384432 | 9q21.13 | Intron | RORB | C/T | | 6.33E−06 | Sameness- |
| | | | | | | | | rituals |
| rs9568011 | chr13: 47606812 | 13q14.2 | — | | C/T | | 7.21E−06 | Sameness- |
| | | | | | | | | rituals |
| rs11682846 | chr2: 156716949 | 2q24.1 | CNV | | C/T | | 7.53E−06 | Sameness- |
| | | | | | | | | rituals |
| rs7650071 | chr3: 137459995 | 3q22.3 | Intron | PCCB | A/G | | 7.79E−06 | Sameness- |
| | | | | | | | | rituals |
| rs2574852 | chr17: 73003467 | 17q25.3 | Intron | SEPT9 | A/G | | 8.14E−06 | Sameness- |
| | | | | | | | | rituals |
| rs11914753 | chr3: 173791734 | 3q26.31 | CNV | | C/T | | 8.16E−06 | Sameness- |
| | | | | | | | | rituals |
| rs2469183 | chr15: 84992924 | 15q25.3 | CNV | | G/T | | 8.21E−06 | Sameness- |
| | | | | | | | | rituals |
| rs274646 | chr5: 6841282 | 5p15.31 | CNV | | C/T | | 8.30E−06 | Sameness- |
| | | | | | | | | rituals |
| rs13096022 | chr3: 7465129 | 3p26.1 | Intron | GRM7 | A/G | | 8.76E−06 | Sameness- |
| | | | | | | | | rituals |
| rs17738966 | chr14: 54371969 | 14q22.2 | Downstream | GCH1 | A/G | | 9.01E−06 | Sameness- |
| | | | | | | | | rituals |
| rs6461176 | chr7: 15453420 | 7p21.1 | Intron | FLJ16237 | A/C | | 9.06E−06 | Sameness- |
| | | | | | | | | rituals |
| rs13205238 | chr6: 413597 | 6p25.3 | CNV | | A/G | | 1.19E−10 | Social |
| | | | | | | | | development |
| rs11138895 | chr9: 82693023 | 9q21.31 | CNV | | A/C | | 3.92E−07 | Social |
| | | | | | | | | development |
| rs4809918 | chr20: 50734607 | 20q13.2 | — | | A/G | | 4.88E−07 | Social |
| | | | | | | | | development |
| rs9479482 | chr6: 150399705 | 6q25.1 | — | | C/T | | 6.68E−07 | Social |
| | | | | | | | | development |
| rs1294264 | chr1: 231548273 | 1q42.2 | Intron | KIAA1804 | C/T | | 9.59E−07 | Social |
| | | | | | | | | development |
| rs10788819 | chr1: 150161717 | 1q21.3 | — | | C/T | | 1.07E−06 | Social |
| | | | | | | | | development |
| rs4959923 | chr6: 412773 | 6p25.3 | CNV | | A/G | | 1.21E−06 | Social |
| | | | | | | | | development |
| rs4905110 | chr14: 93432083 | 14q32.13 | — | | A/G | | 1.21E−06 | Social |
| | | | | | | | | development |
| rs721087 | chr2: 4942340 | 2p25.2 | CNV | | C/T | | 1.30E−06 | Social |
| | | | | | | | | development |
| rs12266938 | chr10: 3852940 | 10p15.1 | CNV | | C/T | | 1.50E−06 | Social |
| | | | | | | | | development |
| rs10874468 | chr2: 96959718 | 2q11.2 | CNV | | A/G | | 1.66E−06 | Social |
| | | | | | | | | development |
| rs13384439 | chr2: 187500106 | 2q32.1 | CNV | | A/G | | 2.11E−06 | Social |
| | | | | | | | | development |
| rs4416176 | chr2: 26536150 | 2p23.3 | Intron | OTOF | C/T | | 2.18E−06 | Social |
| | | | | | | | | development |
| rs10519124 | chr2: 67819501 | 2p14 | — | | A/G | | 2.22E−06 | Social |
| | | | | | | | | development |
| rs12962411 | chr18: 27645521 | 18q12.1 | CNV | | A/G | | 2.26E−06 | Social |
| | | | | | | | | development |
| rs6022029 | chr20: 50708572 | 20q13.2 | CNV | | A/G | | 2.32E−06 | Social |
| | | | | | | | | development |
| rs11627027 | chr14: 93440170 | 14q32.13 | — | | C/T | | 2.47E−06 | Social |
| | | | | | | | | development |
| rs6022039 | chr20: 50726331 | 20q13.2 | CNV | | C/T | | 2.98E−06 | Social |
| | | | | | | | | development |
| rs10886048 | chr10: 118928872 | 10q25.3 | CNV | | C/T | | 3.85E−06 | Social |
| | | | | | | | | development |
| rs4873815 | chr8: 144796206 | 8q24.3 | Promoter | ZNF623 | C/T | | 4.31E−06 | Social |
| | | | | | | | | development |
| rs4832481 | chr2: 16986218 | 2p24.3 | CNV | | A/C | | 4.48E−06 | Social |
| | | | | | | | | development |
| rs3809282 | chr12: 110192180 | 12q24.11 | Intron | CUTL2 | A/G | | 4.55E−06 | Social |
| | | | | | | | | development |
| rs1554547 | chr12: 97892279 | 12q23.1 | Intron | ANKS1B | A/G | | 4.56E−06 | Social |
| | | | | | | | | development |
| rs2297172 | chr9: 71563166 | 9q21.11 | Intron | PTAR1 | C/T | | 4.57E−06 | Social |
| | | | | | | | | development |
| rs2255313 | chr12: 102773924 | 12q23.3 | CNV | | C/T | | 4.60E−06 | Social |
| | | | | | | | | development |
| rs2627468 | chr8: 3812607 | 8p23.2 | Intron | CSMD1 | C/T | | 4.87E−06 | Social |
| | | | | | | | | development |
| rs12183587 | chr6: 150396301 | 6q25.1 | — | | G/T | | 5.77E−06 | Social |
| | | | | | | | | development |
| rs10305860 | chr4: 148625337 | 4q31.23 | Intron | EDNRA | A/G | | 5.91E−06 | Social |
| | | | | | | | | development |
| rs30746 | chr5: 135366157 | 5q31.1 | CNV | | A/G | | 6.10E−06 | Social |
| | | | | | | | | development |
| rs11138885 | chr9: 82678684 | 9q21.31 | — | | C/T | | 6.58E−06 | Social |
| | | | | | | | | development |
| rs1294293 | chr1: 231536935 | 1q42.2 | Intron | KIAA1804 | A/C | | 6.69E−06 | Social |
| | | | | | | | | development |
| rs12115722 | chr9: 133418328 | 9q34.13 | CNV | | G/T | | 6.94E−06 | Social |
| | | | | | | | | development |
| rs6698676 | chr1: 84528583 | 1p31.1 | Promoter | SAMD13 | G/T | | 7.87E−06 | Social |
| | | | | | | | | development |
| rs10997162 | chr10: 67906707 | 10q21.3 | Intron | CTNNA3 | G/T | | 7.93E−06 | Social |
| | | | | | | | | development |
| rs4646421 | chr15: 72803245 | 15q24.1 | Intron | CYP1A1 | C/T | | 8.12E−06 | Social |
| | | | | | | | | development |
| rs4778640 | chr15: 79391186 | 15q25.1 | Downstream | STARD5 | A/G | | 8.25E−06 | Social |
| | | | | | | | | development |
| rs10110252 | chr8: 17424455 | 8p22 | CNV | | C/T | | 8.56E−06 | Social |
| | | | | | | | | development |
| rs1996893 | chr9: 14880268 | 9p22.3 | Intron | FREM1 | C/T | | 8.59E−06 | Social |
| | | | | | | | | development |
| rs12811136 | chr12: 131603653 | 12q24.33 | CNV | | C/T | | 9.18E−06 | Social |
| | | | | | | | | development |
| rs17192980 | chr5: 7030768 | 5p15.31 | CNV | | C/T | | 9.29E−06 | Social |
| | | | | | | | | development |
| rs4811895 | chr20: 55639715 | 20q13.31 | — | | A/G | | 9.45E−06 | Social |
| | | | | | | | | development |
| rs2519866 | chr17: 27859883 | 17q11.2 | Intron | MYO1D | A/G | | 9.58E−06 | Social |
| | | | | | | | | development |
| rs2779499 | chr9: 14840816 | 9p22.3 | Intron | FREM1 | C/T | | 9.59E−06 | Social |
| | | | | | | | | development |
| rs2151206 | chr9: 14844072 | 9p22.3 | Intron | FREM1 | A/G | | 9.88E−06 | Social |
| | | | | | | | | development |

TABLE 2
Language QTL associated with ASD subtypes

| SNP | SNP position | Band | Location | Gene | UNADJ P | FDR_BH | BONF | Subtype |
|---|---|---|---|---|---|---|---|---|
| rs2277049 | chr5: 147883281 | 5q33.1 | Intron | HTR4 | 0.00021 | 0.00154 | 0.00362 | Language-impaired |
| rs757099 | chr9: 25727567 | 9p21.2 | CNV | | 0.00032 | 0.00154 | 0.00546 | ″ |
| rs7785107 | chr7: 10247968 | 7p21.3 | CNV | | 0.00035 | 0.00154 | 0.00594 | ″ |
| rs7725785 | chr5: 147882896 | 5q33.1 | Intron | HTR4 | 0.00036 | 0.00154 | 0.00618 | ″ |
| | | | (boundary) | | | | | |
| rs2287581 | chr5: 31341022 | 5p13.3 | Intron | CDH6 | 0.00065 | 0.00193 | 0.01111 | ″ |
| | | | (boundary) | | | | | |
| rs1231339 | chr9: 25730863 | 9p21.2 | CNV | | 0.00068 | 0.00193 | 0.01157 | ″ |
| rs2180055 | chr22: 47722427 | 22q13.32 | CNV | | 0.00148 | 0.00359 | 0.02515 | ″ |
| rs758158 | chr12: 1895465 | 12p13.33 | Intron | CACNA2D4 | 0.00316 | 0.00672 | 0.05375 | ″ |
| rs17830215 | chr8: 1902159 | 8p23.3 | Promoter | KBTBD11 | 0.00435 | 0.00821 | 0.07386 | ″ |
| rs10183984 | chr2: 167181162 | 2q24.3 | CNV | | 0.00596 | 0.01013 | 0.10130 | ″ |
| rs11969265 | chr6: 90739765 | 6q15 | Intron | BACH2 | 0.00800 | 0.01236 | 0.13590 | ″ |
| rs9474831 | chr6: 54361040 | 6p12.1 | Intron | TINAG | 0.00993 | 0.01406 | 0.16880 | ″ |
| rs17828521 | chr6: 54328202 | 6p12.1 | Intron | TINAG | 0.01163 | 0.01454 | 0.19770 | ″ |
| rs10806416 | chr6: 90755541 | 6q15 | Intron | BACH2 | 0.01198 | 0.01454 | 0.20360 | ″ |
| rs6454792 | chr6: 90743794 | 6q15 | Intron | BACH2 | 0.03486 | 0.03951 | 0.59270 | ″ |
| rs12893752 | chr14: 25220350 | 14q12 | CNV | | 0.09178 | 0.09751 | 1.00000 | ″ |
| rs7785107 | chr7: 10247968 | 7p21.3 | CNV | | 0.00156 | 0.02650 | 0.02650 | Intermediate |
| rs12407665 | chr1: 14070302 | 1p36.21 | CNV | | 0.00055 | 0.00934 | 0.00934 | Mild |
| rs12893752 | chr14: 25220350 | 14q12 | CNV | | 0.00677 | 0.05756 | 0.11510 | ″ |
| rs6454792 | chr6: 90743794 | 6q15 | Intron | BACH2 | 0.01371 | 0.07472 | 0.23300 | ″ |
| rs1231339 | chr9: 25730863 | 9p21.2 | CNV | | 0.02154 | 0.07472 | 0.36620 | ″ |
| rs9474831 | chr6: 54361040 | 6p12.1 | Intron | TINAG | 0.02198 | 0.07472 | 0.37360 | ″ |
| rs10183984 | chr2: 167181162 | 2q24.3 | CNV | | 0.03564 | 0.09278 | 0.60590 | ″ |
| rs17828521 | chr6: 54328202 | 6p12.1 | Intron | TINAG | 0.03821 | 0.09278 | 0.64950 | ″ |
| rs757099 | chr9: 25727567 | 9p21.2 | CNV | | 0.04414 | 0.09379 | 0.75030 | ″ |
| rs7725785 | chr5: 147882896 | 5q33.1 | Intron | HTR4 | 0.05082 | 0.09599 | 0.86390 | ″ |
| | | | (boundary) | | | | | |

TABLE 3
Nonverbal communication QTL associated with ASD subtypes

| SNP | SNP Position | Band | Location | Gene | UNADJ | FDR_BH | BONF | Subtype |
|---|---|---|---|---|---|---|---|---|
| rs1231339 | chr9: 25730863 | 9p21.2 | CNV | | 0.00068 | 0.01769 | 0.01769 | Language-impaired |
| rs519514 | chr7: 78294084 | 7q21.11 | Intron | MAGI2 | 0.00305 | 0.03965 | 0.07930 | ″ |
| rs11138895 | chr9: 82693023 | 9q21.31 | — | | 0.00463 | 0.04012 | 0.12030 | ″ |
| rs564127 | chr7: 79734155 | 7q21.11 | CNV | | 0.00650 | 0.04226 | 0.16900 | ″ |
| rs12466917 | chr2: 207427129 | 2q33.3 | — | | 0.01593 | 0.08282 | 0.41410 | ″ |
| rs11229411 | chr11: 57926918 | 11q12.1 | Coding exon | OR5B3 | 0.00957 | 0.08976 | 0.24880 | Intermediate |
| rs11229413 | chr11: 57927314 | 11q12.1 | Coding exon | OR5B3 | 0.01018 | 0.08976 | 0.26460 | ″ |
| rs12279895 | chr11: 57926572 | 11q12.1 | Coding exon | OR5B3 | 0.01079 | 0.08976 | 0.28060 | ″ |
| rs11229410 | chr11: 57926867 | 11q12.1 | Coding exon | OR5B3 | 0.01381 | 0.08976 | 0.35900 | ″ |
| rs1938670 | chr11: 57884219 | 11q12.1 | Promoter | OR5B17 | 0.01761 | 0.09159 | 0.45790 | ″ |
| rs1938651 | chr11: 57869527 | 11q12.1 | CNV | | 0.00458 | 0.05114 | 0.11900 | Moderate |
| rs1938672 | chr11: 57885629 | 11q12.1 | Promoter | OR5B17 | 0.00600 | 0.05114 | 0.15600 | ″ |
| rs11229410 | chr11: 57926867 | 11q12.1 | Coding exon | OR5B3 | 0.00841 | 0.05114 | 0.21860 | ″ |
| rs1938670 | chr11: 57884219 | 11q12.1 | Promoter | OR5B17 | 0.00939 | 0.05114 | 0.24410 | ″ |
| rs11229413 | chr11: 57927314 | 11q12.1 | Coding exon | OR5B3 | 0.01123 | 0.05114 | 0.29190 | ″ |
| rs11229411 | chr11: 57926918 | 11q12.1 | Coding exon | OR5B3 | 0.01227 | 0.05114 | 0.31900 | ″ |
| rs12279895 | chr11: 57926572 | 11q12.1 | Coding exon | OR5B3 | 0.01377 | 0.05114 | 0.35800 | ″ |
| rs9941626 | chr2: 212059912 | 2q34 | Intron | ERBB4 | 0.01704 | 0.05539 | 0.44310 | ″ |
| rs13021324 | chr2: 212058490 | 2q34 | Intron | ERBB4 | 0.02878 | 0.08314 | 0.74830 | ″ |
| rs7930778 | chr11: 57792932 | 11q12.1 | Promoter | OR10W1 | 0.03410 | 0.08866 | 0.88660 | ″ |
| rs730168 | chr16: 73707776 | 16q23.1 | Intron | LDHD | 0.00015 | 0.00363 | 0.00396 | Mild |
| rs11671930 | chr19: 8023308 | 19p13.2 | Promoter | CCL25 | 0.00028 | 0.00363 | 0.00726 | ″ |
| rs13205238 | chr6: 413597 | 6p25.3 | CNV | | 0.00331 | 0.02867 | 0.08601 | ″ |
| rs12962411 | chr18: 27645521 | 18q12.1 | CNV | | 0.01071 | 0.05215 | 0.27840 | ″ |
| rs393076 | chr3: 162584791 | 3q26.1 | CNV | | 0.01429 | 0.05215 | 0.37150 | ″ |
| rs11229411 | chr11: 57926918 | 11q12.1 | Coding exon | OR5B3 | 0.01459 | 0.05215 | 0.37940 | ″ |
| rs11229413 | chr11: 57927314 | 11q12.1 | Coding exon | OR5B3 | 0.01558 | 0.05215 | 0.40500 | ″ |
| rs11229410 | chr11: 57926867 | 11q12.1 | Coding exon | OR5B3 | 0.01908 | 0.05215 | 0.49610 | ″ |
| rs4804202 | chr19: 12579619 | 19p13.2 | Promoter | FLJ90396 | 0.02031 | 0.05215 | 0.52820 | ″ |
| rs1231339 | chr9: 25730863 | 9p21.2 | CNV | | 0.02154 | 0.05215 | 0.56000 | ″ |
| rs1938670 | chr11: 57884219 | 11q12.1 | Promoter | OR5B17 | 0.02206 | 0.05215 | 0.57370 | ″ |
| rs12279895 | chr11: 57926572 | 11q12.1 | Coding exon | OR5B3 | 0.02521 | 0.05298 | 0.65540 | ″ |
| rs11138895 | chr9: 82693023 | 9q21.31 | — | | 0.02649 | 0.05298 | 0.68870 | ″ |
| rs1938651 | chr11: 57869527 | 11q12.1 | CNV | | 0.02881 | 0.05350 | 0.74900 | ″ |
| rs9941626 | chr2: 212059912 | 2q34 | Intron | ERBB4 | 0.03374 | 0.05554 | 0.87720 | ″ |
| rs1938672 | chr11: 57885629 | 11q12.1 | Promoter | OR5B17 | 0.03418 | 0.05554 | 0.88860 | ″ |
| rs4527692 | chr6: 21268426 | 6p22.3 | Intron | CDKAL1 | 0.03755 | 0.05744 | 0.97640 | ″ |
| rs665036 | chr1: 61053139 | 1p31.3 | CNV | | 0.04121 | 0.05952 | 1.00000 | ″ |
| rs7930778 | chr11: 57792932 | 11q12.1 | Promoter | OR10W1 | 0.04635 | 0.06342 | 1.00000 | ″ |
| rs13021324 | chr2: 212058490 | 2q34 | Intron | ERBB4 | 0.05686 | 0.07059 | 1.00000 | ″ |
| rs3133855 | chr11: 120090061 | 11q23.3 | Intron | GRIK4 | 0.05701 | 0.07059 | 1.00000 | ″ |

TABLE 4
Play skills QTL associated with ASD subtypes

| SNP | SNP Position | Band | Location | Gene | UNADJ P | FDR_BH | BONF | Subtype |
|---|---|---|---|---|---|---|---|---|
| rs3754741 | chr2: 173761465 | 2q31.1 | Intron | ZAK | 0.00160 | 0.06409 | 0.10250 | Language-impaired |
| rs9569991 | chr13: 33121745 | 13q13.2 | — | | 0.00252 | 0.06409 | 0.16140 | ″ |
| rs4798405 | chr18: 5732206 | 18p11.31 | CNV | | 0.00300 | 0.06409 | 0.19230 | ″ |
| rs8181738 | chr12: 23095146 | 12p12.1 | — | | 0.00454 | 0.07264 | 0.29060 | ″ |
| rs1554547 | chr12: 97892279 | 12q23.1 | Intron | ANKS1B | 0.00676 | 0.08647 | 0.43240 | ″ |
| rs1481513 | chr8: 79171163 | 8q21.12 | CNV | | 0.00837 | 0.08927 | 0.53560 | ″ |
| rs730168 | chr16: 73707776 | 16q23.1 | Intron | LDHD | 0.00015 | 0.00595 | 0.00975 | Mild |
| rs6482516 | chr10: 18879356 | 10p12.33 | Intron | NSUN6 | 0.00019 | 0.00595 | 0.01230 | ″ |
| rs11671930 | chr19: 8023308 | 19p13.2 | Promoter | CCL25 | 0.00028 | 0.00595 | 0.01786 | ″ |
| rs11082277 | chr18: 38304361 | 18q12.3 | CNV | | 0.00165 | 0.02209 | 0.10530 | ″ |
| rs6698676 | chr1: 84528583 | 1p31.1 | Promoter | SAMD13 | 0.00173 | 0.02209 | 0.11050 | ″ |
| rs1461710 | chr18: 38197191 | 18q12.3 | CNV | | 0.00293 | 0.02646 | 0.18770 | ″ |
| rs3745651 | chr19: 12553001 | 19p13.2 | Coding exon | ZNF490 | 0.00300 | 0.02646 | 0.19200 | ″ |
| rs13205238 | chr6: 413597 | 6p25.3 | CNV | | 0.00331 | 0.02646 | 0.21170 | ″ |
| rs4386512 | chr3: 177012791 | 3q26.31 | Downstream | NAALADL2 | 0.00479 | 0.03097 | 0.30670 | ″ |
| rs6791089 | chr3: 176998117 | 3q26.31 | Intron | NAALADL2 | 0.00522 | 0.03097 | 0.33420 | ″ |
| rs9536962 | chr13: 54576937 | 13q21.1 | CNV | | 0.00569 | 0.03097 | 0.36380 | ″ |
| rs2250595 | chr12: 45642394 | 12q13.11 | — | | 0.00581 | 0.03097 | 0.37160 | ″ |
| rs4894734 | chr3: 177013204 | 3q26.31 | Downstream | NAALADL2 | 0.00749 | 0.03416 | 0.47940 | ″ |
| rs1481513 | chr8: 79171163 | 8q21.12 | CNV | | 0.00791 | 0.03416 | 0.50640 | ″ |
| rs7337921 | chr13: 23381053 | 13q12.12 | Intron | FLJ46358 | 0.00801 | 0.03416 | 0.51240 | ″ |
| rs1863080 | chr2: 36379940 | 2p22.3 | CNV | | 0.00937 | 0.03651 | 0.59960 | ″ |
| rs7944323 | chr11: 78903676 | 11q14.1 | CNV | | 0.00970 | 0.03651 | 0.62060 | ″ |
| rs10987251 | chr9: 128142815 | 9q33.3 | Intron | C9orf28 | 0.01512 | 0.05076 | 0.96790 | ″ |
| | | | (boundary) | | | | | |
| rs6974649 | chr7: 130463637 | 7q32.3 | CNV | | 0.01564 | 0.05076 | 1.00000 | ″ |
| rs4894733 | chr3: 177013074 | 3q26.31 | Downstream | NAALADL2 | 0.01658 | 0.05076 | 1.00000 | ″ |
| rs9508456 | chr13: 28914362 | 13q12.3 | Intron | KIAA0774 | 0.01724 | 0.05076 | 1.00000 | ″ |
| rs1796045 | chr2: 96986737 | 2q11.2 | CNV | | 0.01745 | 0.05076 | 1.00000 | ″ |
| rs11229410 | chr11: 57926867 | 11q12.1 | Coding exon | OR5B3 | 0.01908 | 0.05292 | 1.00000 | ″ |
| rs4745257 | chr9: 75582705 | 9q21.13 | CNV | | 0.02022 | 0.05292 | 1.00000 | ″ |
| rs12606567 | chr18: 48408860 | 18q21.2 | Intron | DCC | 0.02114 | 0.05292 | 1.00000 | ″ |
| rs2044747 | chr14: 42393956 | 14q21.2 | CNV | | 0.02150 | 0.05292 | 1.00000 | ″ |
| rs4646421 | chr15: 72803245 | 15q24.1 | Intron | CYP1A1 | 0.02509 | 0.05948 | 1.00000 | ″ |
| rs1440423 | chr5: 34425616 | 5p13.2 | CNV | | 0.02694 | 0.06002 | 1.00000 | ″ |
| rs1888156 | chr9: 128142661 | 9q33.3 | Coding exon | C9orf28 | 0.02720 | 0.06002 | 1.00000 | ″ |
| rs9941626 | chr2: 212059912 | 2q34 | Intron | ERBB4 | 0.03374 | 0.07197 | 1.00000 | ″ |
| rs2078520 | chr2: 80246156 | 2p12 | Intron | CTNNA2 | 0.03756 | 0.07755 | 1.00000 | ″ |
| rs1796028 | chr2: 96982680 | 2q11.2 | CNV | | 0.04168 | 0.08337 | 1.00000 | ″ |
| rs1482930 | chr15: 79651018 | 15q25.2 | CNV | | 0.04879 | 0.09461 | 1.00000 | ″ |
| rs11627027 | chr14: 93440170 | 14q32.13 | — | | 0.05285 | 0.09948 | 1.00000 | ″ |

TABLE 5
Insistence on sameness and rituals QTL associated with ASD subtypes

| SNP | SNP Position | Band | Location | Gene | UNADJ | FDR_BH | BONF | Subtype |
|---|---|---|---|---|---|---|---|---|
| rs1827924 | chr2: 228377979 | 2q36.3 | Promoter | CCL20 | 0.00016 | 0.00526 | 0.00526 | Moderate |
| rs17738966 | chr14: 54371969 | 14q22.2 | Downstream | GCH1 | 0.00053 | 0.00708 | 0.01711 | ″ |
| rs7950390 | chr11: 4587928 | 11p15.4 | Promoter | TRIM68 | 0.00066 | 0.00708 | 0.02124 | ″ |
| rs3861787 | chr11: 4604346 | 11p15.4 | CNV | | 0.00120 | 0.00784 | 0.03851 | ″ |
| rs317985 | chr5: 66773558 | 5q13.1 | CNV | | 0.00122 | 0.00784 | 0.03917 | ″ |
| rs3804967 | chr3: 7479914 | 3p26.1 | Intron | GRM7 | 0.00223 | 0.01187 | 0.07121 | ″ |
| rs3804968 | chr3: 7477700 | 3p26.1 | Intron | GRM7 | 0.00334 | 0.01313 | 0.10690 | ″ |
| rs13096022 | chr3: 7465129 | 3p26.1 | Intron | GRM7 | 0.00344 | 0.01313 | 0.11020 | ″ |
| rs6782718 | chr3: 7462776 | 3p26.1 | Intron | GRM7 | 0.00369 | 0.01313 | 0.11820 | ″ |
| rs9568011 | chr13: 47606812 | 13q14.2 | — | | 0.00474 | 0.01518 | 0.15180 | ″ |
| rs164187 | chr1: 160615261 | 1q23.3 | Promoter | C1orf111 | 0.00704 | 0.02046 | 0.22510 | ″ |
| rs10781238 | chr9: 76384432 | 9q21.13 | Intron | RORB | 0.01007 | 0.02402 | 0.32210 | ″ |
| rs9634811 | chr13: 47362176 | 13q14.2 | — | | 0.01032 | 0.02402 | 0.33030 | ″ |
| rs2469183 | chr15: 84992924 | 15q25.3 | CNV | | 0.01051 | 0.02402 | 0.33630 | ″ |
| rs317985 | chr5: 66773558 | 5q13.1 | CNV | | 0.00227 | 0.05839 | 0.07266 | Mild |
| rs1827924 | chr2: 228377979 | 2q36.3 | Promoter | CCL20 | 0.00365 | 0.05839 | 0.11680 | ″ |
| rs2574852 | chr17: 73003467 | 17q25.3 | Intron | SEPT9 | 0.00571 | 0.06085 | 0.18260 | ″ |

TABLE 6
Social development QTL associated with ASD subtype

| SNP | SNP Position | Band | Location | Gene | UNADJ P | FDR_BH | BONF | Subtype |
|---|---|---|---|---|---|---|---|---|
| rs12266938 | chr10: 3852940 | 10p15.1 | CNV | | 0.00005 | 0.00234 | 0.00234 | Mild |
| rs10519124 | chr2: 67819501 | 2p14 | — | | 0.00018 | 0.00403 | 0.00807 | ″ |
| rs2297172 | chr9: 71563166 | 9q21.11 | Intron | PTAR1 | 0.00056 | 0.00816 | 0.02447 | ″ |
| rs2255313 | chr12: 102773924 | 12q23.3 | CNV | | 0.00139 | 0.01500 | 0.06129 | ″ |
| rs6698676 | chr1: 84528583 | 1p31.1 | Promoter | SAMD13 | 0.00173 | 0.01500 | 0.07595 | ″ |
| rs2519866 | chr17: 27859883 | 17q11.2 | Intron | MYO1D | 0.00205 | 0.01500 | 0.08997 | ″ |
| rs10305860 | chr4: 148625337 | 4q31.23 | Intron | EDNRA | 0.00279 | 0.01756 | 0.12290 | ″ |
| rsl3205238 | chr6: 413597 | 6p25.3 | CNV | | 0.00331 | 0.01819 | 0.14560 | ″ |
| rs4873815 | chr8: 144796206 | 8q24.3 | Promoter | ZNF623 | 0.00417 | 0.01824 | 0.18360 | ″ |
| rs4832481 | chr2: 16986218 | 2p24.3 | CNV | | 0.00445 | 0.01824 | 0.19590 | ″ |
| rs30746 | chr5: 135366157 | 5q31.1 | CNV | | 0.00456 | 0.01824 | 0.20060 | ″ |
| rs10997162 | chr10: 67906707 | 10q21.3 | Intron | CTNNA3 | 0.00536 | 0.01964 | 0.23570 | ″ |
| rs3809282 | chr12: 110192180 | 12q24.11 | Intron | CUTL2 | 0.00606 | 0.02049 | 0.26640 | ″ |
| rs12115722 | chr9: 133418328 | 9q34.13 | CNV | | 0.00693 | 0.02178 | 0.30490 | ″ |
| rs10874468 | chr2: 96959718 | 2q11.2 | CNV | | 0.00991 | 0.02772 | 0.43610 | ″ |
| rs4809918 | chr20: 50734607 | 20q13.2 | — | | 0.01055 | 0.02772 | 0.46420 | ″ |
| rs12962411 | chr18: 27645521 | 18q12.1 | CNV | | 0.01071 | 0.02772 | 0.47120 | ″ |
| rs4959923 | chr6: 412773 | 6p25.3 | CNV | | 0.01291 | 0.03155 | 0.56790 | ″ |
| rs721087 | chr2: 4942340 | 2p25.2 | CNV | | 0.01662 | 0.03849 | 0.73120 | ″ |
| rs9479482 | chr6: 150399705 | 6q25.1 | — | | 0.01804 | 0.03968 | 0.79370 | ″ |
| rs10788819 | chr1: 150161717 | 1q21.3 | — | | 0.01975 | 0.04138 | 0.86900 | ″ |
| rs4646421 | chr15: 72803245 | 15q24.1 | Intron | CYP1A1 | 0.02509 | 0.05019 | 1.00000 | ″ |
| rs11138895 | chr9: 82693023 | 9q21.31 | CNV | | 0.02649 | 0.05067 | 1.00000 | ″ |
| rs11138885 | chr9: 82678684 | 9q21.31 | — | | 0.02998 | 0.05496 | 1.00000 | ″ |
| rs4905110 | chr14: 93432083 | 14q32.13 | — | | 0.03294 | 0.05703 | 1.00000 | ″ |
| rs2627468 | chr8: 3812607 | 8p23.2 | Intron | CSMD1 | 0.03494 | 0.05703 | 1.00000 | ″ |
| rs17192980 | chr5: 7030768 | 5p15.31 | CNV | | 0.03499 | 0.05703 | 1.00000 | ″ |
| rs10886048 | chr10: 118928872 | 10q25.3 | CNV | | 0.04260 | 0.06602 | 1.00000 | ″ |
| rs4811895 | chr20: 55639715 | 20q13.31 | — | | 0.04351 | 0.06602 | 1.00000 | ″ |
| rs13384439 | chr2: 187500106 | 2q32.1 | CNV | | 0.04620 | 0.06776 | 1.00000 | ″ |
| rs4416176 | chr2: 26536150 | 2p23.3 | Intron | OTOF | 0.05067 | 0.07081 | 1.00000 | ″ |
| rs11627027 | chr14: 93440170 | 14q32.13 | — | | 0.05285 | 0.07081 | 1.00000 | ″ |
| rs12183587 | chr6: 150396301 | 6q25.1 | — | | 0.05311 | 0.07081 | 1.00000 | ″ |
| rs2151206 | chr9: 14844072 | 9p22.3 | Intron | FREM1 | 0.05619 | 0.07272 | 1.00000 | ″ |
| rs6022029 | chr20: 50708572 | 20q13.2 | CNV | | 0.06062 | 0.07621 | 1.00000 | ″ |
| rs1996893 | chr9: 14880268 | 9p22.3 | Intron | FREM1 | 0.07259 | 0.08872 | 1.00000 | ″ |

TABLE 7
Highly significant SNPs across ASD subtypes

| CHR | SNP | BP | A1 | F_A | F_U | A2 | CHISQ | OR | Band |
|---|---|---|---|---|---|---|---|---|---|
| 5 | rs2277049 | 147883281 | A | 0.1153 | 0.0811 | C | 13.71 | 1.48 | 5q33.1 |
| 9 | rs757099 | 25727567 | C | 0.3909 | 0.4470 | T | 12.94 | 0.79 | 9p21.2 |
| 7 | rs7785107 | 10247968 | T | 0.0313 | 0.0560 | G | 12.79 | 0.54 | 7p21.3 |
| 5 | rs7725785 | 147882896 | A | 0.1144 | 0.0825 | C | 12.71 | 1.44 | 5q33.1 |
| 5 | rs2287581 | 31341022 | A | 0.0783 | 0.0531 | G | 11.62 | 1.51 | 5p13.3 |
| 9 | rs1231339 | 25730863 | A | 0.5341 | 0.4803 | C | 11.54 | 1.24 | 9p21.2 |
| 22 | rs2180055 | 47722427 | T | 0.1455 | 0.1131 | C | 10.1 | 1.34 | 22q13.32 |
| 19 | rs11671930 | 8023308 | C | 0.1346 | 0.1595 | T | 4.809 | 0.82 | 19p13.2 |
| 7 | rs7785107 | 10247968 | T | 0.0826 | 0.0560 | G | 10.01 | 1.52 | 7p21.3 |
| 11 | rs7950390 | 4587928 | G | 0.1067 | 0.0798 | T | 7.51 | 1.38 | 11p15.4 |
| 10 | rs12266938 | 3852940 | C | 0.2364 | 0.1983 | T | 7.14 | 1.25 | 10p15.1 |
| 11 | rs3861787 | 4604346 | T | 0.1015 | 0.0759 | G | 7.081 | 1.38 | 11p15.4 |
| 2 | rs1827924 | 228377979 | G | 0.2562 | 0.3262 | A | 14.2 | 0.71 | 2q36.3 |
| 14 | rs17738966 | 54371969 | A | 0.1309 | 0.0902 | G | 11.99 | 1.52 | 14q22.2 |
| 11 | rs7950390 | 4587928 | G | 0.0441 | 0.0798 | T | 11.59 | 0.53 | 11p15.4 |
| 11 | rs3861787 | 4604346 | T | 0.0427 | 0.0759 | G | 10.49 | 0.54 | 11p15.4 |
| 5 | rs317985 | 66773558 | A | 0.3208 | 0.2635 | G | 10.45 | 1.32 | 5q13.1 |
| 5 | rs7725785 | 147882896 | A | 0.0580 | 0.0825 | C | 5.168 | 0.69 | 5q33.1 |
| 10 | rs12266938 | 3852940 | C | 0.1370 | 0.1983 | T | 16.33 | 0.64 | 10p15.1 |
| 16 | rs730168 | 73707776 | A | 0.1731 | 0.2344 | G | 14.34 | 0.68 | 16q23.1 |
| 2 | rs10519124 | 67819501 | A | 0.1873 | 0.1366 | G | 13.99 | 1.46 | 2p14 |
| 10 | rs6482516 | 18879356 | A | 0.3101 | 0.2471 | G | 13.91 | 1.37 | 10p12.33 |
| 19 | rs11671930 | 8023308 | C | 0.2119 | 0.1595 | T | 13.21 | 1.42 | 19p13.2 |
| 9 | rs2297172 | 71563166 | C | 0.0932 | 0.1388 | T | 11.92 | 0.64 | 9q21.11 |
| 5 | rs317985 | 66773558 | A | 0.2119 | 0.2635 | G | 9.317 | 0.75 | 5q13.1 |
| 2 | rs1827924 | 228377979 | G | 0.3796 | 0.3262 | A | 8.45 | 1.26 | 2q36.3 |
| 9 | rs1231339 | 25730863 | A | 0.4355 | 0.4803 | C | 5.283 | 0.83 | 9p21.2 |
| 9 | rs757099 | 25727567 | C | 0.4858 | 0.4470 | T | 4.051 | 1.17 | 9p21.2 |
| 5 | rs7725785 | 147882896 | A | 0.0620 | 0.0825 | C | 3.814 | 0.74 | 5q33.1 |

| | | | | | | Subtype |
|---|---|---|---|---|---|---|
| CHR | Location | Gene | UNADJ | FDR_BH | BONF | (#cases) |
| 5 | Intron | HTR4 | 0.00021 | 0.00173 | 0.00405 | Language (639) |
| 9 | CNV | | 0.00032 | 0.00173 | 0.00611 | ″ |
| 7 | CNV | | 0.00035 | 0.00173 | 0.00663 | ″ |
| 5 | Intron | HTR4 | 0.00036 | 0.00173 | 0.00690 | ″ |
| | (boundary) | | | | | |
| 5 | Intron | CDH6 | 0.00065 | 0.00215 | 0.01242 | ″ |
| | (boundary) | | | | | |
| 9 | CNV | | 0.00068 | 0.00215 | 0.01293 | ″ |
| 22 | CNV | | 0.00148 | 0.00402 | 0.02811 | ″ |
| 19 | Promoter | CCL25 | 0.02831 | 0.05976 | 0.53790 | ″ |
| 7 | CNV | | 0.00156 | 0.02962 | 0.02962 | Intermed (478) |
| 11 | Promoter | TRIM68 | 0.00614 | 0.03700 | 0.11660 | ″ |
| 10 | CNV | | 0.00754 | 0.03700 | 0.14320 | ″ |
| 11 | CNV | | 0.00779 | 0.03700 | 0.14800 | ″ |
| 2 | Promoter | CCL20 | 0.00016 | 0.00312 | 0.00312 | Moderate (363) |
| 14 | Downstream | GCH1 | 0.00053 | 0.00420 | 0.01016 | ″ |
| 11 | Promoter | TRIM68 | 0.00066 | 0.00420 | 0.01261 | ″ |
| 11 | CNV | | 0.00120 | 0.00465 | 0.02287 | ″ |
| 5 | CNV | | 0.00122 | 0.00465 | 0.02326 | ″ |
| 5 | Intron | HTR4 | 0.02301 | 0.07286 | 0.43720 | ″ |
| | (boundary) | | | | | |
| 10 | CNV | | 0.00005 | 0.00091 | 0.00101 | Mild (387) |
| 16 | Intron | LDHD | 0.00015 | 0.00091 | 0.00290 | ″ |
| 2 | | | 0.00018 | 0.00091 | 0.00348 | ″ |
| 10 | Intron | NSUN6 | 0.00019 | 0.00091 | 0.00365 | ″ |
| 19 | Promoter | CCL25 | 0.00028 | 0.00106 | 0.00530 | ″ |
| 9 | Intron | PTAR1 | 0.00056 | 0.00176 | 0.01057 | ″ |
| 5 | CNV | | 0.00227 | 0.00616 | 0.04314 | ″ |
| 2 | Promoter | CCL20 | 0.00365 | 0.00867 | 0.06934 | ″ |
| 9 | CNV | | 0.02154 | 0.04547 | 0.40920 | ″ |
| 9 | CNV | | 0.04414 | 0.08386 | 0.83860 | ″ |
| 5 | Intron | HTR4 | 0.05082 | 0.08778 | 0.96550 | ″ |
| | (boundary) | | | | | |

TABLE 8

| | Association analysis of 6 SNPs from original GWA study (9) with ASD subtypes | | | | | | | | | | | |
|---|---|---|---|---|---|---|---|---|---|---|---|---|
| | | | | | | | | | | | | Subtype |
| CHR | SNP | BP | A1 | F_A | F_U | A2 | CHISQ | OR | UNADJ | FDR_BH | BONF | (# cases) |
| 5 | rs1896731 | 25934777 | C | 0.4146 | 0.3617 | T | 7.61 | 1.25 | 0.00580 | 0.02655 | 0.03483 | Moderate (363) |
| 5 | rs10038113 | 25938099 | C | 0.4711 | 0.4196 | T | 6.853 | 1.232 | 0.00885 | 0.02655 | 0.05310 | ″ |
| 5 | rs7704909 | 25934678 | C | 0.3154 | 0.3512 | T | 3.569 | 0.8512 | 0.05888 | 0.07402 | 0.35330 | ″ |
| 5 | rs4327572 | 26008578 | T | 0.314 | 0.3488 | C | 3.381 | 0.8547 | 0.06595 | 0.07402 | 0.39570 | ″ |
| 5 | rs12518194 | 25987318 | G | 0.3135 | 0.3475 | A | 3.225 | 0.8576 | 0.07250 | 0.07402 | 0.43500 | ″ |
| 5 | rs4307059 | 26003460 | C | 0.3115 | 0.3454 | T | 3.192 | 0.8574 | 0.07402 | 0.07402 | 0.44410 | ″ |
| | Comparison with Combined cases and other subtypes | | | | | | | | | | | |
| | | | | | | | | | | | | Subtype |
| CHR | SNP | BP | A1 | F_A | F_U | A2 | CHISQ | OR | UNADJ P | FDR_BH | BONF | (# cases) |
| 5 | rs4307059 | 26003460 | C | 0.3317 | 0.3454 | T | 1.741 | 0.9408 | 0.18700 | 0.36070 | 1.00000 | Combined cases (1867) |
| 5 | rs12518194 | 25987318 | G | 0.3341 | 0.3475 | A | 1.677 | 0.9423 | 0.19540 | 0.36070 | 1.00000 | ″ |
| 5 | rs4327572 | 26008578 | T | 0.336 | 0.3488 | C | 1.534 | 0.9448 | 0.21550 | 0.36070 | 1.00000 | ″ |
| 5 | rs7704909 | 25934678 | C | 0.339 | 0.3512 | T | 1.378 | 0.9477 | 0.24050 | 0.36070 | 1.00000 | ″ |
| 5 | rs10038113 | 25938099 | C | 0.4252 | 0.4196 | T | 0.278 | 1.024 | 0.59780 | 0.71730 | 1.00000 | ″ |
| 5 | rs1896731 | 25934777 | C | 0.365 | 0.3617 | T | 0.104 | 1.015 | 0.74760 | 0.74760 | 1.00000 | ″ |
| 5 | rs1896731 | 25934777 | C | 0.3404 | 0.3617 | T | 1.997 | 0.9108 | 0.15760 | 0.43880 | 0.94560 | Language-impaired (639) |
| 5 | rs10038113 | 25938099 | C | 0.402 | 0.4196 | T | 1.28 | 0.9301 | 0.25790 | 0.43880 | 1.00000 | ″ |
| 5 | rs4307059 | 26003460 | C | 0.3318 | 0.3454 | T | 0.828 | 0.941 | 0.36290 | 0.43880 | 1.00000 | ″ |
| 5 | rs4327572 | 26008578 | T | 0.3357 | 0.3488 | C | 0.771 | 0.9433 | 0.38000 | 0.43880 | 1.00000 | ″ |
| 5 | rs2518194 | 25987318 | G | 0.3349 | 0.3475 | A | 0.711 | 0.9455 | 0.39910 | 0.43880 | 1.00000 | ″ |
| 5 | rs7704909 | 25934678 | C | 0.3396 | 0.3512 | T | 0.6 | 0.95 | 0.43880 | 0.43880 | 1.00000 | ″ |
| 5 | rs7704909 | 25934678 | C | 0.3577 | 0.3512 | T | 0.15 | 1.029 | 0.69830 | 0.98300 | 1.00000 | Intermediate (478) |
| 5 | rs4327572 | 26008578 | T | 0.3512 | 0.3488 | C | 0.019 | 1.01 | 0.88950 | 0.98300 | 1.00000 | ″ |
| 5 | rs12518194 | 25987318 | G | 0.3462 | 0.3475 | A | 0.006 | 0.9944 | 0.94030 | 0.98300 | 1.00000 | ″ |
| 5 | rs10038113 | 25938099 | C | 0.4184 | 0.4196 | T | 0.004 | 0.9952 | 0.94690 | 0.98300 | 1.00000 | ″ |
| 5 | rs1896731 | 25934777 | C | 0.3609 | 0.3617 | T | 0.002 | 0.9966 | 0.96340 | 0.98300 | 1.00000 | ″ |
| 5 | rs4307059 | 26003460 | C | 0.345 | 0.3454 | T | 5E−04 | 0.9984 | 0.98300 | 0.98300 | 1.00000 | ″ |
| 5 | rs7704909 | 25934678 | C | 0.3372 | 0.3512 | T | 0.574 | 0.9399 | 0.44850 | 0.74860 | 1.00000 | Mild (387) |
| 5 | rs4307059 | 26003460 | C | 0.3342 | 0.3454 | T | 0.365 | 0.9514 | 0.54550 | 0.74860 | 1.00000 | ″ |
| 5 | rs4327572 | 26008578 | T | 0.3385 | 0.3488 | C | 0.313 | 0.9553 | 0.57590 | 0.74860 | 1.00000 | ″ |
| 5 | rs12518194 | 25987318 | G | 0.3372 | 0.3475 | A | 0.312 | 0.9553 | 0.57630 | 0.74860 | 1.00000 | ″ |
| 5 | rs10038113 | 25938099 | C | 0.4289 | 0.4196 | T | 0.241 | 1.039 | 0.62390 | 0.74860 | 1.00000 | ″ |
| 5 | rs1896731 | 25934777 | C | 0.3643 | 0.3617 | T | 0.021 | 1.012 | 0.88530 | 0.88530 | 1.00000 | ″ |

TABLE 9
List of behavioral categories and associated ADI-R items
used for quantitative trait (QT) analyses.

| | | | | Sensory |
|---|---|---|---|---|
| Language | Nonverbal | | Social | issues & |
| deficits | communication | Play skills | development | stereotypies |
| CARTIC | CCOMPSL | CPLAY | GAZE5 | CNOISE |
| ARTICF5 | COMPSL5 | PLAY5 | CSSMILE | ENOISE |
| CSTEREO | CUSEBOD | CPEERPL | SSMILE5 | CABINR |
| ESTEREO | EUSEBOD | PEERPL5 | CSHOW | EABINR |
| CCHAT | CPOINT | CSOPLAY | SHOW5 | CHFMAN |
| CHAT5 | POINT5 | SOPLAY5 | COSHARE | EHFMAN |
| CCONVER | CNOD | CINTCH | OSHARE5 | COTHMAN |
| CONVER5 | NOD5 | INTCH5 | CSHARE | EOTHMAN |
| CINAPPQ | CHSHAKE | CRESPCH | SHARE5 | CMLHAND |
| EINAPPQ | HSHAKE5 | RESPCH5 | COCOMF | EMLHAND |
| CPRON | CINSGES | CGRPLAY | OCOMF5 | CGAIT |
| EPRON | INSGES5 | GRPLAY5 | CQUALOV | GAIT5 |
| CNEOID | AVOICE5 | CFRIEND | QUALOV5 | CHVENT |
| ENEOID | CIMIT | FREND15 | CRFACEX | EHVENT |
| CVERRIT | IMIT5 | | RFACEX5 | CFAINT |
| EVERRIT | | | CINAPFE | EFAINT |
| CINR | | | EINAPFE | |
| EINR | | | CQRESP | |
| CSPEECH | | | QRESP5 | |
| SPEECH5 | | | CINITIA | |
| | | | INITIA5 | |
| | | | CSOCDIS | |
| | | | SOCDIS5 | |

TABLE 10
Nucleotide sequences from the National Center for Biotechnology
Information (NCBI) Database of Short Genetic Variations (dbSNP)
of the SNPs disclosed herein. The single nucleotide polymorphism
between the major and minor alleles is bracketted.

rs12407665 [Homo sapiens] (SEQ ID NO: 1)
TAGAACCAGCACACATTGGCCAAATA[C/G/T]GGCCCATGGCTCTCGAATGGTCTTT
rs17828521 [Homo sapiens] (SEQ ID NO: 2)
ACCTACAATCAAATTGTTGTCTTCTC[C/T]TTACTGATCTTTGAAACACCTTTAA
rs9474831 [Homo sapiens] (SEQ ID NO: 3)
AACTCTCACACACATTGACGCTGTTT[C/T]CTTCTCAGCTATTCAAAGTCCATTT
rs6454792 [Homo sapiens] (SEQ ID NO: 4)
TATCACAGATGTACTGTGCTGATAGA[A/G]AAGTCTGAGCTATTGGATTTGCCAG
rs10183984 [Homo sapiens] (SEQ ID NO: 5)
GCTGTTGTTGAATAAATATACTATAC[A/G]TGTTAATCAGATCCATTAGATTGAT
rs11969265 [Homo sapiens] (SEQ ID NO: 6)
AGTATAATGTGAGATGATGCTGCATA[C/T]AGCCATGGTCAGAAACGGGGAAAAG
rs1231339 [Homo sapiens] (SEQ ID NO: 7)
TCTAACAAAATGTCTTAGGCTGGGTA[A/C]TTTATAAGGCTAAGACATTTACTTC
rs10806416 [Homo sapiens] (SEQ ID NO: 8)
AGATGGCTGTGCTGGCACAGAGCATG[C/T]CCCTCCAGTTCTCCAAGATGGAGCA
rs7785107 [Homo sapiens] (SEQ ID NO: 9)
TCCTATAGCTTTAGCTCTAAATCAGT[G/T]AATTCCAATTTTTTATATTATACTT
rs2277049 [Homo sapiens] (SEQ ID NO: 10)
TCTCCTCTTTTTACCCTGTATTCTTT[A/C]TTTTATTTCCTTATTTTTATTCTCT
rs757099 [Homo sapiens] (SEQ ID NO: 11)
TCTCTTGTGAATTGTAGTAATGGAAG[C/T]ACTACTTTAAAACTTCTTCAGCAGA
rs7725785 [Homo sapiens] (SEQ ID NO: 12)
CTAATATTATTTATTCATTTAGGAAC[A/C]CCATGCAAAGTTGATCAGACAGTAA
rs758158 [Homo sapiens] (SEQ ID NO: 13)
TCCCTGGGGAGGGAGATCTGAGCTCC[A/G]GGTTAGAAGTTTGGTAGAGGTTAGC
rs2287581 [Homo sapiens] (SEQ ID NO: 14)
AAGTCCAAGAGCTTGAGGTTGGGGGT[A/G]GGGGAAGAAGTGACATATTTAAAGC
rs17830215 [Homo sapiens] (SEQ ID NO: 15)
GTGTTTTTTAAAGTGATTTCCTGGCA[C/T]CCTACAAACAGTAGCATTTTAATCC
rs2180055 [Homo sapiens] (SEQ ID NO: 16)
ACATGGTTGGAGTCCTTCAGAGGGTC[C/T]AGCTCACCAGAGGACGCCCAGAGTC
rs12893752 [Homo sapiens] (SEQ ID NO: 17)
gttatccccagggagggTTGCTAGCA[C/T]GTTGTCACCTTTCAATATGGCAGAT
rs13205238 [Homo sapiens] (SEQ ID NO: 18)
TACATCAGCTCAGCTGGAACAGACCC[A/G]TCCAAAGAGGAGAATTTTTGTTTTG
rs11671930 [Homo sapiens] (SEQ ID NO: 19)
GGGGACCCACCAGGGAACAGGTGGCT[C/T]GAGGGCGGGGGGAGCATGAAGGTAA
rs11229410 [Homo sapiens] (SEQ ID NO: 20)
AAAGATATTGAAGCTCACAACATAAA[A/C/T]AAGAACAAGCTCGCTAATATGTCTA
rs11229413 [Homo sapiens] (SEQ ID NO: 21)
CATGGGATTGTGGAGACAGGAATCCC[A/G]GAATATCAATACAATAATTCCCAGG
rs11229411 [Homo sapiens] (SEQ ID NO: 22)
ATCAGAGCAAGAGAGAACCATGACTG[C/T]TGGAATATCACAGAAAAAGTGATGG
rs11721070 [Homo sapiens] (SEQ ID NO: 23)
AAAAGTACCGCACATCAAAACAGTAG[C/T]TGTTTTCTTCAACCCACACGCGCAC
rs12466917 [Homo sapiens] (SEQ ID NO: 24)
CTTATAAAGAGCCAGATCATAAATAC[A/G]TAGGGTTTGTGGGCCATATGGTCTC
rs13076171 [Homo sapiens] (SEQ ID NO: 25)
TTTGAAATACCTGGAGTGGTTCCACT[A/G]TGACAACCAGATCTTGACTCTTACA
rs7930778 [Homo sapiens] (SEQ ID NO: 26)
TATTAAATAAGGTGGAAAAGACGTAA[C/T]GTGTGGCCTTGTTTCAACGTGCCAA
rs12279895 [Homo sapiens] (SEQ ID NO: 27)
TCTCAACAACTTTCTTGAATGCACTC[C/T]TCACTTCCTTGTTCCTCAGACTATA
rs730168 [Homo sapiens] (SEQ ID NO: 28)
TGTGGAAGAAGGCCCTGAAGGAAAGG[A/G]CCTGGGTTCCAGGCCAGGCTCTGTC
rs13021324 [Homo sapiens] (SEQ ID NO: 29)
CTTCAAACAAGACTTCCAAGACCAAA[C/T]CAAATTCTCAGGGATCATTTTCTTC
rs564127 [Homo sapiens] (SEQ ID NO: 30)
TCTCAAGGAAGAATATAAATAAGTCA[C/T]TGACTATCATTTGCACTCTGATCTC
rs393076 [Homo sapiens] (SEQ ID NO: 31)
TTGGAACCATAGCAGGATCTGATAGT[A/G]ACCCTGAAGCTGGAAGGAACCTTGG
rs1938651 [Homo sapiens] (SEQ ID NO: 32)
GAGCTTGGGCTCTGGGAAGAGGTGCA[C/T]GTCATTTCTACATGTACAACCTAAG
rs1938672 [Homo sapiens] (SEQ ID NO: 33)
GCATCATCCAGCCTAGGGTTTTACTA[C/T]CATCTTAGGGAGAGCAGCACGGCAT
rs4804202 [Homo sapiens] (SEQ ID NO: 34)
CAGACAGAGAAGCAGCAGCAAATCAG[C/T]GGAGGCCAGGATTGATAGCTTCCCC
rs665036 [Homo sapiens] (SEQ ID NO: 35)
CTTGAGATTGTGTTGGTGTTAATATA[C/T]ATAGCCTCACTTTGAGGGCAGGTGA
rs4527692 [Homo sapiens] (SEQ ID NO: 36)
GAAGAATCAATAAGTCGCTTTTGGCT[A/C/G/T]TAAAATGGCTCCTGAGCAGTCACCT
rs519514 [Homo sapiens] (SEQ ID NO: 37)
CTAGGACTAAAGGCAATATAGACTAC[C/T]GTGATACTATCTAGTTCGCGAAAGT
rs3133855 [Homo sapiens] (SEQ ID NO: 38)
ACACTTTTGCATCCACATGGTGTCTC[A/G]ACACAGCTAAGTCCTCAGTCATAAC
rs1938670 [Homo sapiens] (SEQ ID NO: 39)
ACATAGAGGTGACACACAGGGCTGAA[A/G]GTGTGGGTGGGTTTTCAAGTTGGCA
rs13205238 [Homo sapiens] (SEQ ID NO: 18)
TACATCAGCTCAGCTGGAACAGACCC[A/G]TCCAAAGAGGAGAATTTTTGTTTTG
rs1996893 [Homo sapiens] (SEQ ID NO: 40)
AGACATACCTTTGCCTACAACACAAA[C/T]TCATTAGGTTTCCTTCCTTAGATTT
rs12606567 [Homo sapiens] (SEQ ID NO: 41)
GCAAATATGTGCCTGTTACAAACTTG[C/T]CTTCATCTGTGTGTACAGCCATTCA
rs3769845 [Homo sapiens] (SEQ ID NO: 42)
AAGCCTGAGTTCCAGCCTTTGCTGTT[C/T]CAGGAGTGACTGTTCCATTCTGAGT
rs2422675 [Homo sapiens] (SEQ ID NO: 43)
AGGTAAAAAACACAACAGATGCCAAT[A/G]GCAAGAGTGTCTAGATATTGAAATG
rs4798405 [Homo sapiens] (SEQ ID NO: 44)
AAGTCCCATAAATTCCATTTCTTGAA[A/G]GAAAAGGCCATGAGTCAGTATTTGA
rs10040891 [Homo sapiens] (SEQ ID NO: 45)
TTAGTTTCCGTTATTTACGTGTGTGA[C/T]TCATGGAAATGTTTATGTCTTGCCC
rs8181738 [Homo sapiens] (SEQ ID NO: 46)
TTAAGAATGGGTTTTCAAACAAACTT[G/T]CTAGTCTGTTTTTGAAAAGTCAAGG
rs11950809 [Homo sapiens] (SEQ ID NO: 47)
AAGACACATGTCACACACATGGCACG[A/C]TCAACAATGTCAGTCTAGTCATAGG
rs1930 [Homo sapiens] (SEQ ID NO: 48)
GATGACACTGATGGTGACGATACTGA[C/T]GATAGTAATAACACTTACTGAATAC
rs4894734 [Homo sapiens] (SEQ ID NO: 49)
TCCAGCTGGCACAAGGGTCTGTGGAA[A/C]GTGCATACGTGTTCCCAGCTCTAA
rs1482930 [Homo sapiens] (SEQ ID NO: 50)
TCCTTTCTAAGAAGATCCTTCAGCCT[A/G]TATTCTGGGCATACTTTCTCACAAC
rs11671930 [Homo sapiens] (SEQ ID NO: 19)
GGGGACCCACCAGGGAACAGGTGGCT[C/T]GAGGGCGGGGGGAGCATGAAGGTAA
rs4980777 [Homo sapiens] (SEQ ID NO: 51)
GATGAATCTGATCAATGATGATGAAT[A/G]GACTATGTTACACATAACGTCATAG
rs1481513 [Homo sapiens] (SEQ ID NO: 52)
AACAGCTCCCCATACCCAACTATCTA[C/T]CCTAAAATGACATGCCACTAGTGAA
rs10987251 [Homo sapiens] (SEQ ID NO: 53)
TGTGTCCAAGACCTTCTGGCCCTTGC[A/G]TGTAAGATGTGCTTCTTCCCTCTAG
rs2151206 [Homo sapiens] (SEQ ID NO: 54)
AGCTCTCTTGGCTCCTTCCTATGTAT[A/C/G/T]ATGATAACTGATCCAATTGATCCTT
rs2044747 [Homo sapiens] (SEQ ID NO: 55)
TGAAGACCTAAGTATTGACTAGTTGT[G/T]TATGTTGGACCACATTTTAATTTCA
rs1440423 [Homo sapiens] (SEQ ID NO: 56)
CACTGTTGGTCTCCATGTTGTCAAAG[C/T]ATAGAGCAATGAGAGTTTTTGACCA
rs4745257 [Homo sapiens] (SEQ ID NO: 57)
TCCAAGGGAGAGGTCACAGGTCCTCA[A/C]CTTTTGAGCAGAGGAGTGTCTGACA
rs2779499 [Homo sapiens] (SEQ ID NO: 58)
AGGGCTCACCAGTTTGAGAACTGCAG[C/T]AGCCTTCGACAGCCTTCCTGAATCA
rs1796028 [Homo sapiens] (SEQ ID NO: 59)
CTGCTGGCATGCTCCATTCTATCCAC[A/G]TGCCCGGTCACATGGAGACTTTCAG
rs1888156 [Homo sapiens] (SEQ ID NO: 60)
GACCTCTCAGAAGCCTTGCCAGAAAC[A/G]TCAATGGATCCCATCACGGGAGTCG
rs6734788 [Homo sapiens] (SEQ ID NO: 61)
CTTATCTTGCCATAGCTTTAGGATAT[C/T]TGAAGAATGTGTTCATAGAAAATGA
rs7605424 [Homo sapiens] (SEQ ID NO: 62)
AAAACACTGTTTAACATCTGAAGTTC[A/G]TTTGCAAGAAGAGTAGATGAGCTAG
rs4627775 [Homo sapiens] (SEQ ID NO: 63)
AAGTCTGCGGGAGAAATGGCATTAAC[G/T]GGCAATAAATGGGACTGACAGAAAT
rs5009527 [Homo sapiens] (SEQ ID NO: 64)
CGGACATCTGCCGGTTGGTGGTAAAG[C/T]TGTTGATTTTAGGAAATTCTAGAGA
rs1796045 [Homo sapiens] (SEQ ID NO: 65)
CTTTCATTTCCTCCTTGTGCCAAGGA[C/T]TTCAAGGCCAGACATAAGAGTGGGA
rs1863080 [Homo sapiens] (SEQ ID NO: 66)
CCCAAATAAATCTTTAAAGCCAAAAA[A/C]CAGATTTACATGTGTGTCCTGTGTT
rs7337921 [Homo sapiens] (SEQ ID NO: 67)
CTGTGCCTGTTTATTTCACGGATTTC[A/G]GGTTAACCATTACAGAAAGGCCATG
rs6452136 [Homo sapiens] (SEQ ID NO: 68)
AATTTCCCATTGACCCAAAATCATTG[C/T]GGGGCAATTCAAATTTAACAGGTGG
rs2168709 [Homo sapiens] (SEQ ID NO: 69)
ACTGCTAACAAATAGCAGTGTTTTGA[A/C/G/T]TTTCCTGTTCTTTCTACCTCTTCAA
rs4386512 [Homo sapiens] (SEQ ID NO: 70)
CCCTGCATTACACTAGTCATTATATC[C/T]ATTTCAAGCAAAAGGGATTTTAAAA
rs12614870 [Homo sapiens] (SEQ ID NO: 71)
TGGTAGTTGTTTGGCATAAACACAAC[A/G]GTCTAAATGGATGGTGACAGGCAAC
rs10491885 [Homo sapiens] (SEQ ID NO: 72)
TTTCTGTCTGGTTAAGTGAAGACGAA[A/C/G/T]GAATGAAGAATCACAGTGTTCTTAC
rs4646421 [Homo sapiens] (SEQ ID NO: 73)
ATCTGACCACTCTTCAAAAGGAGGTA[A/C/G/T]ATGTGACAGCAGCTGGAAATTTCCA
rs4894733 [Homo sapiens] (SEQ ID NO: 74)
TCAGAAGCTTGGGAGTCTCCGCTCCC[G/T]CAGACTCCCATACCAGAAGCAGGAA
rs7944323 [Homo sapiens] (SEQ ID NO: 75)
GGACCTTGCTAGAGGACTTAAATGAG[G/T]GTGGAGCCACAAGATGGACAGAGCC
rs6791089 [Homo sapiens] (SEQ ID NO: 76)
TTTTGACTGGCTATTGGTCAGTTCCT[A/C]GTCTTTCCCAATCTGAAAAATGGGT
rs17770167 [Homo sapiens] (SEQ ID NO: 77)
AATGCTAGTAGCAGAGATTTTACTTT[A/G]AGCCAGAGTCAAATCCAATGTAGGG
rs6698676 [Homo sapiens] (SEQ ID NO: 78)
TTGATGCAATAATATCTGTTATGTAA[G/T]AGAAGCTCAACAAATTTTTATTTAT
rs11664663 [Homo sapiens] (SEQ ID NO: 79)
ATTCCTACTATTAGCAGAAATTGCAG[C/T]ATTCATACCCATAGCCAACCCTGGG
rs6482516 [Homo sapiens] (SEQ ID NO: 80)
AGAATGTACTCAAAGGCAGCTTTAGA[A/G]ACTAAATCATTATAGAACATCCATT
rs11082277 [Homo sapiens] (SEQ ID NO: 81)
GAGTCTGATCACTCGTTTACTGACAA[A/G]TAAAACATACCTGTCCCATGTCTCC
rs6988293 [Homo sapiens] (SEQ ID NO: 82)
TACTTTAAATAGTGTTTAAGAGCATA[A/G]GATTTAGAGCCTGAAACACCTGTGT
rs6974649 [Homo sapiens] (SEQ ID NO: 83)
TAATAGGGGTGATATGTGCTGAAAAG[A/C]TGAAAGTAGTGTAAGCATTTTTTGA
rs730168 [Homo sapiens] (SEQ ID NO: 28)
TGTGGAAGAAGGCCCTGAAGGAAAGG[A/G]CCTGGGTTCCAGGCCAGGCTCTGTC
rs1461710 [Homo sapiens] (SEQ ID NO: 84)
TTTTAGAACTGTCAACTCACTTCCAC[A/G]TGTATTGGGTGTCTATACCATTATT
rs9941626 [Homo sapiens] (SEQ ID NO: 85)
GTATAGAATATAACAGCTTCATCAGA[G/T]AATATATTCTTAAAATAATCTATTT
rs3745651 [Homo sapiens] (SEQ ID NO: 86)
ATATATTACCAGCCTTTTCTAACCCA[C/T]GAAAGGACTCACACTGGAGAGAAAC
rs9536962 [Homo sapiens] (SEQ ID NO: 87)
ATTGTTGGGCATGACTCAATTGAGAG[A/G]GAATAGACCCATGAGAATCAGATAC
rs7529505 [Homo sapiens] (SEQ ID NO: 88)
GCACTCCAGGGAAGTCTTTGGAATTA[C/T]AGCTATAGGAAAACAAGTAAAATGC
rs9342127 [Homo sapiens] (SEQ ID NO: 89)
CCTTGATCATTATCCTGAGTGACTTT[A/G]TCCTTAAAGTGACTATCTTTTTAAC
rs1554547 [Homo sapiens] (SEQ ID NO: 90)
TGGCTCTATCAAATACTGTTATATTC[A/G]TATTACTTGTGAATAACCTGAGGTC
rs9508456 [Homo sapiens] (SEQ ID NO: 91)
GAGGTTCCAAAATTCTCAATTCACAG[C/T]GACCACAGTGTCTCAGTAATTTTTT
rs2078520 [Homo sapiens] (SEQ ID NO: 92)
TGCAGGTATGAAAAAAATGCTTCAAA[A/G]AGCAATTACTAACACACTTGAAACA
rs9569991 [Homo sapiens] (SEQ ID NO: 93)
CCTAAGCCAGAGCCTGTCACATGATA[A/G]GTTCAAAATAAATATTGGTTTAATG
rs3825597 [Homo sapiens] (SEQ ID NO: 94)
TCTTCACTCTCTAGCACATACCTGAT[A/G]AGACTTAGCAGAAGTAAACTCCTTC
rs3754741 [Homo sapiens] (SEQ ID NO: 95)
ACCACCCATTCTAAGGATCACACTAT[C/T]AGCATATACTCGTTTTAATTAAAGA
rs2250595 [Homo sapiens] (SEQ ID NO: 96)
TATTGAATATTCAGCAGCTTCTCACA[A/G]TCTAGAGAACTATTGAAGGCTCTCC
rs1055518 [Homo sapiens] (SEQ ID NO: 97)
TTTGCATTGCTGCTTCTCTTCACCCC[A/G]TGGAGGCTATGTCACCCTAACTATC
rs2600685 [Homo sapiens] (SEQ ID NO: 98)
AGTATTTACTGTGATTCGTTGAATCC[A/G]TGAAGAACTAATCCAACTCTCCAGG
rs164187 [Homo sapiens] (SEQ ID NO: 99)
TCACTGAAAAGTTTGTGGGAGCCTGG[C/T]GGTTCCAGGGTGCACACCACTCCTT
rs3809854 [Homo sapiens] (SEQ ID NO: 100)
ATCACCTCCCTCACAGCCCAGCTCCA[C/T]GTTCACCCCCACCAGGAAGTCTTCC
rs3804967 [Homo sapiens] (SEQ ID NO: 101)
AAATTATATGTGCCAGAAATTTAACA[A/G]GAGTGCAGGGTTTATGCTGGAAAAG
rs3804968 [Homo sapiens] (SEQ ID NO: 102)
TAAGCAGGTAAAAGGTTTAGCAGTGT[A/G]TTTGTCAGGTATTCAGTAAATATTG
rs317985 [Homo sapiens] (SEQ ID NO: 103)
ATTAATGAAATTCCCAAGAGAAAGTC[A/G]TTGCAGAAAGTTTTAATCTTACAGA
rs9634811 [Homo sapiens] (SEQ ID NO: 104)
TACTATGTTATGTATATTTTACCACA[A/G]TACTAAGAAGAATAACTAATGCCCC
rs7819605 [Homo sapiens] (SEQ ID NO: 105)
CAGCCAGGAATCTAGGATGGTTAGAA[A/G]AAAAGATATTTCTCTCTAACATACC
rs7950390 [Homo sapiens] (SEQ ID NO: 106)
TAGGCTTGATGAGAATATAATCTTAG[G/T]CTTGAAGGCTTTAAAGGGGAAGAAA
rs4436186 [Homo sapiens] (SEQ ID NO: 107)
ATTGTAAAGGTAAAATATATCTTTAC[A/G]TTGGAGAGATCTGGTGGTCTCTACC
rs4838964 [Homo sapiens] (SEQ ID NO: 108)
TGGAAGAGTTTATGTGAGTTTGTACT[A/G]TTACTTTCTTCAGTGATTGATAGAA
rs1827924 [Homo sapiens] (SEQ ID NO: 109)
TGTCTTTATTAGCAGTATGAGAGCAG[A/G]CAAATACACCTACCTAAAATTAAGC
rs7699496 [Homo sapiens] (SEQ ID NO: 110)
CAGGGCAGATGAAGTCATAAAAGCTG[A/G]GCTCCCTGTGTTCTGAAGATGACCA
rs3861787 [Homo sapiens] (SEQ ID NO: 111)
GCACATTGCATATACGGAGAGCATAG[G/T]AGACATAATGACCATGGATAGAAAA
rs6782718 [Homo sapiens] (SEQ ID NO: 112)
TCCAAACATCAATATTTAAGTTAATT[A/G]TATGCTGTTACTACTCAGGACTTCA
rs11038286 [Homo sapiens] (SEQ ID NO: 113)
CTGCACTCACATCCCCGGGGATCACA[A/G]ATGCAAATACAGCTTATGCATGGGA
rs693442 [Homo sapiens] (SEQ ID NO: 114)
CACCCAAAGGTGAAGGTGCACTGGGA[A/G]AAAATGAAGTTGCGTGGGGGCATCA
rs1452885 [Homo sapiens] (SEQ ID NO: 115)
CATCAACATATTCATCACCTCCCAAC[A/G]TTTTCTTTTGCCTCATTACAATTTT
rs17599556 [Homo sapiens] (SEQ ID NO: 116)
CGTACTATGTTAACAAGTGTGCCGAG[A/C]CAAATGTGTTTTCTCACCAGTTGTA
rs185425 [Homo sapiens] (SEQ ID NO: 117)
TGCCTGGATAGTCAGTTACAGACTTG[C/T]CAATTTCCAAGGGCTTTAAGTACTC
rs11035240 [Homo sapiens] (SEQ ID NO: 118)
TAAACTAAACACTTTCTTCCCAGAAG[C/T]CTCTGTCCTTGCTGTGTTTGATAAA
rs9693369 [Homo sapiens] (SEQ ID NO: 119)
AATATAATTGTCACATATACCTCCTC[C/T]GTCATAATATGCTTTCTTAGCTCTG
rs10781238 [Homo sapiens] (SEQ ID NO: 120)
ACACTCACCACAATAGAAGGGAGTAA[C/T]ATATAAAGCCCCAGCAATACGAAAT
rs9568011 [Homo sapiens] (SEQ ID NO: 121)
CCCAAATGCCTCAGAAAAATTTCAAC[C/T]GGGGGAATTTATCTTAGAGTTGTTC
rs11682846 [Homo sapiens] (SEQ ID NO: 122)
ATACTTCCTGAAGGAAGTTACATCTA[C/T]GCTAAATTCAAAGGTATAACAGATT
rs7650071 [Homo sapiens] (SEQ ID NO: 123)
AATGGGGCAATTGAGACAGGCCCTGA[A/G]GGATACTGAGGGTAGGGAGTGATGA
rs2574852 [Homo sapiens] (SEQ ID NO: 124)
GGAGGGCCTAGGCAAAGGCAACCGGC[A/G]TAGCACTGAGAGCACTTGAGGGCTG
rs11914753 [Homo sapiens] (SEQ ID NO: 125)
GACTTCTTATCTTTGGTTTTACAGGG[C/T]GGGTATGTTAGTGAGATACATCTGT
rs2469183 [Homo sapiens] (SEQ ID NO: 126)
GACCTTTGTTACTAAGAATTGAAGTG[G/T]AGAGACTAACAGAAGACAATAAGAA
rs274646 [Homo sapiens] (SEQ ID NO: 127)
CAGTCCCCACCCTGCCCTGCAGCCCC[C/T]GGGCAGGTTCCTCTCCTGCTTCGCT
rs13096022 [Homo sapiens] (SEQ ID NO: 128)
GATTGGATTGGCTTAGACAAATCAGG[A/G]TTAACTCAGTGAAAAGTCCAGAAAT
rs17738966 [Homo sapiens] (SEQ ID NO: 129)
GATACAAGATAAGGGAGGGAATGACT[A/G]TGAGGACTTTAGAGTATCCAAAGTA
rs6461176 [Homo sapiens] (SEQ ID NO: 130)
TTCCCAGACGTAAGAGTTTAGTGATC[A/C]ATTGTGTTAATAATAGCAATTGTCA
rs11138895 [Homo sapiens] (SEQ ID NO: 131)
AAGAATCCAGAAAAGAGAAGAAGATC[A/C]TTTGAGATAGAGTATTAGCAAGGCA
rs4809918 [Homo sapiens] (SEQ ID NO: 132)
GGAATGGGGGCACTGCCCAGATTTGT[A/G]GGTGGTGGATTGTGGGTGGAGGTCA
rs9479482 [Homo sapiens] (SEQ ID NO: 133)
TGCTGCATGCAGATTCTATCTCAAAA[C/T]AAAACACTCTGAAGATGTTCCAAGA
rs1294264 [Homo sapiens] (SEQ ID NO: 134)
CAAAATGTAGCAAAAAGTAAACACAG[C/T]GGTCATCCAGTTGCTGGTTTTCTCA
rs10788819 [Homo sapiens] (SEQ ID NO: 135)
GAGAAAGGCTTTAATTTGTGTAGAGC[C/T]TCCGTATGGTAGACTGGAGTTTTAT
rs4959923 [Homo sapiens] (SEQ ID NO: 136)
AGCAGGATGGAGGGAGAAGCGGAGGG[A/G]CTTGGTCCGCCACACGAAGTGGAAC
rs4905110 [Homo sapiens] (SEQ ID NO: 137)
ATAAATTTGTCATACGTGGTTATGGC[A/G]GGCCTAGGAAACGAATTCAGCTTGT
rs721087 [Homo sapiens] (SEQ ID NO: 138)
CCACAGATTTTCTCTCTCTCCATTGT[C/T]TTTACTGGGCTGTGTCCCCACTATG
rs10874468 [Homo sapiens] (SEQ ID NO: 139)
AAAAAAAAAAAAAAAAATCTGACCAC[A/G]TCAGCAAACAGGGAGAACCTTACAT
rs13384439 [Homo sapiens] (SEQ ID NO: 140)
ATAGAGAAGCATATTTAAAAATGTCC[A/G]AGGCGGGTGGATTGCCTGAGGGTGG
rs4416176 [Homo sapiens] (SEQ ID NO: 141)
TCTAGTTCTGAGAGTCTATTCTGTCA[C/T]GAAAGCTTGAAAGTTGCTGTGCATG
rs10519124 [Homo sapiens] (SEQ ID NO: 142)
ATGGCACATGCCACATGCCCGTTACA[A/G]CAACTTGAGGAACTCATACTGACTG
rs12962411 [Homo sapiens] (SEQ ID NO: 143)
AGGCAGTATTACCTAGATTCATTAGA[A/G]GGATTGGCAGCAGAAACAGCACTAA
rs6022029 [Homo sapiens] (SEQ ID NO: 144)
GAAACATACCATGGTGATACATTTAT[A/G]GGGCCTAATTATGTACTATTTTCAA
rs11627027 [Homo sapiens] (SEQ ID NO: 145)
CTTGCCGTCCTTATGAGGACACTCCT[C/T]ACAGTTTCTGCCACTGCACGGTCCT
rs6022039 [Homo sapiens] (SEQ ID NO: 146)
GGCCTTTGTAAATGTCATTCCTGGCC[C/T]TCTCACCTGGCGGATTCCTGCTGGC
rs10886048 [Homo sapiens] (SEQ ID NO: 147)
GTTTGGGCCTCTGCTCACCTTCTGAA[C/T]GGCTGGAACTTTCTATTAAAAATTC
rs4873815 [Homo sapiens] (SEQ ID NO: 148)
CTGAGGTGGTCTCTTAGATTCCTGGC[C/T]CCTAATGTACACACCCCTTCTTCCA
rs4832481 [Homo sapiens] (SEQ ID NO: 149)
GAGAAAGTCCTATAACAAATTGATGA[A/C]CTTAAGAGCAAAGTCTGAGGTCCCC
rs3809282 [Homo sapiens] (SEQ ID NO: 150)
AACATTTTGTTATGCTTAAATGTCTC[A/G]AAAATGAATTAGAGGCCCTAAAGGG
rs2297172 [Homo sapiens] (SEQ ID NO: 151)
TTGCTGTATATCAGTCTTTCGATTTC[C/T]TTTTGAGAATGGGAGCCTTAGTGCA
rs2255313 [Homo sapiens] (SEQ ID NO: 152)
CTTTTTTCCTCCTCAGCAAGTGACTA[C/T]CCTGAAAGCAATCATGTTTTCTTGT
rs2627468 [Homo sapiens] (SEQ ID NO: 153)
TATTTTCCCTTTGAAGCTCACCCCAG[C/T]ACGTATTGACAAGGACAATTGTAGG
rs12183587 has merged into rs9479478 [Homo sapiens] (SEQ ID
NO: 154)
CAAGGGACATTGCAAAAGCTAGCTTA[G/T]GGACTTCCCCATTCACAGGGAGAAG
rs10305860 [Homo sapiens] (SEQ ID NO: 155)
AGTTCATTACTCCCATTTCATTCATC[A/G]GCAAATACCGTATTGTGATGATAAT
rs30746 [Homo sapiens] (SEQ ID NO: 156)
GATATACGCAGCTGTTAAAATCATGC[A/G]TACAGGACTATTGGTTGAATAGTCC
rs11138885 [Homo sapiens] (SEQ ID NO: 157)
CATTGCAAGACTTCCAGGAGTGCATC[C/T]GTTTCTAATGTACAGTGCATAATTT
rs1294293 [Homo sapiens] (SEQ ID NO: 158)
TTTCTCCACTTTTCATTCTAGTTACA[A/C]CTAACTACTCATTGTTCCCTGAAAA
rs12115722 [Homo sapiens] (SEQ ID NO: 159)
GCTGGAAAGACATGCTTTTAAAAAAT[G/T]GTGCTAAATATGTATAACATACGAT
rs10997162 [Homo sapiens] (SEQ ID NO: 160)
CCTCCTAAGTCACATTCCTTGTCACT[G/T]ACTATCAAACATTCAAAATGTATCC
rs4778640 [Homo sapiens] (SEQ ID NO: 161)
GAATGAATGAATTCTAAGTCAATCCA[A/G]GAGTCTGATGATTTCTTGAAAAGGG
rs10110252 [Homo sapiens] (SEQ ID NO: 162)
TTATCACATTTTCTCAGACAATGTAA[C/T]AGGGGATGCTGCTTGTCCTCAACAT
rs12811136 [Homo sapiens] (SEQ ID NO: 163)
GATTCCTGCTTTTATTATTATGAATT[C/T]TCAGAGTAATTTCTCCCGCCTCCTG
rs17192980 [Homo sapiens] (SEQ ID NO: 164)
CTCTGTGGCTTCCTTAGATGTTAGAA[C/T]TGGGTTATGCAGAAGTCATTCAGTT
rs4811895 [Homo sapiens] (SEQ ID NO: 165)
ACTGAGAGTATGGAGTATGTCTCCGA[A/G]ATACATAGGTGATGTGTATTCTAGA
rs2519866 [Homo sapiens] (SEQ ID NO: 166)
AAATCCTGCCTCTACTCTATCACTTC[A/G]GGCAGGCAGGTCCTTAGGCTCTTTG
rs12266938 [Homo sapiens] (SEQ ID NO: 167)
GTCGCAAAACAAAACAAAACAAAACC[C/T]GCTCAAATCGTGTTAAAACAAGCAA
rs1896731[Homo sapiens] (SEQ ID NO: 168)
AAAATAAAAATTTGACCCAACATTAC[C/T]ACTGAGGAGGATGAACTTAAAATAC
rs10038113[Homo sapiens] (SEQ ID NO: 169)
GCAGCAATCTAGGTTTGGCCATGTAG[C/T]GGAAGACAAGGTCATGGGGCATCAA
rs7704909[Homo sapiens] (SEQ ID NO: 170)
TATATATTTATCTATCTATATGTAAA[A/C/G/T]ATAATCAATCAACCAGAAGGACATT
rs4327572[Homo sapiens] (SEQ ID NO: 171)
tattttataaatacttataaaGCAAA[A/C/G/T]AAAACAGCAAAATATGAAAAAGACA
rs12518194[Homo sapiens] (SEQ ID NO: 172)
TGGCATATAAACAGAGGATCTGGGGC[A/G]TACAACTTGATTTCAACTTTTTACA
rs4307059[Homo sapiens] (SEQ ID NO: 173)
TAGCTTTCACTGATGTGTCCGAATTG[C/T]TTCATGTAACCAGGATATTTTCCAT

### REFERENCES

1. American Psychological Association (1994) Diagnostic and Statistical Manual of Mental Disorders, (American Psychological Association, Washington, D.C.),
2. Volkmar F R (1991) DSM-IV in progress. autism and the pervasive developmental disorders. Hosp Community Psychiatry 42: 33-5.
3. Bailey A, et al (1995) Autism as a strongly genetic disorder: Evidence from a british twin study. Psychol Med 25: 63-77.
4. Feng Y, et al (1995) Translational suppression by trinucleotide repeat expansion at FMR1. Science 268: 731-4.
5. Amir R E, et al (1999) Rett syndrome is caused by mutations in X-linked MECP2, encoding methyl-CpG-binding protein 2. Nat Genet 23: 185-8.
6. Kim S J & H. CE, Jr (2000) Novel de novo nonsense mutation of MECP2 in a patient with rett syndrome. Hum Mutat 15: 382-3.
7. Smalley S L, Burger F & Smith M (1994) Phenotypic variation of tuberous sclerosis in a single extended kindred. J Med Genet 31: 761-765.
8. Zhou C Y, et al (1995) Physical analysis of the tuberous sclerosis region in 9q34. Genomics 25: 304-308.
9. Wang K, et al (2009) Common genetic variants on 5p14.1 associate with autism spectrum disorders. Nature 459: 528-533.
10. Ma D, et al (2009) A genome-wide association study of autism reveals a common novel risk locus at 5p14.1. Ann Hum Genet 73: 263-273.
11. Weiss L A, et al (2009) A genome-wide linkage and association scan reveals novel loci for autism. Nature 461: 802-808.
12. Anney R, et al (2010) A genome-wide scan for common alleles affecting risk for autism. Hum Mol Genet 19: 4072-4082.
13. Hu V W & Steinberg M E (2009) Novel clustering of items from the autism diagnostic interview-revised to define phenotypes within autism spectrum disorders. Autism Res 2: 67-77.
14. Hu V W, et al (2009) Gene expression profiling differentiates autism case-controls and phenotypic variants of autism spectrum disorders: Evidence for circadian rhythm dysfunction in severe autism. Autism Res 2: 78-97.
15. Pinto D, et al (2010) Functional impact of global rare copy number variation in autism spectrum disorders. Nature 466: 368-372.
16. Nurmi E L, et al (2003) Exploratory subsetting of autism families based on savant skills improves evidence of genetic linkage to 15q11-q13. J Am Acad Child Adolesc Psychiatry 42: 856-63.
17. Nijmeijer J S, et al (2010) Identifying loci for the overlap between attention-deficit/hyperactivity disorder and autism spectrum disorder using a genome-wide QTL linkage approach. J Am Acad Child Adolesc Psychiatry 49: 675-685.
18. Ronald A, et al (2010) A genome-wide association study of social and non-social autistic-like traits in the general population using pooled DNA, 500 K SNP microarrays and both community and diagnosed autism replication samples. Behav Genet 40: 31-45.
19. Cannon D, et al (2010) Genome-wide linkage analyses of two repetitive behavior phenotypes in utah pedigrees with autism spectrum disorders. Molecular Autism 1:
20. Duvall J A, et al (2007) A quantitative trait locus analysis of social responsiveness in multiplex autism families. Am J Psychiatry 164: 656-62.
21. Coon H, et al (2010) Genome-wide linkage using the social responsiveness scale in utah autism pedigrees. Molecular Autism 1:
22. St. Pourcain B, et al (2010) Association between a high-risk autism locus on 5p14 and social communication spectrum phenotypes in the general population. Am J Psychiatry 167: 1364-1372.
23. Suzuki T, et al (2003) Association of a haplotype in the serotonin 5-HT4 receptor gene (HTR4) with japanese schizophrenia. American Journal of Medical Genetics—Neuropsychiatric Genetics 121 B: 7-13.
24. Kato T, Kuratomi G & Kato N (2005) Genetics of bipolar disorder. Drugs of Today 41: 335-344.
25. Li J, et al (2007) Association between polymorphisms in serotonin transporter gene and attention deficit hyperactivity disorder in chinese han subjects. American Journal of Medical Genetics, Part B: Neuropsychiatric Genetics 144: 14-19.
26. Vincent J B, et al (2009) Characterization of a de novo translocation t(5; 18) (q33.1; q12.1) in an autistic boy identifies a breakpoint close to SH3TC2, ADRB2, and HTR4 on 5q, and within the desmocollin gene cluster on 18q. American Journal of Medical Genetics, Part B: Neuropsychiatric Genetics 150: 817-826.
27. Serova L I, et al (1999) Heightened transcription for enzymes involved in norepinephrine biosynthesis in the rat locus coeruleus by immobilization stress. Biol Psychiatry 45: 853-862.
28. Tegeder I, et al (2008) Reduced hyperalgesia in homozygous carriers of a GTP cyclohydrolase 1 haplotype. European Journal of Pain 12: 1069-1077.
29. Tegeder I, et al (2006) GTP cyclohydrolase and tetrahydrobiopterin regulate pain sensitivity and persistence. Nat Med 12: 1269-1277.
30. Campbell C M, et al (2009) Polymorphisms in the GTP cyclohydrolase gene (GCH1) are associated with ratings of capsaicin pain. Pain 141: 114-118.
31. Cao L, et al (2010) Four novel mutations in the GCH1 gene of chinese patients with dopa-responsive dystonia. Movement Disorders 25: 755-760.
32. Purcell S, et al (2007) PLINK: A tool set for whole-genome association and population-based linkage analyses. Am J Hum Genet 81: 559-575.

1. A screening method for detecting in a subject a propensity or increased risk for developing an autistim spectrum disorder (ASD) comprising detecting the presence of at least one single nucleotide polymorphism (SNP) in at least one target polynucleotide in a subject wherein the SNP comprises rs2277049, rs757099, rs7785107, rs7725785, rs2287581, rs1231339, rs2180055, rs11671930, rs7950390, rs12266938, rs3861787, rs1827924, rs17738966, rs317985, rs730168, rs10519124, rs6482516, or rs2297172, or any combination thereof, and wherein detecting the presence of the SNP in the subject is indicative of a propensity or increased risk of developing an ASD.

2. The method of claim 1 wherein detecting the presence of rs2277049, rs757099, rs7785107, rs7725785, rs2287581, rs1231339, rs2180055, rs11671930, rs7950390, rs12266938, rs3861787, rs1827924, rs17738966, rs317985, rs730168, rs10519124, rs6482516, and rs2297172 in the subject is indicative of a propensity or increased risk of developing an ASD.

3. The method of claim 1 wherein detecting the presence of rs2277049 rs757099, rs7785107, rs7725785, rs2287581, rs1231339, rs2180055, and rs11671930.

4. The method of claim 1 wherein the SNPs comprise rs7785107, rs7950390, rs12266938, and rs3861787 in the subject is indicative of a propensity or increased risk of developing an ASD.

5. The method of claim 1 wherein detecting the presence of rs1827924, rs17738966, rs7950390, rs3861787, rs317985, and rs7725785 in the subject is indicative of a propensity or increased risk of developing an ASD.

6. The method of claim 1 wherein detecting the presence of rs12266938, rs730168, rs10519124, rs6482516, rs11671930, rs2297172, rs317985, rs1827924, rs1231339, rs757099, and rs7725785 in the subject is indicative of a propensity or increased risk of developing an ASD.

7. The method of claim 1 wherein detecting the presence of rs317985, rs7785107, rs11671930, rs7950390, rs12266938, rs3861787, rs7725785, rs1827924, rs1231339, and rs757099 in the subject is indicative of a propensity or increased risk of developing an ASD.

8. The method of claim 1 wherein the ASD comprises autistic disorder, pervasive developmental disorder-not otherwise specified (PDD-NOS), including atypical autism, Asperger's Disorder, or a combination thereof.

9. The method of claim 1 wherein the SNPs are detected by
(a) preparing samples of control and experimental DNA, wherein the experimental DNA is generated from a nucleic acid sample isolated from the subject and the control DNA is generated from a nucleic acid sample isolated from a nonautistic individual;
(b) applying the prepared samples to one or more microarrays comprising a plurality of different oligonucleotides having specificity for at least one allele of the SNPs under conditions suitable for hybridization between (i) the oligonucleotides and the control DNA and (ii) the oligonucleotides and the experimental DNA; and
(c) identifying the oligonucleotides on the microarray which display differential hybridization to the experimental DNA relative to the control DNA,
wherein a significant differential hybridization between the control and experimental DNA to the oligonucleotides having specificity for the SNPs is indicative of a subject with a propensity or increased risk of having or developing the ASD.

10. The method of claim 3 wherein the presence of the SNPs is indicative of a subject having a propensity or increased risk of developing an ASD subtype with higher serverity scores on spoken language items on the ADI-R (Language Impared subtype).

11. The method of claim 4 wherein the presence of the SNPs is indicative of a subject having a propensity or increased risk of developing an ASD subtype with intermediate severity scores on the ADI-R (Intermediate subtype).

12. The method of claim 5 wherein the presence of the SNPs is indicative of a subject having a propensity or increased risk of developing an ASD subtype with moderate severity scores n the ADI-R (Moderate subtype).

13. The method of claim 6 wherein the presence of the SNPs is indicative of a subject having a propensity or increased risk of developing an ASD subtype with lower severity scores on the ADI-R (Mild subtype).

14. (canceled)

15. A biomarker for the diagnosis of autism spectrum disorders comprising at least one language impairment quantitative trait loci-specific single nucleotide polymorphism, at least one non-verbal communication quantitative trait loci-specific single nucleotide polymorphism, at least one play skills quantitative trait loci-specific single nucleotide polymorphism, at least one insistence on sameness/rituals quantitative trait loci-specific single nucleotide polymorphism, and/or at least one social skills and development quantitative trait loci-specific single nucleotide polymorphism comprising a biomarker set forth in Table 1 or Table 7, variants, mutants, alleles or complementary sequences thereof, or any combination thereof.

16. (canceled)

17. A biomarker for the diagnosis of autism spectrum disorders comprising
(a) at least one combined quantitative trait loci-specific and ASD sub-type specific single nucleotide polymorphism set forth as rs2277049, rs757099, rs7785107, rs7725785, rs2287581, rs1231339, rs2180055, rs11671930, rs7950390, rs12266938, rs3861787, rs1827924, rs17738966, rs317985, rs730168, rs10519124, rs6482516, or rs2297172, or variants, mutants, alleles or complementary sequences thereof, or any combination thereof, or
(b) at least one combined quantitative trait loci-specific and ASD sub-type language impaired-specific single nucleotide polymorphism set forth as rs12407665, rs17828521, rs9474831, rs6454792, rs10183984, rs11969265, rs1231339, rs10806416, rs7785107, rs2277049, rs757099, rs7725785, rs758158, rs2287581, rs17830215, rs2180055, rs12893752, variants, mutants, alleles or complementary sequences thereof, or any combination thereof.

18. (canceled)

19. The biomarker according to claim 17, wherein the autism spectrum disorder comprises autistic disorder, pervasive developmental disorder-not otherwise specified (PDD-NOS), including atypical autism, Asperger's Disorder, or a combination thereof.

20. (canceled)

21. (canceled)

22. A method for identifying biomarkers for the diagnosis of autism spectrum disorders comprising (a) performing quantitative trait association analysis for at least one category of symptoms or related quantitative traits, to identify filtered set of single nucleotide polymorphisms that are associated with each quantitative trait; (b) performing case-control association analysis with each set of trait-associated single nucleotide polymorphisms in which cases are both combined and divided into from at least one to at least four ASD subtypes to identify trait associated single nucleotide polymorphisms that are subtype-dependent with a Bonferroni significance of P<0.05; (c) performing case control association analysis with the combined set of Bonferroni significant single nucleotide polymorphisms from analysis in step (b) to identify those novel ASD subtype-associated single nucleotide polymorphisms that are associated with each quantitative trait and those novel ASD subtype-associated quantitative trait loci that are replicated in a second subtype.

23. The method according to claim 22, wherein the quantitative severity criteria are assessed across at least one category of behavioral symptoms or quantitative traits of ASD subtypes comprising language deficits, deficits in nonverbal communication, under developed playful skills, delayed social development, and insistence on sameness/rituals, separately or in combination with measuring the level of differential gene expression in one or more of the biomarker-associated genes listed in Table 1 or Table 7, or any combination thereof.

24. The method according to claim 22, wherein the case-control association analysis of step (b) comprises a cluster analysis to divide the autistic cases into four phenotypic subgroups according to symptomatic severity profiles derived from the one to one hundred and twenty three items listed on the ADI-R assessments in Table 9 to reduce the behavioral/symptomatic and heterogeneity genetic heterogeneity among the cases within each subgroup.

25.-36. (canceled)

37. The biomarker according to claim 15, wherein the autism spectrum disorder comprises autistic disorder, pervasive developmental disorder-not otherwise specified (PDD-NOS), including atypical autism, Asperger's Disorder, or a combination thereof.


SEQUENCE LISTING (167 sequences)

SEQ ID NO: 1 - DNA, 52 bp, Homo sapiens
tagaaccagcacacattggccaaatabggcccatggctctcgaatggtcttt

SEQ ID NO: 2 - DNA, 52 bp, Homo sapiens
acctacaatcaaattgttgtcttctcyttactgatctttgaaacacctttaa

SEQ ID NO: 3 - DNA, 52 bp, Homo sapiens
aactctcacacacattgacgctgtttycttctcagctattcaaagtccattt

SEQ ID NO: 4 - DNA, 52 bp, Homo sapiens
tatcacagatgtactgtgctgatagaraagtctgagctattggatttgccag

SEQ ID NO: 5 - DNA, 52 bp, Homo sapiens
gctgttgttgaataaatatactatacrtgttaatcagatccattagattgat

SEQ ID NO: 6 - DNA, 52 bp, Homo sapiens
agtataatgtgagatgatgctgcatayagccatggtcagaaacggggaaaag

SEQ ID NO: 7 - DNA, 52 bp, Homo sapiens
tctaacaaaatgtcttaggctgggtamtttataaggctaagacatttacttc

SEQ ID NO: 8 - DNA, 52 bp, Homo sapiens
agatggctgtgctggcacagagcatgyccctccagttctccaagatggagca

SEQ ID NO: 9 - DNA, 52 bp, Homo sapiens
tcctatagctttagctctaaatcagtkaattccaattttttatattatactt

SEQ ID NO: 10 - DNA, 52 bp, Homo sapiens
tctcctctttttaccctgtattctttmttttatttccttatttttattctct

SEQ ID NO: 11 - DNA, 52 bp, Homo sapiens
tctcttgtgaattgtagtaatggaagyactactttaaaacttcttcagcaga

SEQ ID NO: 12 - DNA, 52 bp, Homo sapiens
ctaatattatttattcatttaggaacmccatgcaaagttgatcagacagtaa

SEQ ID NO: 13 - DNA, 52 bp, Homo sapiens
tccctggggagggagatctgagctccrggttagaagtttggtagaggttagc

SEQ ID NO: 14 - DNA, 52 bp, Homo sapiens
aagtccaagagcttgaggttgggggtrggggaagaagtgacatatttaaagc

SEQ ID NO: 15 - DNA, 52 bp, Homo sapiens
gtgttttttaaagtgatttcctggcaycctacaaacagtagcattttaatcc

SEQ ID NO: 16 - DNA, 52 bp, Homo sapiens
acatggttggagtccttcagagggtcyagctcaccagaggacgcccagagtc

SEQ ID NO: 17 - DNA, 52 bp, Homo sapiens
gttatccccagggagggttgctagcaygttgtcacctttcaatatggcagat

SEQ ID NO: 18 - DNA, 52 bp, Homo sapiens
tacatcagctcagctggaacagacccrtccaaagaggagaatttttgttttg

SEQ ID NO: 19 - DNA, 52 bp, Homo sapiens
ggggacccaccagggaacaggtggctygagggcggggggagcatgaaggtaa

SEQ ID NO: 20 - DNA, 52 bp, Homo sapiens
aaagatattgaagctcacaacataaahaagaacaagctcgctaatatgtcta

SEQ ID NO: 21 - DNA, 52 bp, Homo sapiens
catgggattgtggagacaggaatcccrgaatatcaatacaataattcccagg

SEQ ID NO: 22 - DNA, 52 bp, Homo sapiens
atcagagcaagagagaaccatgactgytggaatatcacagaaaaagtgatgg

SEQ ID NO: 23 - DNA, 52 bp, Homo sapiens
aaaagtaccgcacatcaaaacagtagytgttttcttcaacccacacgcgcac

SEQ ID NO: 24 - DNA, 52 bp, Homo sapiens
cttataaagagccagatcataaatacrtagggtttgtgggccatatggtctc

SEQ ID NO: 25 - DNA, 52 bp, Homo sapiens
tttgaaatacctggagtggttccactrtgacaaccagatcttgactcttaca

SEQ ID NO: 26 - DNA, 52 bp, Homo sapiens
tattaaataaggtggaaaagacgtaaygtgtggccttgtttcaacgtgccaa

SEQ ID NO: 27 - DNA, 52 bp, Homo sapiens
tctcaacaactttcttgaatgcactcytcacttccttgttcctcagactata

SEQ ID NO: 28 - DNA, 52 bp, Homo sapiens
tgtggaagaaggccctgaaggaaaggrcctgggttccaggccaggctctgtc

SEQ ID NO: 29 - DNA, 52 bp, Homo sapiens
cttcaaacaagacttccaagaccaaaycaaattctcagggatcattttcttc

SEQ ID NO: 30 - DNA, 52 bp, Homo sapiens
tctcaaggaagaatataaataagtcaytgactatcatttgcactctgatctc

SEQ ID NO: 31 - DNA, 52 bp, Homo sapiens
ttggaaccatagcaggatctgatagtraccctgaagctggaaggaaccttgg

SEQ ID NO: 32 - DNA, 52 bp, Homo sapiens
gagcttgggctctgggaagaggtgcaygtcatttctacatgtacaacctaag

SEQ ID NO: 33 - DNA, 52 bp, Homo sapiens
gcatcatccagcctagggttttactaycatcttagggagagcagcacggcat

SEQ ID NO: 34 - DNA, 52 bp, Homo sapiens
cagacagagaagcagcagcaaatcagyggaggccaggattgatagcttcccc

SEQ ID NO: 35 - DNA, 52 bp, Homo sapiens
cttgagattgtgttggtgttaatatayatagcctcactttgagggcaggtga

SEQ ID NO: 36 - DNA, 52 bp, Homo sapiens
  modified_base (27)..(27): a, c, t or g
gaagaatcaataagtcgcttttggctntaaaatggctcctgagcagtcacct

SEQ ID NO: 37 - DNA, 52 bp, Homo sapiens
ctaggactaaaggcaatatagactacygtgatactatctagttcgcgaaagt

SEQ ID NO: 38 - DNA, 52 bp, Homo sapiens
acacttttgcatccacatggtgtctcracacagctaagtcctcagtcataac

SEQ ID NO: 39 - DNA, 52 bp, Homo sapiens
acatagaggtgacacacagggctgaargtgtgggtgggttttcaagttggca

SEQ ID NO: 40 - DNA, 52 bp, Homo sapiens
agacatacctttgcctacaacacaaaytcattaggtttccttccttagattt

SEQ ID NO: 41 - DNA, 52 bp, Homo sapiens
gcaaatatgtgcctgttacaaacttgycttcatctgtgtgtacagccattca

SEQ ID NO: 42 - DNA, 52 bp, Homo sapiens
aagcctgagttccagcctttgctgttycaggagtgactgttccattctgagt

SEQ ID NO: 43 - DNA, 52 bp, Homo sapiens
aggtaaaaaacacaacagatgccaatrgcaagagtgtctagatattgaaatg

SEQ ID NO: 44 - DNA, 52 bp, Homo sapiens
aagtcccataaattccatttcttgaargaaaaggccatgagtcagtatttga

SEQ ID NO: 45 - DNA, 52 bp, Homo sapiens
ttagtttccgttatttacgtgtgtgaytcatggaaatgtttatgtcttgccc

SEQ ID NO: 46 - DNA, 52 bp, Homo sapiens
ttaagaatgggttttcaaacaaacttkctagtctgtttttgaaaagtcaagg

SEQ ID NO: 47 - DNA, 52 bp, Homo sapiens
aagacacatgtcacacacatggcacgmtcaacaatgtcagtctagtcatagg

SEQ ID NO: 48 - DNA, 52 bp, Homo sapiens
gatgacactgatggtgacgatactgaygatagtaataacacttactgaatac

SEQ ID NO: 49 - DNA, 51 bp, Homo sapiens
tccagctggcacaagggtctgtggaamgtgcatacgtgttcccagctctaa

SEQ ID NO: 50 - DNA, 52 bp, Homo sapiens
tcctttctaagaagatccttcagcctrtattctgggcatactttctcacaac

SEQ ID NO: 51 - DNA, 52 bp, Homo sapiens
gatgaatctgatcaatgatgatgaatrgactatgttacacataacgtcatag

SEQ ID NO: 52 - DNA, 52 bp, Homo sapiens
aacagctccccatacccaactatctaycctaaaatgacatgccactagtgaa

SEQ ID NO: 53 - DNA, 52 bp, Homo sapiens
tgtgtccaagaccttctggcccttgcrtgtaagatgtgcttcttccctctag

SEQ ID NO: 54 - DNA, 52 bp, Homo sapiens
  modified_base (27)..(27): a, c, t or g
agctctcttggctccttcctatgtatnatgataactgatccaattgatcctt

SEQ ID NO: 55 - DNA, 52 bp, Homo sapiens
tgaagacctaagtattgactagttgtktatgttggaccacattttaatttca

SEQ ID NO: 56 - DNA, 52 bp, Homo sapiens
cactgttggtctccatgttgtcaaagyatagagcaatgagagtttttgacca

SEQ ID NO: 57 - DNA, 52 bp, Homo sapiens
tccaagggagaggtcacaggtcctcamcttttgagcagaggagtgtctgaca

SEQ ID NO: 58 - DNA, 52 bp, Homo sapiens
agggctcaccagtttgagaactgcagyagccttcgacagccttcctgaatca

SEQ ID NO: 59 - DNA, 52 bp, Homo sapiens
ctgctggcatgctccattctatccacrtgcccggtcacatggagactttcag

SEQ ID NO: 60 - DNA, 52 bp, Homo sapiens
gacctctcagaagccttgccagaaacrtcaatggatcccatcacgggagtcg

SEQ ID NO: 61 - DNA, 52 bp, Homo sapiens
cttatcttgccatagctttaggatatytgaagaatgtgttcatagaaaatga

SEQ ID NO: 62 - DNA, 52 bp, Homo sapiens
aaaacactgtttaacatctgaagttcrtttgcaagaagagtagatgagctag

SEQ ID NO: 63 - DNA, 52 bp, Homo sapiens
aagtctgcgggagaaatggcattaackggcaataaatgggactgacagaaat

SEQ ID NO: 64 - DNA, 52 bp, Homo sapiens
cggacatctgccggttggtggtaaagytgttgattttaggaaattctagaga

SEQ ID NO: 65 - DNA, 52 bp, Homo sapiens
ctttcatttcctccttgtgccaaggayttcaaggccagacataagagtggga

SEQ ID NO: 66 - DNA, 52 bp, Homo sapiens
cccaaataaatctttaaagccaaaaamcagatttacatgtgtgtcctgtgtt

SEQ ID NO: 67 - DNA, 52 bp, Homo sapiens
ctgtgcctgtttatttcacggatttcrggttaaccattacagaaaggccatg

SEQ ID NO: 68 - DNA, 52 bp, Homo sapiens
aatttcccattgacccaaaatcattgyggggcaattcaaatttaacaggtgg

SEQ ID NO: 69 - DNA, 52 bp, Homo sapiens
  modified_base (27)..(27): a, c, t or g
actgctaacaaatagcagtgttttgantttcctgttctttctacctcttcaa

SEQ ID NO: 70 - DNA, 52 bp, Homo sapiens
ccctgcattacactagtcattatatcyatttcaagcaaaagggattttaaaa

SEQ ID NO: 71 - DNA, 52 bp, Homo sapiens
tggtagttgtttggcataaacacaacrgtctaaatggatggtgacaggcaac

SEQ ID NO: 72 - DNA, 52 bp, Homo sapiens
  modified_base (27)..(27): a, c, t or g
tttctgtctggttaagtgaagacgaangaatgaagaatcacagtgttcttac

SEQ ID NO: 73 - DNA, 52 bp, Homo sapiens
  modified_base (27)..(27): a, c, t or g
atctgaccactcttcaaaaggaggtanatgtgacagcagctggaaatttcca

SEQ ID NO: 74 - DNA, 52 bp, Homo sapiens
tcagaagcttgggagtctccgctccckcagactcccataccagaagcaggaa

SEQ ID NO: 75 - DNA, 52 bp, Homo sapiens
ggaccttgctagaggacttaaatgagkgtggagccacaagatggacagagcc

SEQ ID NO: 76 - DNA, 52 bp, Homo sapiens
ttttgactggctattggtcagttcctmgtctttcccaatctgaaaaatgggt

SEQ ID NO: 77 - DNA, 52 bp, Homo sapiens
aatgctagtagcagagattttactttragccagagtcaaatccaatgtaggg

SEQ ID NO: 78 - DNA, 52 bp, Homo sapiens
ttgatgcaataatatctgttatgtaakagaagctcaacaaatttttatttat

SEQ ID NO: 79 - DNA, 52 bp, Homo sapiens
attcctactattagcagaaattgcagyattcatacccatagccaaccctggg

SEQ ID NO: 80 - DNA, 52 bp, Homo sapiens
agaatgtactcaaaggcagctttagaractaaatcattatagaacatccatt

SEQ ID NO: 81 - DNA, 52 bp, Homo sapiens
gagtctgatcactcgtttactgacaartaaaacatacctgtcccatgtctcc

SEQ ID NO: 82 - DNA, 52 bp, Homo sapiens
tactttaaatagtgtttaagagcatargatttagagcctgaaacacctgtgt

SEQ ID NO: 83 - DNA, 52 bp, Homo sapiens
taataggggtgatatgtgctgaaaagmtgaaagtagtgtaagcattttttga

SEQ ID NO: 84 - DNA, 52 bp, Homo sapiens
ttttagaactgtcaactcacttccacrtgtattgggtgtctataccattatt

SEQ ID NO: 85 - DNA, 52 bp, Homo sapiens
gtatagaatataacagcttcatcagakaatatattcttaaaataatctattt

SEQ ID NO: 86 - DNA, 52 bp, Homo sapiens
atatattaccagccttttctaacccaygaaaggactcacactggagagaaac

SEQ ID NO: 87 - DNA, 52 bp, Homo sapiens
attgttgggcatgactcaattgagagrgaatagacccatgagaatcagatac

SEQ ID NO: 88 - DNA, 52 bp, Homo sapiens
gcactccagggaagtctttggaattayagctataggaaaacaagtaaaatgc

SEQ ID NO: 89 - DNA, 52 bp, Homo sapiens
ccttgatcattatcctgagtgactttrtccttaaagtgactatctttttaac

SEQ ID NO: 90 - DNA, 52 bp, Homo sapiens
tggctctatcaaatactgttatattcrtattacttgtgaataacctgaggtc

SEQ ID NO: 91 - DNA, 52 bp, Homo sapiens
gaggttccaaaattctcaattcacagygaccacagtgtctcagtaatttttt

SEQ ID NO: 92 - DNA, 52 bp, Homo sapiens
tgcaggtatgaaaaaaatgcttcaaaragcaattactaacacacttgaaaca

SEQ ID NO: 93 - DNA, 52 bp, Homo sapiens
cctaagccagagcctgtcacatgatargttcaaaataaatattggtttaatg

SEQ ID NO: 94 - DNA, 52 bp, Homo sapiens
tcttcactctctagcacatacctgatragacttagcagaagtaaactccttc

SEQ ID NO: 95 - DNA, 52 bp, Homo sapiens
accacccattctaaggatcacactatyagcatatactcgttttaattaaaga

SEQ ID NO: 96 - DNA, 52 bp, Homo sapiens
tattgaatattcagcagcttctcacartctagagaactattgaaggctctcc

SEQ ID NO: 97 - DNA, 52 bp, Homo sapiens
tttgcattgctgcttctcttcaccccrtggaggctatgtcaccctaactatc

SEQ ID NO: 98 - DNA, 52 bp, Homo sapiens
agtatttactgtgattcgttgaatccrtgaagaactaatccaactctccagg

SEQ ID NO: 99 - DNA, 52 bp, Homo sapiens
tcactgaaaagtttgtgggagcctggyggttccagggtgcacaccactcctt

SEQ ID NO: 100 - DNA, 52 bp, Homo sapiens
atcacctccctcacagcccagctccaygttcacccccaccaggaagtcttcc

SEQ ID NO: 101 - DNA, 52 bp, Homo sapiens
aaattatatgtgccagaaatttaacargagtgcagggtttatgctggaaaag

SEQ ID NO: 102 - DNA, 52 bp, Homo sapiens
taagcaggtaaaaggtttagcagtgtrtttgtcaggtattcagtaaatattg

SEQ ID NO: 103 - DNA, 52 bp, Homo sapiens
attaatgaaattcccaagagaaagtcrttgcagaaagttttaatcttacaga

SEQ ID NO: 104 - DNA, 52 bp, Homo sapiens
tactatgttatgtatattttaccacartactaagaagaataactaatgcccc

SEQ ID NO: 105 - DNA, 52 bp, Homo sapiens
cagccaggaatctaggatggttagaaraaaagatatttctctctaacatacc

SEQ ID NO: 106 - DNA, 52 bp, Homo sapiens
taggcttgatgagaatataatcttagkcttgaaggctttaaaggggaagaaa

SEQ ID NO: 107 - DNA, 52 bp, Homo sapiens
attgtaaaggtaaaatatatctttacrttggagagatctggtggtctctacc

SEQ ID NO: 108 - DNA, 52 bp, Homo sapiens
tggaagagtttatgtgagtttgtactrttactttcttcagtgattgatagaa

SEQ ID NO: 109 - DNA, 52 bp, Homo sapiens
tgtctttattagcagtatgagagcagrcaaatacacctacctaaaattaagc

SEQ ID NO: 110 - DNA, 52 bp, Homo sapiens
cagggcagatgaagtcataaaagctgrgctccctgtgttctgaagatgacca

SEQ ID NO: 111 - DNA, 52 bp, Homo sapiens
gcacattgcatatacggagagcatagkagacataatgaccatggatagaaaa

SEQ ID NO: 112 - DNA, 52 bp, Homo sapiens
tccaaacatcaatatttaagttaattrtatgctgttactactcaggacttca

SEQ ID NO: 113 - DNA, 52 bp, Homo sapiens
ctgcactcacatccccggggatcacaratgcaaatacagcttatgcatggga

SEQ ID NO: 114 - DNA, 52 bp, Homo sapiens
cacccaaaggtgaaggtgcactgggaraaaatgaagttgcgtgggggcatca

SEQ ID NO: 115 - DNA, 52 bp, Homo sapiens
catcaacatattcatcacctcccaacrttttcttttgcctcattacaatttt

SEQ ID NO: 116 - DNA, 52 bp, Homo sapiens
cgtactatgttaacaagtgtgccgagmcaaatgtgttttctcaccagttgta

SEQ ID NO: 117 - DNA, 52 bp, Homo sapiens
tgcctggatagtcagttacagacttgycaatttccaagggctttaagtactc

SEQ ID NO: 118 - DNA, 52 bp, Homo sapiens
taaactaaacactttcttcccagaagyctctgtccttgctgtgtttgataaa

SEQ ID NO: 119 - DNA, 52 bp, Homo sapiens
aatataattgtcacatatacctcctcygtcataatatgctttcttagctctg

SEQ ID NO: 120 - DNA, 52 bp, Homo sapiens
acactcaccacaatagaagggagtaayatataaagccccagcaatacgaaat

SEQ ID NO: 121 - DNA, 52 bp, Homo sapiens
cccaaatgcctcagaaaaatttcaacygggggaatttatcttagagttgttc

SEQ ID NO: 122 - DNA, 52 bp, Homo sapiens
atacttcctgaaggaagttacatctaygctaaattcaaaggtataacagatt

SEQ ID NO: 123 - DNA, 52 bp, Homo sapiens
aatggggcaattgagacaggccctgarggatactgagggtagggagtgatga

SEQ ID NO: 124 - DNA, 52 bp, Homo sapiens
ggagggcctaggcaaaggcaaccggcrtagcactgagagcacttgagggctg

SEQ ID NO: 125 - DNA, 52 bp, Homo sapiens
gacttcttatctttggttttacagggygggtatgttagtgagatacatctgt

SEQ ID NO: 126 - DNA, 52 bp, Homo sapiens
gacctttgttactaagaattgaagtgkagagactaacagaagacaataagaa

SEQ ID NO: 127 - DNA, 52 bp, Homo sapiens
cagtccccaccctgccctgcagccccygggcaggttcctctcctgcttcgct

SEQ ID NO: 128 - DNA, 52 bp, Homo sapiens
gattggattggcttagacaaatcaggrttaactcagtgaaaagtccagaaat

SEQ ID NO: 129 - DNA, 52 bp, Homo sapiens
gatacaagataagggagggaatgactrtgaggactttagagtatccaaagta

SEQ ID NO: 130 - DNA, 52 bp, Homo sapiens
ttcccagacgtaagagtttagtgatcmattgtgttaataatagcaattgtca

SEQ ID NO: 131 - DNA, 52 bp, Homo sapiens
aagaatccagaaaagagaagaagatcmtttgagatagagtattagcaaggca

SEQ ID NO: 132 - DNA, 52 bp, Homo sapiens
ggaatgggggcactgcccagatttgtrggtggtggattgtgggtggaggtca

SEQ ID NO: 133 - DNA, 52 bp, Homo sapiens
tgctgcatgcagattctatctcaaaayaaaacactctgaagatgttccaaga

SEQ ID NO: 134 - DNA, 52 bp, Homo sapiens
caaaatgtagcaaaaagtaaacacagyggtcatccagttgctggttttctca

SEQ ID NO: 135 - DNA, 52 bp, Homo sapiens
gagaaaggctttaatttgtgtagagcytccgtatggtagactggagttttat

SEQ ID NO: 136 - DNA, 52 bp, Homo sapiens
agcaggatggagggagaagcggagggrcttggtccgccacacgaagtggaac

SEQ ID NO: 137 - DNA, 52 bp, Homo sapiens
ataaatttgtcatacgtggttatggcrggcctaggaaacgaattcagcttgt

SEQ ID NO: 138 - DNA, 52 bp, Homo sapiens
ccacagattttctctctctccattgtytttactgggctgtgtccccactatg

SEQ ID NO: 139 - DNA, 52 bp, Homo sapiens
aaaaaaaaaaaaaaaaatctgaccacrtcagcaaacagggagaaccttacat

SEQ ID NO: 140 - DNA, 52 bp, Homo sapiens
atagagaagcatatttaaaaatgtccraggcgggtggattgcctgagggtgg

SEQ ID NO: 141 - DNA, 52 bp, Homo sapiens
tctagttctgagagtctattctgtcaygaaagcttgaaagttgctgtgcatg

SEQ ID NO: 142 - DNA, 52 bp, Homo sapiens
atggcacatgccacatgcccgttacarcaacttgaggaactcatactgactg

SEQ ID NO: 143 - DNA, 52 bp, Homo sapiens
aggcagtattacctagattcattagarggattggcagcagaaacagcactaa

SEQ ID NO: 144 - DNA, 52 bp, Homo sapiens
gaaacataccatggtgatacatttatrgggcctaattatgtactattttcaa

SEQ ID NO: 145 - DNA, 52 bp, Homo sapiens
cttgccgtccttatgaggacactcctyacagtttctgccactgcacggtcct

SEQ ID NO: 146 - DNA, 52 bp, Homo sapiens
ggcctttgtaaatgtcattcctggccytctcacctggcggattcctgctggc

SEQ ID NO: 147 - DNA, 52 bp, Homo sapiens
gtttgggcctctgctcaccttctgaayggctggaactttctattaaaaattc

SEQ ID NO: 148 - DNA, 52 bp, Homo sapiens
ctgaggtggtctcttagattcctggcycctaatgtacacaccccttcttcca

SEQ ID NO: 149 - DNA, 52 bp, Homo sapiens
gagaaagtcctataacaaattgatgamcttaagagcaaagtctgaggtcccc

SEQ ID NO: 150 - DNA, 52 bp, Homo sapiens
aacattttgttatgcttaaatgtctcraaaatgaattagaggccctaaaggg

SEQ ID NO: 151 - DNA, 52 bp, Homo sapiens
ttgctgtatatcagtctttcgatttcyttttgagaatgggagccttagtgca

SEQ ID NO: 152 - DNA, 52 bp, Homo sapiens
cttttttcctcctcagcaagtgactaycctgaaagcaatcatgttttcttgt

SEQ ID NO: 153 - DNA, 52 bp, Homo sapiens
tattttccctttgaagctcaccccagyacgtattgacaaggacaattgtagg

SEQ ID NO: 154 - DNA, 52 bp, Homo sapiens
caagggacattgcaaaagctagcttakggacttccccattcacagggagaag

SEQ ID NO: 155 - DNA, 52 bp, Homo sapiens
agttcattactcccatttcattcatcrgcaaataccgtattgtgatgataat

SEQ ID NO: 156 - DNA, 52 bp, Homo sapiens
gatatacgcagctgttaaaatcatgcrtacaggactattggttgaatagtcc

SEQ ID NO: 157 - DNA, 52 bp, Homo sapiens
cattgcaagacttccaggagtgcatcygtttctaatgtacagtgcataattt

SEQ ID NO: 158 - DNA, 52 bp, Homo sapiens
tttctccacttttcattctagttacamctaactactcattgttccctgaaaa

SEQ ID NO: 159 - DNA, 52 bp, Homo sapiens
gctggaaagacatgcttttaaaaaatkgtgctaaatatgtataacatacgat

SEQ ID NO: 160 - DNA, 52 bp, Homo sapiens
cctcctaagtcacattccttgtcactkactatcaaacattcaaaatgtatcc

SEQ ID NO: 161 - DNA, 52 bp, Homo sapiens
gaatgaatgaattctaagtcaatccargagtctgatgatttcttgaaaaggg

SEQ ID NO: 162 - DNA, 52 bp, Homo sapiens
ttatcacattttctcagacaatgtaayaggggatgctgcttgtcctcaacat

SEQ ID NO: 163 - DNA, 52 bp, Homo sapiens
gattcctgcttttattattatgaattytcagagtaatttctcccgcctcctg

SEQ ID NO: 164 - DNA, 52 bp, Homo sapiens
ctctgtggcttccttagatgttagaaytgggttatgcagaagtcattcagtt

SEQ ID NO: 165 - DNA, 52 bp, Homo sapiens
actgagagtatggagtatgtctccgaratacataggtgatgtgtattctaga

SEQ ID NO: 166 - DNA, 52 bp, Homo sapiens
aaatcctgcctctactctatcacttcrggcaggcaggtccttaggctctttg

SEQ ID NO: 167 - DNA, 52 bp, Homo sapiens
gtcgcaaaacaaaacaaaacaaaaccygctcaaatcgtgttaaaacaagcaa